(19) United States
(12) Patent Application Publication
(10) Pub. No.: US 2022/0056134 A1
(43) Pub. Date: Feb. 24, 2022

(54) METHODS OF MAKING ANTIBODIES

(71) Applicant: Genentech, Inc., South San Francisco, CA (US)

(72) Inventors: Kamal Kishore JOSHI, South San Francisco, CA (US); Paul J. Carter, Hillsborough, CA (US); Yiyuan Yin, Fremont, CA (US)

(73) Assignee: Genentech, Inc., South San Francisco, CA (US)

(21) Appl. No.: 17/454,015

(22) Filed: Nov. 8, 2021

Related U.S. Application Data
(63) Continuation of application No. PCT/US2020/031914, filed on May 7, 2020.
(60) Provisional application No. 62/845,594, filed on May 9, 2019.

(51) Int. Cl. C07K 16/28; C12N 5/10

(52) U.S. Cl. CPC C07K 16/2809; C07K 2317/31; C07K 16/2833

(57) ABSTRACT

Provided are, inter alia, methods of improving pairing of a heavy chain and a light chain of an antibody (such as a bispecific antibody). Also provided are antibodies (e.g., bispecific antibodies) generated using such methods, libraries, and methods of screening such libraries.

### CROSS REFERENCE TO RELATED APPLICATIONS

This application claims the priority benefit of U.S. Provisional Application No. 62/845,594, filed on May 9, 2019, the contents of which are incorporated herein by reference in their entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 146392047740SEQLIST.TXT, date recorded: May 4, 2020, size: 9 KB).

### BACKGROUND

The development of bispecific antibodies as therapeutic agents for human diseases has great clinical potential. However, production of bispecific antibodies in IgG format has been challenging, as antibody heavy chains have evolved to bind antibody light chains in a relatively promiscuous manner. As a result of this promiscuous pairing, concomitant expression of two antibody heavy chains and two antibody light chains in a single cell naturally leads to, e.g., heavy chain homodimerization and scrambling of heavy chain/light chain pairings.

One approach to circumvent the problem of heavy chain homodimerization, known as ‘knobs-into-holes, aims at forcing the pairing of two different antibody heavy chains by introducing mutations into the C_{H}3 domains to modify the contact interface. On one heavy chain original amino acids were replaced by amino acids with short side chains to create a ‘hole’. Conversely, amino acids with large side chains were introduced into the other C_{H}3 domain, to create a ‘knob’. By coexpressing these two heavy chains (and two identical light chains, which have to be appropriate for both heavy chains), high yields of heterodimer formation (‘knob-hole’) versus homodimer formation (‘hole-hole’ or ‘knob-knob’) was observed (Ridgway, J. B., Protein Eng. 9 (1996) 617-621; Merchant et al. “An efficient route to human bispecific IgG.” Nat Biotechnol. 1998; 16:677-81; Jackman et al. “Development of a two-part strategy to identify a therapeutic human bispecific antibody that inhibits IgE receptor signaling.” J Biol Chem. 2010;285:20850-9; and WO 96/027011).

Minimizing the scrambling of heavy chain/light chain has been more difficult due to the complex multidomain heterodimeric interactions within antibody Fabs. Bispecific antibodies formats aimed at addressing heavy chain/light scrambling include: DVD-Ig (Dual Variable Domain Ig) (Nature Biotechnology 25, 1290-1297 (2007)); Cross-over Ig (CROSSMAB™) (Schaefer W et al (2011) PNAS 108(27): 11187-11192); Two-in-One Ig (Science 2009, 323, 1610); BiTE® antibodies (PNAS 92(15):7021-7025; 1995) and strategies described in Lewis et al. (2014) “Generation of bispecific IgG antibodies by structure-based design of an orthogonal Fab interface.” Nat Biotechnol 32, 191-8; Liu et al. (2015) “A Novel Antibody Engineering Strategy for Making Monovalent Bispecific Heterodimeric IgG Antibodies by Electrostatic Steering Mechanism.” J Biol Chem. Published online Jan. 12, 2015, doi:10.1074/jbc.M114.620260; Mazor et al. 2015. “Improving target cell specificity using a novel monovalent bispecific IgG design.” Mabs. Published online January 26, 2015, doi: 10.1080/19420862.2015.1007816; WO 2014/081955, WO 2014/082179, and WO 2014/150973.

There nevertheless remains a need in the art for methods of reducing mispaired heavy chain/light chain by-products and increase yield of correctly assembled bispecific antibody.

### BRIEF SUMMARY OF THE INVENTION

Provided is a method of improving preferential pairing of a heavy chain and a light chain of an antibody, comprising the step of substituting at least one amino acid at position 94 of a light chain variable domain (V_{L}) or position 96 of the V_{L}, from a non-charged residue to a charged residue selected from the group consisting of aspartic acid (D), arginine (R), glutamic acid (E), and lysine (K), wherein the amino acid numbering is according to Kabat. In some embodiments, the method comprises the step of substituting each of the amino acids at position 94 and position 96 from a non-charged residue to a charged residue. In some embodiments, the amino acid at position 94 is substituted with D. In some embodiments, the amino acid at position 96 is substituted with R. In some embodiments, the amino acid at position 94 is substituted with D and the amino acid at position 96 is substituted with R. In some embodiments, the amino acid at position 95 of a heavy chain variable domain (V_{H}) is substituted from a non-charged residue to a charged residue selected from the group consisting of aspartic acid (D), arginine (R), glutamic acid (E), and lysine (K), wherein the amino acid numbering is according to Kabat. In some embodiments, the amino acid at position 94 of the V_{L} is substituted with D, the amino acid at position 96 of the V_{L} is substituted with R, and the amino acid at position 95 of the V_{H} is substituted with D.

In some embodiments, a method provided herein further comprises subjecting the antibody (e.g., the antibody that has been modified to improve preferential pairing of the heavy chain and the light chain) to at least one affinity maturation step, wherein the substituted amino acid at position 94 of the V_{L} is not randomized. Additionally or alternatively, in some embodiments, the substituted amino acid at position 96 of the V_{L} is not randomized. Additionally or alternatively, in some embodiments, the substituted amino acid at position 95 of the V_{H} is not randomized.

In some embodiments, the antibody is an antibody fragment selected from the group consisting of: a Fab, a Fab′, an F(ab′)₂, a one-armed antibody, and scFv, or an Fv. In some embodiments, the antibody is a human, humanized, or chimeric antibody. In some embodiments, the antibody comprises a human IgG Fc region. In some embodiments, the human IgG Fc region is a human IgG1, human IgG2, human IgG3, or human IgG4 Fc region. In some embodiments, the antibody is a monospecific antibody. In some embodiments, the antibody is a multispecific antibody.

In some embodiments, the multispecific antibody is a bispecific antibody. In some embodiments, the bispecific antibody comprises a first C_{H}2 domain (C_{H}2₁), a first C_{H}3 domain (C_{H}3₁), a second C_{H}2 domain (C_{H}2₂), and a second C_{H}3 domain; wherein C_{H}3₂ is altered so that within the C_{H}3₁/C_{H}3₂ interface, one or more amino acid residues are replaced with one or more amino acid residues having a larger side chain volume, thereby generating a protuberance on the surface of C_{H}3₂ that interacts with C_{H}3₁; and wherein C_{H}3₁ is altered so that within the C_{H}3₁/C_{H}3₂ interface, one or more amino acid residues are replaced amino acid residues having a smaller side chain volume, thereby generating a cavity on the surface of C_{H}3₁ that interacts with C_{H}3₂. In some embodiments, the bispecific antibody comprises a first C_{H}2 domain (C_{H}2₁), a first C_{H}3 domain (C_{H}3₁), a second C_{H}2 domain (C_{H}2₂), and a second C_{H}3 domain; wherein C_{H}3₁ is altered so that within the C_{H}3₁/C_{H}3₂ interface, one or more amino acid residues are replaced with one or more amino acid residues having a larger side chain volume, thereby generating a protuberance on the surface of C_{H}3₁ that interacts with C_{H}3₂; and wherein C_{H}3₂ is altered so that within the C_{H}3₁/C_{H}3₂ interface, one or more amino acid residues are replaced amino acid residues having a smaller side chain volume, thereby generating a cavity on the surface of C_{H}3₂ that interacts with C_{H}3₁. In some embodiments, the protuberance is a knob mutation. In some embodiments, the knob mutation comprises T366W, wherein amino acid numbering is according to the EU index. In some embodiments, the cavity is a hole mutation. In some embodiments, the hole mutation comprises at least one, at least two, or all three of T366S, L368A, and Y407V, wherein amino acid numbering is according to the EU index.

Also provided is an antibody produced by any one (or combination) of the methods described herein.

### BRIEF DESCRIPTION OF THE DRAWINGS

FIGS. 1A and 1B provide high resolution liquid chromatography mass spectrometry (LCMS) data for an anti-LGR5/anti-IL4 bispecific antibody, i.e., a representative example of a low-yield BsIgG. FIG. 1A shows the mass envelopes for charge states 38+ and 39+. FIG. 1B shows corresponding deconvoluted data.

FIGS. 1C and 1D provide high resolution LCMS data for an anti-SIRPα/anti-IL4 bispecific antibody, i.e., a representative example of an intermediate yield BsIgG. FIG. 1C shows the mass envelopes for charge states 38+ and 39+. FIG. 1D shows corresponding deconvoluted data.

FIGS. 1E and 1F provide high resolution LCMS data for an anti-Met/anti-DR5 bispecific antibody, i.e., a representative example of a high yield BsIgG. FIG. 1E shows the mass envelopes for charge states 38+ and 39+. FIG. 1F shows corresponding deconvoluted data.

FIG. 2 provides the results of experiments that were performed to determine whether incorporating C_{H}1/C_{L} charge pair substitution mutations increases yield for BsIgG that demonstrate a strong intrinsic HC/LC pairing preference.

FIG. 3 illustrates the design of experiments that were performed to investigate the mechanistic basis for preferential HC/LC pairing in an anti-EGFR/anti-MET BsIgG and an anti-IL-4/anti-IL-13 BsIgG. The results of this experiment are provided in Table C.

FIG. 4A provides an alignment of the light chain variable domains (V_{L}) of the anti-MET antibody onartuzumab (see Merchant et al. (2013) PNAS USA 110: E2987-2996) (SEQ ID NO: 1) and the anti-EGFR antibody D1.5 (see Schaefer et al. (2011) Cancer Cell 20: 472-486) (SEQ ID NO: 2). Amino acid residues are numbered according to Kabat. CDRs from the sequence definition of Kabat et al. Sequences of Proteins of Immunological Interest. Bethesda, Md.: NIH, 1991 and the structural definition of Chothia and Lesk (1987) J Mol Biol 196: 901-917 are shaded.

FIG. 4B provides an alignment of the heavy chain variable domains (V_{H}) of the anti-MET antibody onartuzumab (SEQ ID NO: 3) and the anti-EGFR antibody D1.5 (SEQ ID NO: 4). Amino acid residues are numbered according to Kabat. CDRs from the sequence definition of Kabat et al. Sequences of Proteins of Immunological Interest. Bethesda, Md.: NIH, 1991 and the structural definition of Chothia and Lesk (1987) J Mol Biol 196: 901-917 are shaded.

FIG. 5A provides the results of experiments that were performed to assess the contributions of complementarity determining region (CDR) L3 and CDR H3 of the anti-EGFR arm of an anti-EGFR/anti-MET bispecific antibody to BsIgG yield. Also provided are the results of experiments performed to assess the contributions of CDR L3 and CDR H3 of the anti-MET arm of an anti-EGFR/anti-MET bispecific antibody to BsIgG yield.

FIG. 5B provides the results of experiments that were performed to assess the contributions of CDR L3 and CDR H3 of the anti-IL-4 arm of an anti-IL-4/anti-IL-13 bispecific antibody to BsIgG yield. Also provided are the results of experiments that were performed to assess the contributions of CDR L3 and CDR H3 of the anti-IL-13 arm of an anti-IL-4/anti-IL-13 bispecific antibody to BsIgG yield.

FIG. 6 provides the results of experiments that were performed to assess the contributions of CDR-L1 + CDR-H1, CDR-L2 + CDR-H2, and CDR-L3 + CDR-H3 on BsIgG yield of the anti-EGFR/anti-MET bispecific antibody.

FIG. 7 provides an X-ray structure of the anti-MET Fab (PDB 4K3J) highlighting CDR L3 and CDR H3 contact residues.

FIG. 8A provides an alignment of the light chain variable domains (V_{L}) of the anti-IL-13 antibody lebrikizumab (see Ultsch et al. (2013) J Mol Biol 425: 1330-1339) (SEQ ID NO: 5) and the anti-IL-4 antibody 19C11 (see Spiess et al. (2013) J Biol Chem 288: 265:83-93) (SEQ ID NO: 6). CDRs from the sequence definition of Kabat and the structural definition of Chothia and Lesk are shaded.

FIG. 8B provides an alignment of the heavy chain variable domains (V_{H}) of the anti-IL-13 antibody lebrikizumab (SEQ ID NO: 7) and the anti-IL-4 antibody 19C11 (SEQ ID NO: 8). Amino acid residues are numbered according to Kabat. CDRs from the sequence definition of Kabat and the structural definition of Chothia and Lesk are shaded.

FIG. 9 provides an X-ray structure of the anti-IL-13 Fab (PDB 4177) highlighting CDR L3 and CDR H3 contact residues.

FIG. 10A provides the results of experiments that were performed to assess the effect of (a) replacing the CDR L3 and CDR H3 of the anti-CD3 arm of an anti-CD3/anti-HER2 bispecific antibody with the CDR L3 and CDR H3 of anti-MET; (b) replacing the CDR L3 and CDR H3 of the anti-HER2 arm of an anti-CD3/anti-HER2 bispecific antibody with the CDR L3 and CDR H3 of anti-MET; (c) replacing the CDR L3 and CDR H3 of the anti-CD3 arm of an anti-CD3/anti-HER2 bispecific antibody with the CDR L3 and CDR H3 of anti-IL-13; and (d) replacing the CDR L3 and CDR H3 of the anti-HER2 arm of an anti-CD3/anti-HER2 bispecific antibody with the CDR L3 and CDR H3 of anti-IL-13 on BsIgG yield.

FIG. 10B provides the results of experiments that were performed to assess the effect of (a) replacing the CDR L3 and CDR H3 of the anti-VEGFA arm of an anti-VEGFA/anti-ANG2 bispecific antibody with the CDR L3 and CDR H3 of anti-MET; (b) replacing the CDR L3 and CDR H3 of the anti-ANG2 arm of an anti-VEGFA/anti-ANG2 bispecific antibody with the CDR L3 and CDR H3 of anti-MET; (c) replacing the CDR L3 and CDR H3 of the anti-VEGFA arm of an anti-VEGFA/anti-ANG2 bispecific antibody with the CDR L3 and CDR H3 of anti-IL-13; and (d) replacing the CDR L3 and CDR H3 of the anti-ANG2 arm of an anti-VEGFA/anti-ANG2 bispecific antibody with the CDR L3 and CDR H3 of anti-IL-13 on BsIgG yield.

FIG. 11 provides the results of experiments that were performed to assess the contribution of interchain disulfide bonds on BsIgG yield of the following bispecific antibodies: (1) anti-HER2/anti-CD3; (2) anti-VEGFA/anti-VEGFC; (3) anti-EGFR/anti-MET; and (4) anti-IL13/anti-IL-4.

### DETAILED DESCRIPTION OF THE INVENTION

Bispecific antibodies are promising class of therapeutic agents, as their dual specificity permits, e.g., delivering payloads to targeted sites, simultaneous blocking of two signaling pathways, delivering immune cells to tumor cells, etc. However, the production of bispecific antibodies (e.g., bispecific IgGs, or “BsIgGs”) remains a technical challenge, as co-expression of two antibody heavy chains and two antibody light chains in a single cell may naturally lead to, e.g., heavy chain homodimerization and scrambling of heavy chain/light chain pairings. The methods described herein are based on Applicant's finding that preferential antibody heavy chain/antibody light chain can be strongly influenced by residues at specific amino acid positions in the CDR-H3 and CDR-L3. Moreover, Applicant found that transfer of such residues to corresponding amino acid positions in other unrelated antibodies increased yields of correctly assembled BsIgG in many cases.

Unless defined otherwise herein, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D Ed., John Wiley and Sons, New York (1994), and Hale & Margham, THE HARPER COLLINS DICTIONARY OF BIOLOGY, Harper Perennial, NY (1991) provide one of skill with a general dictionary of many of the terms used in this invention. Although any methods and materials similar or equivalent to those described herein can be used in the practice or testing of the present invention, the preferred methods and materials are described. Numeric ranges are inclusive of the numbers defining the range. Unless otherwise indicated, nucleic acids are written left to right in 5′ to 3′ orientation; amino acid sequences are written left to right in amino to carboxy orientation, respectively. Practitioners are particularly directed to Sambrook et al., 1989, and Ausubel FM et al., 1993, for definitions and terms of the art. It is to be understood that this invention is not limited to the particular methodology, protocols, and reagents described, as these may vary.

Numeric ranges are inclusive of the numbers defining the range.

Unless otherwise indicated, nucleic acids are written left to right in 5′ to 3′ orientation; amino acid sequences are written left to right in amino to carboxy orientation, respectively.

The headings provided herein are not limitations of the various aspects or embodiments which can be had by reference to the specification as a whole. Accordingly, the terms defined immediately below are more fully defined by reference to the specification as a whole.

### Definitions

The term “antibody” herein is used in the broadest sense and refers to any immunoglobulin (Ig) molecule comprising two heavy chains and two light chains, and any fragment, mutant, variant or derivation thereof so long as they exhibit the desired biological activity (e.g., epitope binding activity). Examples of antibodies include monoclonal antibodies, polyclonal antibodies, multispecific antibodies (e.g., bispecific antibodies) and antibody fragments as described herein. An antibody can be mouse, chimeric, human, humanized and/or affinity matured.

As a frame of reference, as used herein an immunoglobulin will refer to the structure of an immunoglobulin G (IgG). However, one skilled in the art would understand/recognize that an antibody of any immunoglobulin class may be utilized in the inventive method described herein. For clarity, an IgG molecule contains a pair of heavy chains (HCs) and a pair of light chains (LCs). Each LC has one variable domain (V_{L}) and one constant domain (CL), while each HC has one variable (V_{H}) and three constant domains (C_{H}1, C_{H}2, and C_{H}3). The C_{H}1 and C_{H}2 domains are connected by a hinge region. This structure is well known in the art.

Briefly, the basic 4-chain antibody unit is a heterotetrameric glycoprotein composed of two light (L) chains and two heavy (H) chains (an IgM antibody consists of 5 of the basic heterotetramer unit along with an additional polypeptide called J chain, and therefore contain 10 antigen binding sites, while secreted IgA antibodies can polymerize to form polyvalent assemblages comprising 2-5 of the basic 4-chain units along with J chain). In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (V_{H}) followed by three constant domains (C_{H}) for each of the α and γ chains and four C_{H} domains for μ and ϵ isotypes. Each L chain has at the N-terminus, a variable domain (V_{L}) followed by a constant domain (C_{L}) at its other end. The V_{L} is aligned with the V_{H} and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71 and Chapter 6.

The L chain from any vertebrate species can be assigned to one of two clearly distinct types, called kappa and lambda, based on the amino acid sequences of their constant domains. Depending on the amino acid sequence of the constant domain of their heavy chains (C_{H}), immunoglobulins can be assigned to different classes or isotypes. There are five classes of immunoglobulins: IgA, IgD, IgE, IgG, and IgM, having heavy chains designated α, δ, γ, ϵ, and μ, respectively. The γ and α classes are further divided into subclasses on the basis of relatively minor differences in C_{H} sequence and function, e.g., humans express the following subclasses: IgG1, IgG2, IgG3, IgG4, IgA1, and IgA2.

The term “CL domain” comprises the constant region domain of an immunoglobulin light chain that extends, e.g. from about Kabat position 107A-216 (EU positions 108-214 (kappa)). The Eu/Kabat conversion table for the Kappa C domain is available online at www(dot)imgt(dot)org/IMGTScientificChart/Numbering/Hu_IGKCnber.html, and the Eu/Kabat conversion table for the Lambda C domain is available online at www(dot)imgt(dot)org/IMGTScientificChart/Numbering/Hu_IGLCnber.html. The C_{L} domain is adjacent to the V_{L} domain and includes the carboxy terminal of an immunoglobulin light chain.

As used herein, the term “C_{H}1 domain” of a human IgG comprises the first (most amino terminal) constant region domain of an immunoglobulin heavy chain that extends, e.g., from about positions 114-223 in the Kabat numbering system (EU positions 118-215). The C_{H}1 domain is adjacent to the V_{H} domain and amino terminal to the hinge region of an immunoglobulin heavy chain molecule, does not form a part of the Fc region of an immunoglobulin heavy chain, and is capable of dimerizing with an immunoglobulin light chain constant domain (i.e., “CL”). The EU/Kabat conversion tables for the IgG1 heavy chain is available online at www(dot)imgt(dot)org/IMGTScientificChart/Numbering/Hu_IGHGnber.html.

The term “C_{H}2 domain” of a human IgG Fc region usually comprises about residues 231 to about 340 of the IgG according to the EU numbering system. The C_{H}2 domain is unique in that it is not closely paired with another domain. Rather, two N-linked branched carbohydrate chains are interposed between the two C_{H}2 domains of an intact native IgG molecule. It has been speculated that the carbohydrate may provide a substitute for the domain-domain pairing and help stabilize the C_{H}2 domain. Burton, Mol. Immunol. 22:161-206 (1985).

The term “C_{H}3 domain” comprises residues C-terminal to a C_{H}2 domain in an Fc region (i.e., from about amino acid residue 341 to about amino acid residue 447 of an IgG according to the EU numbering system).

The term “Fc region,” as used herein, generally refers to a dimer complex comprising the C-terminal polypeptide sequences of an immunoglobulin heavy chain, wherein a C-terminal polypeptide sequence is that which is obtainable by papain digestion of an intact antibody. The Fc region may comprise native or variant Fc sequences. Although the boundaries of the Fc sequence of an immunoglobulin heavy chain might vary, the human IgG heavy chain Fc sequence comprises about position Cys226, or from about position Pro230, to the carboxyl terminus of the Fc sequence. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991. The Fc sequence of an immunoglobulin generally comprises two constant domains, a C_{H}2 domain and a C_{H}3 domain, and optionally comprises a C_{H}4 domain. By “Fc polypeptide” herein is meant one of the polypeptides that make up an Fc region, e.g., a monomeric Fc. An Fc polypeptide may be obtained from any suitable immunoglobulin, such as human IgG1, IgG2, IgG3, or IgG4 subtypes, IgA, IgE, IgD or IgM. An Fc polypeptide may be obtained from mouse, e.g., a mouse IgG2a. The Fc region comprises the carboxy-terminal portions of both H chains held together by disulfides. The effector functions of antibodies are determined by sequences in the Fc region; this region is also the part recognized by Fc receptors (FcR) found on certain types of cells. In some embodiments, an Fc polypeptide comprises part or all of a wild type hinge sequence (generally at its N terminus). In some embodiments, an Fc polypeptide does not comprise a functional or wild type hinge sequence.

“Fc component” as used herein refers to a hinge region, a C_{H}2 domain or a C_{H}3 domain of an Fc region.

In certain embodiments, the Fc region comprises an IgG Fc region, preferably derived from a wild-type human IgG Fc region. In certain embodiments, the Fc region is derived from a “wild type” mouse IgG, such as a mouse IgG2a. By “wild-type” human IgG Fc or “wild type” mouse IgG Fc it is meant a sequence of amino acids that occurs naturally within the human population or mouse population, respectively. Of course, just as the Fc sequence may vary slightly between individuals, one or more alterations may be made to a wild type sequence and still remain within the scope of the invention. For example, the Fc region may contain alterations such as a mutation in a glycosylation site or inclusion of an unnatural amino acid.

The term “variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three hypervariable regions (HVRs). (See, e.g., Kindt et al. Kuby, Immunology, 61st ed., W.H. Freeman and Co., page 91 (2007).) A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J. Immunol. 150:880-887 (1993); Clarkson et al., Nature 352:624-628 (1991).

The term “hypervariable region” or “HVR” as used herein refers to each of the regions of an antibody variable domain which are hypervariable in sequence and which determine antigen binding specificity, for example “complementarity determining regions” (“CDRs”).

Generally, antibodies comprise six CDRs: three in the V_{H} (CDR-H1, CDR-H2, CDR-H3), and three in the V_{L} (CDR-L1, CDR-L2, CDR-L3). Exemplary CDRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, Md. (1991)); and
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)).

Unless otherwise indicated, the CDRs are determined according to Kabat et al., supra. One of skill in the art will understand that the CDR designations can also be determined according to Chothia, supra, McCallum, supra, or any other scientifically accepted nomenclature system.

“Framework” or “FR” refers to variable domain residues other than complementary determining regions (CDRs). The FR of a variable domain generally consists of four FR domains: FR1, FR2, FR3, and FR4. Accordingly, the CDR and FR sequences generally appear in the following sequence in V_{H} (or V_{L}): FR1-CDR-H1(CDR-L1)-FR2-CDR-H2(CDR-L2)-FR3- CDR-H3(CDR-L3)-FR4.

The phrase “antigen binding arm,” “target molecule binding arm,” “target binding arm” and variations thereof, as used herein, refers to a component part of an antibody (such as a bispecific antibody) that has an ability to specifically bind a target of interest. Generally and preferably, the antigen binding arm is a complex of immunoglobulin polypeptide sequences, e.g., CDR and/or variable domain sequences of an immunoglobulin light and heavy chain.

A “target” or “target molecule” refers to a moiety recognized by a binding arm of an antibody (such as a bispecific antibody). For example, if the antibody is a multispecific antibody (e.g., a bispecific antibody), then the target may be epitopes on a single molecule or on different molecules, or a pathogen or a tumor cell, depending on the context. One skilled in the art will appreciate that the target is determined by the binding specificity of the target binding arm and that different target binding arms may recognize different targets. A target preferably binds to an antibody (e.g., a bispecific antibody) with affinity higher than 1 μM Kd (according to methods known in the art, including the methods described herein). Examples of target molecules include, but are not limited to, serum soluble proteins and/or their receptors, such as cytokines and/or cytokine receptors, adhesins, growth factors and/or their receptors, hormones, viral particles (e.g., RSV F protein, CMV, Staph A, influenza, hepatitis C virus), micoorganisms (e.g., bacterial cell proteins, fungal cells), adhesins, CD proteins and their receptors.

The term “interface” as used herein refers to the association surface that results from interaction of one or more amino acids in a first antibody domain with one or more amino acids of a second antibody domain. Exemplary interfaces include, e.g., C_{H}1/C_{L}, V_{H}/V_{L} and C_{H}3/C_{H}3. In some embodiments, the interface includes, for example, hydrogen bonds, electrostatic interactions, or salt bridges between the amino acids forming an interface.

One example of an “intact” or “full-length” antibody is one that comprises an antigen-binding arm as well as a C_{L} and at least heavy chain constant domains, C_{H}1, C_{H}2, and C_{H}3. The constant domains can be native sequence constant domains (e.g., human native sequence constant domains) or amino acid sequence variants thereof.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical composition.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable domain (V_{H}), also called a variable heavy domain or a heavy chain variable region, followed by three constant heavy domains (C_{H}1, C_{H}2, and C_{H}3). Similarly, from N- to C-terminus, each light chain has a variable domain (V_{L}), also called a variable light domain or a light chain variable region, followed by a constant light (C_{L}) domain.

“Monospecific” refers to the ability of an antibody, to bind only one epitope. “Bispecific” refers to the ability of an antibody to bind two different epitopes. “Multispecific” refers to the ability of an antibody to bind more than one epitope. In certain embodiments, a multispecific antibody encompasses a bispecific antibody. For bispecific and multispecific antibodies, the epitopes can be on the same antigen, or each epitope can be on a different antigen. In certain embodiments, a bispecific antibody binds to two different antigens. In certain embodiments, a bispecific antibody, binds to two different epitopes on one antigen. In certain embodiments, a multispecific antibody (such as a bispecific antibody) binds to each epitope with a dissociation constant (Kd) of about ≤1μM, about ≤100 nM, about ≤10 nM, about ≤1 nM, about ≤0.1 nM, about ≤0.01 nM, or about ≤0.001 nM (e.g., about 10⁻⁸M or less, e.g., from about 10⁻⁸M to about 10⁻¹³M, e.g., from about 10⁻⁹M to about 10⁻¹³ M).

The term “multispecific antibody” herein is used in the broadest sense refers to an antibody capable of binding two or more antigens. In certain aspects the multispecific antibody refers to a bispecific antibody, e.g., a human bispecific antibody, a humanized bispecific antibody, a chimeric bispecific antibody, or a mouse bispecific antibody.

“Antibody fragments” comprise a portion of an intact antibody, preferably the V_{H} and V_{L} of the intact antibody. Examples of antibody fragments include Fab, Fab′, F(ab′)2, ScFv, and Fv fragments; one-armed antibodies, and multispecific antibodies formed from antibody fragments.

Antibodies can be “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, provided that they exhibit the desired biological activity (U.S. Patent No. 4,816,567; and Morrison et al., Proc. Natl. Acad. Sci. USA 81 :6851-6855 (1984)).Chimeric antibodies of interest herein include primatized antibodies comprising variable domain antigen-binding sequences derived from a non-human primate (e.g., Old World Monkey, Ape, etc.) and human constant region sequences.

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies can comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a nonhuman immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

The term “pharmaceutical composition” or “pharmaceutical formulation” refers to a preparation which is in such form as to permit the biological activity of an active ingredient contained therein to be effective, and which contains no additional components which are unacceptably toxic to a subject to which the pharmaceutical composition would be administered.

A “pharmaceutically acceptable carrier” refers to an ingredient in a pharmaceutical composition or formulation, other than an active ingredient, which is nontoxic to a subject. A pharmaceutically acceptable carrier includes, but is not limited to, a buffer, excipient, stabilizer, or preservative.

“Complex” or “complexed” as used herein refers to the association of two or more molecules that interact with each other through bonds and/or forces (e.g., van der Waals, hydrophobic, hydrophilic forces) that are not peptide bonds. In one embodiment, the complex is heteromultimeric. It should be understood that the term “protein complex” or “polypeptide complex” as used herein includes complexes that have a non-protein entity conjugated to a protein in the protein complex (e.g., including, but not limited to, chemical molecules such as a toxin or a detection agent).

An antibody (such as a monospecific or multispecific antibody) “which binds an antigen of interest” is one that binds the antigen, e.g., a protein, with sufficient affinity such that the antibody is useful as a diagnostic and/or therapeutic agent in targeting a protein or a cell or tissue expressing the protein, and does not significantly cross-react with other proteins. In such embodiments, the extent of binding of the antibody to a “non-target” protein will be less than about 10% of the binding of the antibody to its particular target protein as determined by fluorescence activated cell sorting (FACS) analysis or radioimmunoprecipitation (RIA) or ELISA. With regard to the binding of antibody to a target molecule, the term “specific binding” or “specifically binds to” or is “specific for” a particular polypeptide or an epitope on a particular polypeptide target means binding that is measurably different from a nonspecific interaction (e.g., a non-specific interaction may be binding to bovine serum albumin or casein). Specific binding can be measured, for example, by determining binding of a molecule compared to binding of a control molecule. For example, specific binding can be determined by competition with a control molecule that is similar to the target, for example, an excess of non-labeled target. In this case, specific binding is indicated if the binding of the labeled target to a probe is competitively inhibited by excess unlabeled target. The term “specific binding” or “specifically binds to” or is “specific for” a particular polypeptide or an epitope on a particular polypeptide target as used herein can be exhibited, for example, by a molecule having a Kd for the target of at least about 200 nM, alternatively at least about 150 nM, alternatively at least about 100 nM, alternatively at least about 60 nM, alternatively at least about 50 nM, alternatively at least about 40 nM, alternatively at least about 30 nM, alternatively at least about 20 nM, alternatively at least about 10 nM, alternatively at least about 8 nM, alternatively at least about 6 nM, alternatively at least about 4 nM, alternatively at least about 2 nM, alternatively at least about 1 nM, or greater affinity. In one embodiment, the term “specific binding” refers to binding where a multispecific antibody binds to a particular polypeptide or epitope on a particular polypeptide without substantially binding to any other polypeptide or polypeptide epitope.

“Binding affinity” generally refers to the strength of the sum total of noncovalent interactions between a single binding site of a molecule (e.g., an antibody such as a bispecific or multispecific antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). For example, the Kd can be about 200 nM or less, about 150 nM or less, about 100 nM or less, about 60 nM or less, about 50 nM or less, about 40 nM or less, about 30 nM or less, about 20 nM or less, about 10 nM or less, about 8 nM or less, about 6 nM or less, about 4 nM or less, about 2 nM or less, or about 1 nM or less. Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention.

In one embodiment, the “Kd” or “Kd value” is measured by using surface plasmon resonance assays. For example, the Kd value can be determined using a BIAcore™-2000 or a BIAcore™-3000 (BIAcore, Inc., Piscataway, N.J.) at 25° C. with immobilized target (e.g., antigen) CM5 chips at -10 response units (RU). Briefly, in one example, carboxymethylated dextran biosensor chips (CM5, BIAcore Inc.) are activated with N-ethyl-N′- (3- dimethylaminopropyl)-carbodiimide hydrochloride (EDC) and N-hydroxysuccinimide (NHS) according to the supplier's instructions. Antigen is diluted with 10 mM sodium acetate, pH 4.8, into 5 μl g/ml (˜0.2 μM) before injection at a flow rate of 5 μl/minute to achieve approximately 10 response units (RU) of coupled protein. Following the injection of antigen, 1M ethanolamine is injected to block unreacted groups. For kinetics measurements, two-fold serial dilutions of Fab (e.g., 0.78 nM to 500 nM) are injected in PBS with 0.05% Tween 20 (PBST) at 25° C. at a flow rate of approximately 25 μl/min. Association rates (kₒₙ) and dissociation rates (k_{off}) are calculated using a simple one-to-one Langmuir binding model (BIAcore Evaluation Software version 3.2) by simultaneous fitting the association and dissociation sensorgram. The equilibrium dissociation constant (Kd) is calculated as the ratio k_{off}/kₒₙ. See, e.g., Chen et al., J. Mol. Biol. 293:865-881 (1999). If the on-rate exceeds 10⁶M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophotometer (Aviv Instruments) or a 8000-series SLM-Aminco spectrophotometer (ThermoSpectronic) with a stirred cuvette.

“Biologically active” and “biological activity” and “biological characteristics” with respect to an antibody (e.g., a modified antibody, such as a modified bispecific antibody) made according to a method provided herein, such as an antibody (e.g., a bispecific antibody), fragment, or derivative thereof, means having the ability to bind to a biological molecule, except where specified otherwise.

“Isolated,” when used to describe the various heteromultimer polypeptides means a heteromultimer which has been separated and/or recovered from a cell or cell culture from which it was expressed. Contaminant components of its natural environment are materials which would interfere with diagnostic or therapeutic uses for the heteromultimer, and may include enzymes, hormones, and other proteinaceous or nonproteinaceous solutes. In certain embodiments, the heteromultimer will be purified (1) to greater than 95% by weight of protein as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS PAGE under reducing or nonreducing conditions using Coomassie blue or, preferably, silver stain. Ordinarily, however, isolated polypeptide will be prepared by at least one purification step.

An antibody (such as a bispecific antibody) is generally purified to substantial homogeneity. The phrases “substantially homogeneous,” “substantially homogeneous form,” and “substantial homogeneity” are used to indicate that the product is substantially devoid of by-products originated from undesired polypeptide combinations (e.g., heavy chain homodimers and/or scrambled heavy chain/light chain pairs).

Expressed in terms of purity, substantial homogeneity means that the amount of by-products does not exceed 10%, 9%, 8%, 7%, 6%, 4%, 3%, 2% or 1% by weight or is less than 1% by weight. In one embodiment, the by-product is below 5%.

“Biological molecule” refers to a nucleic acid, a protein, a carbohydrate, a lipid, and combinations thereof. In one embodiment, the biologic molecule exists in nature.

Except where indicated otherwise by context, the terms “first” polypeptide (such as a heavy chain (HC1 or HC₁) or light chain (LC1 or LC₁)) and “second” polypeptide (such as a heavy chain (HC2 or HC₂) or light chain (LC2 or LC₂)), and variations thereof, are merely generic identifiers, and are not to be taken as identifying a specific or a particular polypeptide or component of an antibody (such as bispecific antibody) generated using a method provided herein.

Commercially available reagents referred to in the Examples were used according to manufacturer's instructions unless otherwise indicated. The source of those cells identified in the following Examples, and throughout the specification, by ATCC accession numbers is the American Type Culture Collection, Manassas, VA. Unless otherwise noted, the present invention uses standard procedures of recombinant DNA technology, such as those described hereinabove and in the following textbooks: Sambrook et al., supra; Ausubel et al., Current Protocols in Molecular Biology (Green Publishing Associates and Wiley Interscience, NY, 1989); Innis et al., PCR Protocols: A Guide to Methods and Applications (Academic Press, Inc., NY, 1990); Harlow et al., Antibodies: A Laboratory Manual (Cold Spring Harbor Press, Cold Spring Harbor, 1988); Gait, Oligonucleotide Synthesis (IRL Press, Oxford, 1984); Freshney, Animal Cell Culture, 1987; Coligan et al., Current Protocols in Immunology, 1991.

Reference to “about” a value or parameter herein refers to the usual error range for the respective value readily known to the skilled person in this technical field. Reference to “about” a value or parameter herein includes (and describes) aspects that are directed to that value or parameter per se. For example, description referring to “about X” includes description of “X.”

It is understood that aspects and embodiments of the invention described herein include “comprising,” “consisting of,” and “consisting essentially of” aspects and embodiments.

All references cited herein, including patent applications and publications, are hereby incorporated by reference in their entirety.

Methods of Improving Heavy Chain/Light Chain Pairing Selectivity

The present application is based on the identification of residues at amino acid positions in the V_{L} (e.g., of an antibody light chain or fragment thereof) and V_{H} (e.g., of an antibody heavy chain or fragment thereof) that play a role in preferential heavy chain/light chain pairing

As described in further detail below, the methods provided herein comprise introducing one or more substitutions at particular residues within the variable domains, e.g. in particular, within the CDR sequences, of heavy chain and/or light chain polypeptides. As one of ordinary skill in the art will appreciate, various numbering conventions may be employed for designating particular amino acid residues within antibody variable region sequences. Commonly used numbering conventions include Kabat and EU index numbering (see, Kabat et al., Sequences of Proteins of Immunological Interest, 5th Ed, Public Health Service, National Institutes of Health, Bethesda, Md. (1991)). Other conventions that include corrections or alternate numbering systems for variable domains include Chothia (Chothia C, Lesk A M (1987), J Mol Biol 196: 901-917; Chothia, et al. (1989), Nature 342: 877-883), IMGT (Lefranc, et al. (2003), Dev Comp Immunol 27: 55-77), and AHo (Honegger A, Plückthun A (2001) J Mol Biol 309: 657-670). These references provide amino acid sequence numbering schemes for immunoglobulin variable regions that define the location of variable region amino acid residues of antibody sequences.

Unless otherwise expressly stated herein, all references to immunoglobulin heavy chain variable region (i.e., V_{H}) amino acid residues (i.e. numbers) appearing in the Examples and Claims are based on the Kabat numbering system, as are all references to V_{L} residues, unless specifically indicated otherwise. All references to immunoglobulin heavy chain constant region C_{H}1, C_{H}2, and C_{H}3 residues (i.e., numbers) appearing in the Examples and Claims are based on the EU system, as are all references to C_{L} residues, unless specifically indicated otherwise. With knowledge of the residue number according to Kabat or EU Index numbering, one of ordinary skill can identify amino acid sequence modifications described herein, according to any commonly used numbering convention.

Although items, components, or elements provided herein (such as “antibody,” “substitution,” or “substitution mutation”) may be described or claimed in the singular, the plural is contemplated to be within the scope thereof unless limitation to the singular is explicitly stated.

As described in more detail below, provided herein are methods of improving correct heavy chain/light chain pairing in an antibody (including a bispecific antibody) that comprise introducing one or more substitutions into the V_{H} and/or V_{L}. Also provided are methods of improving yield of antibody (e.g., correctly assembled bispecific antibody) that comprise introducing one or more substitutions into the V_{H} and/or V_{L} of the antibody, wherein the yield of the antibody (e.g., bispecific antibody) comprising the substitutions produced using a particular method (e.g., a method known in the art) is higher than the yield of an unsubstituted antibody (e.g., bispecific antibody) produced using the same method. Previous efforts focused on introducing one or more amino acid substitutions into the framework regions of the variable domains. See, e.g., Froning et al., Protein Science, 2017, 26:2021-38. Liu et al., J. Biol. Chem. 2015, 290:7535-62. Lewis et al., Nature Biotechnology, 2014, 32:191-202.

In some embodiments, the methods provided herein further comprise introducing modification(s) in the Fc region to facilitate heterodimerization of the two heavy chains of an antibody (such as a bispecific antibody).

Substitution Mutations in the Heavy Chain and Light Chain Variable Domains

Provided herein is a method of improving the pairing (such as preferential pairing) of a heavy chain and a light chain of an antibody that comprises the step of substituting at least one amino acid (e.g., “original amino acid”) at position 94 of the light chain variable domain (V_{L}) or position 96 of the V_{L} from a non-charged residue to a charged residue selected from aspartic acid (D), arginine (R), glutamic acid (E), and lysine (K), wherein the amino acid numbering is according to Kabat. In some embodiments, the method comprises the step of substituting both the amino acids (e.g., original amino acids) at position 94 and position 96 from a non-charged residue to a charged residue, e.g., D, R, E, or K. In some embodiments, the method comprises providing an antibody into which the substitution(s) discussed above are introduced. In some embodiments, the method comprises providing an antibody (such as a bispecific or multispecific antibody) that binds one (or more) exemplary targets described elsewhere herein.

Preferential pairing describes the pairing pattern of a first polypeptide (such as a heavy chain) with a second polypeptide (such as a light chain) when one or more additional, distinct polypeptides (e.g., additional heavy chain(s) and/or light chain(s)) are present at the same time as the pairing occurs between the first and second polypeptide. In some embodiments, preferential pairing occurs between, e.g., HC₁ and LC₁ of an antibody (e.g., a bispecific antibody), if the amount of the HC₁/LC₁ heavy chain-light chain pairing is greater than the amount of the HC₁/LC₂ pairing when HC₁ is co-expressed with at least LC₁ and LC₂. Likewise, preferential pairing occurs between, e.g., HC₂ and LC₂ of a multispecific antibody (e.g., a bispecific antibody), if the amount of the HC₂/LC₂ heavy chain-light chain pairing was greater than the amount of the HC₂/LC₁ pairing when HC₂ is co-expressed with at least LC₁, and LC₂. HC₁/LC₁, HC₁/LC₂, HC₂/LC₁, and HC₂/LC₂ pairing can be measured by methods known in the art, e.g., liquid chromatography mass spectrometry (LCMS), as described in further detail elsewhere herein.

In some embodiments the term “original amino acid” refers to the amino acid present at a specific position, e.g., position 94, and/or position 96 of the V_{L}, immediately prior to the substitution , e.g., with a charged amino acid (such as D, R, E, or K). In some embodiments, the term “non-charged amino acid” or “non-charged residue” refers to an amino acid that is neither positively charged (such as protonated) nor negatively charged (such as deprotonated) at a physiological pH, e.g., a pH between about 6.8 and about 7.5, between about 6.9 and about 7.355, or between about 6.95 and 7.45. In some embodiments, a “charged amino acid” refers to an amino acid that is positively charged (such as protonated) or negatively charged (such as deprotonated) at a physiological pH, e.g., a pH between about 6.8 and about 7.5, between about 6.9 and about 7.355, or between about 6.95 and 7.45. In some embodiments, a non-charged amino acid residue is an amino acid residue that is not D, R, E, or K. In some embodiments, the amino acid (e.g., original amino acid) at position 94 is substituted with D. In some embodiments, the amino acid (e.g., original amino acid) at position 96 is substituted with R. In some embodiments, the amino acid (e.g., original amino acid) at position 94 is substituted with D, and the amino acid (e.g., original amino acid) at position 96 is substituted with R.

In some embodiments, the method further comprises substituting the amino acid (e.g., original amino acid) at position 95 of the heavy chain variable domain (V_{H}) from a non-charged residue to a charged residue selected from aspartic acid (D), arginine (R), glutamic acid (E), and lysine (K), wherein the amino acid numbering is according to Kabat. In some embodiments, the amino acid at position 95 (e.g., the original amino acid) is substituted with D. In some embodiments, the amino acid (e.g., original amino acid) at position 94 of the V_{L} is substituted with D, the amino acid (e.g., original amino acid) at position 96 of the V_{L} is substituted with R, and the amino acid (e.g., original amino acid) at position 95 of the V_{H} is substituted with D.

Also provided is a method of improving the pairing (such as cognate pairing, i.e., preferential pairing of cognate V_{H} and V_{L}, Fab, and HC and LC) of a heavy chain and a light chain of an antibody that comprises the step of substituting the amino acid (e.g., original amino acid) at position 95 of the heavy chain variable domain (V_{H}) from a non-charged residue to a charged residue selected from aspartic acid (D), arginine (R), glutamic acid (E), and lysine (K), wherein the amino acid numbering is according to Kabat. In some embodiments, the amino acid at position 95 (e.g., the original amino acid) is substituted with D.

Also provided herein is a method of improving the pairing (such as cognate pairing) of a heavy chain and a light chain of an antibody that comprises the step of substituting at least one amino acid (e.g., “original amino acid”) at position 91 of the light chain variable domain (V_{L})., position 94 of the V_{L}, or position 96 of the V_{L} from a non-aromatic residue to an aromatic residue selected from tryptophan (W), phenylalanine (F) and tyrosine (Y), wherein the amino acid numbering is according to Kabat. In some embodiments, the method comprises the step of substituting at least two amino acids (e.g. original amino acids) at position 91, position 94, or position 96 from non-aromatic residue to an aromatic residue selected from W, F, and Y. In some embodiments, the method comprises the step of substituting the amino acids (e.g., original amino acids) at position 94 and position 96 from a non-aromatic residue to an aromatic residue selected from W, F, and Y. In some embodiments, the method comprises the step of substituting each of the amino acids (e.g., original amino acids) at position 91, position 94, and position 96 from a non-aromatic residue to an aromatic residue selected from W, F, and Y. In some embodiments, the method comprises providing an antibody into which the substitution(s) discussed above are introduced. In some embodiments, the method comprises providing an antibody (such as a bispecific or multispecific antibody) that binds one (or more) exemplary targets described elsewhere herein.

In some embodiments, “original amino acid” refers to the amino acid (e.g., non-aromatic amino acid) present at position 91, position 94, and/or position 96 of the V_{L} immediately prior to the substitution with an aromatic amino acid (e.g., W, F, and Y). In some embodiments, the term “non-aromatic amino acid” or “non-aromatic residue” refers to an amino acid that does not comprise an aromatic ring. In some embodiments, a “non-aromatic residue” refers to an amino acid residue that is not W, F, or Y.

In some embodiments, the amino acid (e.g., original amino acid) at position 91 is substituted with Y. In some embodiments, the amino acid (e.g., original amino acid) at position 94 is substituted with Y. In some embodiments, the amino acid (e.g., original amino acid) at position 96 is substituted with W. In some embodiments, the amino acid (e.g., original amino acid) at position 91 is substituted with Y, and the amino acid (e.g., original amino acid) at position 94 is substituted with Y. In some embodiments, the amino acid (e.g., original amino acid) at position 91 is substituted with Y and the amino acid (e.g., original amino acid) at position 96 is substituted with W. In some embodiments, the amino acid (e.g., original amino acid) at position 94 is substituted with Y, and the amino acid (e.g., original amino acid) at position 96 is substituted with W. In some embodiments, the amino acid (e.g., original amino acid) at position 91 is substituted with Y, the amino acid (e.g., original amino acid) at position 94 is substituted with Y, and the amino acid (e.g., original amino acid) at position 96 is substituted with W.

In some embodiments, the method further comprises substituting the amino acid (e.g., original amino acid) at position 95 of the heavy chain variable domain (V_{H}) from a non-charged residue to a charged residue selected from aspartic acid (D), arginine (R), glutamic acid (E), and lysine (K), wherein the amino acid numbering is according to Kabat. In some embodiments, the method further comprises substituting the amino acid (e.g., original amino acid) at position 95 of the heavy chain variable domain (V_{H}) from a non-aromatic residue to an aromatic residue selected from tryptophan (W), phenylalanine (F) and tyrosine (Y).

In some embodiments, the one or more substitutions described above are introduced into an antibody fragment, e.g., an antibody fragment that comprises a V_{L} domain and a V_{H} domain. Such antibody fragments include, but are not limited to, e.g., a Fab, a Fab′, a monospecific F(ab′)₂, a bispecific F(ab′)₂, a one-armed antibody, an ScFv, an Fv, etc.

In some embodiments, the antibody into which the one or more substitutions described above are introduced is a human, humanized, or chimeric antibody. In some embodiments, the antibody comprises a kappa light chain. In some embodiments, the antibody comprises a lambda light chain. I In certain embodiments, the V_{L} comprises the framework sequences of a KV1 or KV4 human germline family. In some embodiments, the V_{H} comprises the framework sequences of HV2 or HV3 human germline family. In some embodiments, the antibody comprises a murine Fc region. In some embodiments, the antibody comprises a human Fc region, such as a human IgG Fc region, e.g., a human IgG1, human IgG2, human IgG3m or human IgG4 Fc region. In some embodiments, the antibody is a monospecific antibody. In some embodiments, the antibody is a multispecific antibody, e.g., a bispecific antibody.

In certain embodiments, the antibody into which the one or more substitutions described above are introduced is a bispecific antibody that comprises a first V_{L} (V_{L}1) that pairs with a first V_{H} (V_{L}1) and a second V_{L} (V_{L}2) that pairs with a second V_{H} (V_{H}2), wherein V_{L}1 comprises a Q38K substitution mutation, the V_{H}1 comprises a Q39E substitution mutation, V_{L}2 comprises a Q38E substitution mutation, the V_{H}2 comprises a Q39K substitution mutation, wherein amino acid numbering is according to Kabat. In some embodiments, V_{L}1 comprises a Q38E substitution mutation, the V_{H}1 comprises a Q39K substitution mutation, V_{L}2 comprises a Q38K substitution mutation, the V_{H}2 comprises a Q39E substitution mutation, wherein amino acid numbering is according to Kabat. It will be apparent to those of ordinary skill in the art that the terms “V_{L}1,” “V_{H}1,” “V_{L}2,” and “V_{H}2,” are arbitrary designations, and that, e.g., “V_{L}1” and “V_{L}2” in any of the embodiments herein can be reversed.

Additionally or alternatively, in some embodiments, the antibody into which the one or more substitutions described above are introduced is a bispecific antibody that comprises a first heavy chain (HC₁) comprising a first C_{H}1 domain (C_{H}1₁), a first light chain (LC₁) comprising a first C_{L} domain (C_{L1}), a second heavy chain (HC₂) comprising a second C_{H}1 domain (C_{H}1₂), and a second light chain (LC₂) comprising a first CL domain (CL₂). It will be apparent to those of ordinary skill in the art that the terms “HC₁,” “HC₂,” “LC₁,” “LC₂,” etc. are arbitrary designations, and that, e.g., “HC₁” and “HC₂” in any of the embodiments herein can be reversed. That is, any of the mutations above described as being in the C_{H}1 domain of H1 and C_{L} domain of L1 can, alternatively, be in the C_{H}1 domain of H2 and the C_{L} domain of L2. In some embodiments, the method further comprises substituting S183 in C_{H}1₁ with E, V133 in C_{L1} with K, S183 in C_{H}1₂ with K, and V133 in C_{L2} with E, wherein amino acid numbering is according to the EU index. In some embodiments, the method further comprises substituting S183 in CH_{H}1₁ with K, V133 in C_{L1} with E, S183 in C_{H}1₂ with E, and V133 in C_{L2} with K, wherein amino acid numbering is according to the EU index. See, e.g., Dillon et al. (2017) MABS 9(2): 213-230 and WO2016/172485. In some embodiments, HC₁ further comprises a first C_{H}2 (C_{H}2₁) domain and/or a first C_{H}3 (C_{H}3₁) domain. Additionally or alternatively, in some embodiments, HC₂ further comprises a second C_{H}2 (C_{H}2₂) domain and/or a second C_{H}3 (C_{H}3₂) domain. In some embodiments, C_{H}3₂ is altered so that within the C_{H}3₁/C_{H}3₂ interface, one or more amino acid residues are replaced with one or more amino acid residues having a larger side chain volume, thereby generating a protuberance on the surface of C_{H}3₂ that interacts with C_{H}3₁ and C_{H}3₁ is altered so that within the C_{H}3₁/C_{H}3₂ interface, one or more amino acid residues are replaced amino acid residues having a smaller side chain volume, thereby generating a cavity on the surface of C_{H}3₁ that interacts with C_{H}3₂. In some embodiments, C_{H}3₁ is altered so that within the C_{H}3₁/C_{H}3₂ interface, one or more amino acid residues are replaced with one or more amino acid residues having a larger side chain volume, thereby generating a protuberance on the surface of C_{H}3₁ that interacts with C_{H}3₂ and C_{H}3₂ is altered so that within the C_{H}3₁/C_{H}3₂ interface, one or more amino acid residues are replaced amino acid residues having a smaller side chain volume, thereby generating a cavity on the surface of C_{H}3₂ that interacts with C_{H}3₁. In some embodiments, the protuberance is a knob mutation, e.g., a knob mutation that comprises T366W, wherein the amino acid numbering is according to the EU index. In some embodiments, the cavity is a hole mutation, e.g., a hole mutation comprising at least one, at least two, or all three of T366S, L368A, and Y407V, wherein amino acid numbering is according to the EU index. Additional details regarding knob-in-hole mutations are provided in, e.g., U.S. Pat. Nos. 5,731,168, 5,807,706, 7,183,076, the contents of which are incorporated herein by reference in their entireties. In some embodiments, the HC₁/LC₁ pair of the bispecific antibody binds to a first antigen, and the HC₂/LC₂ pair of the bispecific antibody binds to a second antigen. In some embodiments, the HC₁/LC₁ pair of the bispecific antibody binds to a first epitope of a first antigen, and the HC₂/LC₂ pair of the bispecific antibody binds to a second epitope of the first antigen.

Provided is a method of making (such as modifying or engineering) an antibody (such as a bispecific antibody) to obtain a modified antibody (e.g. a modified bispecific antibody) with improved preferential heavy chain/light chain pairing that comprises substituting the amino acid (e.g., original amino acid) at position 94 of the light chain variable domain (V_{L}) and/or position 96 of the V_{L} from a non-charged residue to a charged residue selected from aspartic acid (D), arginine (R), glutamic acid (E), and lysine (K), to obtain the modified antibody (e.g., modified bispecific antibody) wherein the amino acid numbering is according to Kabat. In some embodiments, the method comprises the step of substituting at least both amino acids (e.g. original amino acids) at position 94 and position 96 from non-charged residue to a charged residue, e.g., D, R, E, or K, to obtain the modified antibody (e.g., bispecific antibody). In some embodiments the antibody (e.g., bispecific or multispecific antibody) that is modified binds to an exemplary target described elsewhere herein. In many cases, the sequences of the heavy chains and light chains of antibodies that bind to such targets are publicly available and can be aligned and mapped to the Kabat numbering scheme and then scanned against a Kabat sequence database to identify the position(s) to be substituted.

In some embodiments, the amino acid (e.g., original amino acid) at position 94 is substituted with D to obtain the modified antibody (e.g., modified bispecific antibody). In some embodiments, the amino acid (e.g., original amino acid) at position 96 is substituted with R to obtain the modified antibody (e.g., modified bispecific antibody). In some embodiments, the amino acid (e.g., original amino acid) at position 94 is substituted with D, and the amino acid (e.g., original amino acid) at position 96 is substituted with R to obtain the modified antibody (e.g., modified bispecific antibody).

In some embodiments, the method further comprises substituting the amino acid (e.g., original amino acid) at position 95 of the heavy chain variable domain (V_{H}) from a non-charged residue to a charged residue selected from aspartic acid (D), arginine (R), glutamic acid (E), and lysine (K), to obtain the modified antibody (e.g., modified bispecific antibody), wherein the amino acid numbering is according to Kabat. In some embodiments, the amino acid at position 95 (e.g., the original amino acid) is substituted with D to obtain the modified antibody (e.g., modified bispecific antibody). In some embodiments, the amino acid (e.g., original amino acid) at position 94 of the V_{L} is substituted with D, the amino acid (e.g., original amino acid) at position 96 of the V_{L} is substituted with R, and the amino acid (e.g., original amino acid) at position 95 of the V_{H} is substituted with D to obtain the modified antibody (e.g., modified bispecific antibody).

Also provided is a method of making (such as modifying or engineering) an antibody (such as a bispecific antibody) to obtain a modified antibody (e.g. a modified bispecific antibody) with improved preferential heavy chain/light chain pairing that comprises substituting the amino acid (e.g., original amino acid) at position 95 of the heavy chain variable domain (V_{H}) from a non-charged residue to a charged residue selected from aspartic acid (D), arginine (R), glutamic acid (E), and lysine (K), to obtain the modified antibody (e.g., modified bispecific antibody) wherein the amino acid numbering is according to Kabat. In some embodiments, the amino acid at position 95 (e.g., the original amino acid) is substituted with D to obtain the modified antibody (e.g., modified bispecific antibody).

Also provided is a method of making (such as modifying or engineering) an antibody (such as a bispecific antibody) to obtain a modified antibody (e.g. a modified bispecific antibody) with improved preferential heavy chain/light chain pairing that comprises substituting the amino acid (e.g., original amino acid) at position 91 of the light chain variable domain (V_{L}), position 94 of the V_{L}, and/or position 96 of the V_{L} from a non-aromatic residue to an aromatic residue selected from tryptophan (W), phenylalanine (F), and tyrosine (Y) to obtain the modified antibody (e.g., modified bispecific antibody), wherein the amino acid numbering is according to Kabat. In some embodiments, the method comprises the step of substituting at least two amino acids (e.g. original amino acids) at position 91, position 94, or position 96 from non-aromatic residue to an aromatic residue selected from W, F, and Y to obtain the modified antibody (e.g., modified bispecific antibody). In some embodiments, the method comprises the step of substituting the amino acids (e.g., original amino acids) at position 94 and position 96 from a non-aromatic residue to an aromatic residue selected from W, F, and Y to obtain the modified antibody (e.g., modified bispecific antibody). In some embodiments, the method comprises the step of substituting each of the amino acids (e.g., original amino acids) at position 91, position 94, and position 96 from a non-aromatic residue to an aromatic residue selected from W, F, and Y to obtain the modified antibody (e.g., modified bispecific antibody). In some embodiments the antibody (e.g., bispecific or multispecific antibody) that is modified binds to an exemplary target described elsewhere herein.

In some embodiments, the amino acid (e.g., original amino acid) at position 91 is substituted with Y to obtain the modified antibody (e.g., modified bispecific antibody). In some embodiments, the amino acid (e.g., original amino acid) at position 94 is substituted with Y to obtain the modified antibody (e.g., modified bispecific antibody). In some embodiments, the amino acid (e.g., original amino acid) at position 96 is substituted with W to obtain the modified antibody (e.g., modified bispecific antibody). In some embodiments, the amino acid (e.g., original amino acid) at position 91 is substituted with Y, and the amino acid (e.g., original amino acid) at position 94 is substituted with Y to obtain the modified antibody (e.g., modified bispecific antibody). In some embodiments, the amino acid (e.g., original amino acid) at position 91 is substituted with Y and the amino acid (e.g., original amino acid) at position 96 is substituted with W to obtain the modified antibody (e.g., modified bispecific antibody). In some embodiments, the amino acid (e.g., original amino acid) at position 94 is substituted with Y, and the amino acid (e.g., original amino acid) at position 96 is substituted with W to obtain the modified antibody (e.g., modified bispecific antibody). In some embodiments, the amino acid (e.g., original amino acid) at position 91 is substituted with Y, the amino acid (e.g., original amino acid) at position 94 is substituted with Y, and the amino acid (e.g., original amino acid) at position 96 is substituted with W to obtain the modified antibody (e.g., modified bispecific antibody).

In some embodiments, the method further comprises substituting the amino acid (e.g., original amino acid) at position 95 of the heavy chain variable domain (V_{H}) from a non-charged residue to a charged residue selected from aspartic acid (D), arginine (R), glutamic acid (E), and lysine (K), to obtain the modified antibody (e.g., modified bispecific antibody), wherein the amino acid numbering is according to Kabat. In some embodiments, the method further comprises substituting the amino acid (e.g., original amino acid) at position 95 of the heavy chain variable domain (V_{H}) from a non-aromatic residue to an aromatic residue selected from tryptophan (W), phenylalanine (F), and tyrosine (Y) to obtain the modified antibody (e.g., modified bispecific antibody).

In some embodiments, the method of making (such as modifying or engineering) an antibody (such as a bispecific antibody) comprises modifying a V_{H} and/or a V_{L}, e.g.. by introducing one or more of the substitutions discussed above, into the V_{H} and/or V_{L} to obtain a modified V_{H} and/or modified V_{L}, and grafting modified V_{H} and/or modified V_{L} onto an antibody (such as a bispecific antibody) to obtain the modified antibody (e.g., modified bispecific antibody).

In some embodiments, a V_{H}/V_{L} pair that has been substituted, modified, and/or engineered according to a method described herein is subjected to at least one affinity maturation step (e.g., 1, 2, 3, 4, 5, 6, 7, 8, 9, 10, or more than 10 affinity maturation steps). Affinity maturation is a process by which a heavy chain/light chain pair of, e.g., an antibody obtained by a method described herein, is subject to a scheme that selects for increased affinity for a target (e.g., target ligand or target antigen, as described in further detail below) (see Wu et al. (1998) Proc Natl Acad Sci USA. 95, 6037-42). Details regarding affinity maturation of antibodies are also detailed in, e.g., Merchant et al. (2013) Proc Natl Acad Sci USA 110(32): E2987-96; Julian et al. (2017) Scientific Reports. 7: 45259; Tiller et al. (2017) Front. Immunol. 8: 986; Koenig et al. (2017) Proc Natl Acad Sci USA. 114(4): E486-E495; Yamashita et al. (2019) Structure. 27, 519-527; Payandeh et al. (2019) J Cell Biochem. 120: 940-950; Richter et al. (2019) mAbs. 11(1): 166-177; and Cisneros et al. (2019) Mol. Syst. Des. Eng. 4: 737-746. In certain embodiments, one or more amino acid positions in the V_{H} and/or V_{L} of a heavy chain/light chain pair obtained by a method herein are randomized (i.e., at positions other than those noted above, namely, positions 91, 94, and/or 96 in the V_{L}, and, optionally, position 95 in the V_{H}) to produce a library of heavy chain/light chain variants. The library of V_{H}/V_{L} variants is then screened to identify those variants with the desired affinity for the target. Thus, in certain embodiments, the methods described herein further comprise the steps of (a) mutagenizing or randomizing the CDR-H1, CDR-H2, CDR-H3, CDR-L1, CDR-L2, and/or CDR-L3 of a heavy chain/light chain pair obtained by a method herein at one or more positions to produce a library of V_{H}/V_{L} variants, (b) contacting the library of V_{H}/V_{L} variants with a target (e.g., a target ligand or target antigen), (c) detecting the binding of the target to a V_{H}/V_{L} variant, and (d) obtaining the V_{H}/V_{L} variant that specifically binds the target. As noted above, positions 91, 94, and/or 96 in the V_{L} and, optionally, position 95 in the V_{H} in the antigen binding domain variant are not targeted for further randomization. The methods for mutagenizing CDR-H1, CDR-H2, CDR-H3, CDR-L1, CDR-L2, and/or CDR-L3 of an antibody (or fragment antigen-binding fragment thereof) are known in the art, and discussed elsewhere herein. Details regarding libraries and library screens are provided elsewhere herein.

In certain embodiments, the methods described herein further comprise a step of (e) determining the nucleic acid sequence of the V_{H}/V_{L} variant (i.e., the affinity matured V_{H}/V_{L} pair) that specifically binds the target. In some embodiments, the methods described herein further comprise the step of (f) grafting the affinity matured V_{H}/V_{L} pair onto an antibody (such as a bispecific antibody) to an affinity matured, modified antibody (e.g., affinity matured, modified bispecific antibody). In some embodiments, the methods describe herein further comprise the step of (g) assessing the degree to which the affinity matured V_{H}/V_{L} pair demonstrates preferential pairing/preferential assembly, e.g., using a method described below.

Also provided herein is an antibody (e.g., a monospecific, bispecific, or multispecific antibody) or an antibody fragment produced according to any one or combination of methods described above.

Preferential Pairing/Preferential Assembly of Antibody Heavy Chains and Light Chains

As noted above, preferential pairing describes the pairing pattern of a first polypeptide (such as a heavy chain) with a second polypeptide (such as a light chain) when one or more additional, distinct polypeptides (e.g., additional heavy chain(s) and/or light chain(s)) are present at the same time as the pairing occurs between the first and second polypeptide. Preferential pairing (e.g., cognate pairing) occurs between, e.g., HC₁ and LC₁ of an antibody (e.g., a bispecific antibody), if the amount of the HC₁/LC₁ heavy chain-light chain pairing is greater than the amount of the HC₁/LC₂ pairing when HC₁ is co-expressed with at least LC₁ and LC₂. Likewise, preferential pairing (e.g., cognate pairing) occurs between, e.g., HC₂ and LC₂ of a multispecific antibody (e.g., a bispecific antibody), if the amount of the HC₂/LC₂ heavy chain-light chain pairing was greater than the amount of the HC₂/LC₁ pairing when HC₂ is co-expressed with at least LC₁, and LC₂. HC₁/LC₁, HC₁/LC₂, HC₂/LC₁, and HC₂/LC₂ pairing can be measured by methods known in the art, e.g., liquid chromatography mass spectrometry (LCMS), as described in further detail elsewhere herein.

In certain embodiments, the methods provided herein are used to generate (such as produce) an antibody (e.g., a bispecific antibody) in which HC₁ preferentially pairs with the LC₁. Additionally or alternatively, the methods provided herein are used to generate (such as produce) an antibody (e.g., a bispecific antibody) in which the HC₂ preferentially pairs with the LC₂. In certain embodiments, the methods provided herein are used to generate (such as produce) an antibody (e.g., a bispecific antibody) in which HC₁ preferentially pairs with the LC₁ and the HC₂ preferentially pairs with the LC₂. In certain embodiments, when an HC₁ of an antibody (e.g., a bispecific antibody) generated by a method provided herein is co-expressed with HC₂, LC₁, and LC₂, a bispecific antibody comprising the desired pairings (e.g., HC₁/LC₁ and HC₂/LC₂) is produced with a relative yield of at least about 30%, at least about 35%, at least about 40%, at least about 45%, at least about 50%, at least about 55%, at least about 60%, at least about 70%, at least about 71%, at least about 71%, at least about 72%, at least about 73%, at least about 74% , at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 99%, or more than about 99%, including any range in between these values. The relative yield of bispecific antibody comprising the desired pairings (e.g., HC₁/LC₁ and HC₂/LC₂) can be determined using, e.g., mass spectrometry, as described in the Examples.

In certain embodiments, the expressed polypeptides of an antibody (such as a bispecific antibody) generated using a method provided herein assemble with improved specificity to reduce generation of mispaired heavy chains and light chains. In certain embodiments, the V_{H} domain of C_{H}1 of an antibody (e.g., bispecific antibody) provided herein assembles (such as preferentially assembles) with the V_{L} domain of LC₁ during production.

Methods of Assessing Correct Pairing /Preferential Pairing /Preferential Assembly

Preferential pairing, correct pairing, and/or preferential assembly of the HC₁ with the LC₁ of a modified antibody (e.g., a modified bispecific antibody) made according to a method described herein can be determined using any one of a variety of methods well known to those of ordinary skill in the art. For example, the degree of preferential pairing of the HC₁ with LC₁ in a modified antibody (such as a modified bispecific antibody) can be determined via Light Chain Competition Assay (LCCA). International patent application PCT/US2013/063306, filed Oct. 3, 2013, describes various embodiments of LCCA and is herein incorporated by reference in its entirety for all purposes. The method allows quantitative analysis of the pairing of heavy chains with specific light chains within the mixture of co-expressed proteins and can be used to determine if one particular immunoglobulin heavy chain selectively associates with either one of two immunoglobulin light chains when the heavy chain and light chains are co-expressed. The method is briefly described as follows: At least one heavy chain and two different light chains are co-expressed in a cell, in ratios such that the heavy chain is the limiting pairing reactant; optionally separating the secreted proteins from the cell; separating the immunoglobulin light chain polypeptides bound to heavy chain from the rest of the secreted proteins to produce an isolated heavy chain paired fraction; detecting the amount of each different light chain in the isolated heavy chain fraction; and analyzing the relative amount of each different light chain in the isolated heavy chain fraction to determine the ability of the at least one heavy chain to selectively pair with one of the light chains.

In certain embodiments, preferential pairing of the HC₁ with the LC₁ of a modified antibody (e.g., a modified bispecific or multispecific antibody) made according to a method provided herein is measured via mass spectrometry (such as liquid chromatography-mass spectrometry (LC-MS) native mass spectrometry, acidic mass spectrometry, etc.). Mass spectrometry is used to quantify the relative heterodimer populations including each light chain using differences in their molecular weight to identify each distinct species. In certain embodiments, correct or preferential pairing is determined by LC-MS as described herein. In certain embodiments, correct or preferential pairing of Fv or Fab is measured.

Multispecific Antibody Formats

A modified antibody (such as a modified bispecific antibody) made according to a method provided herein can be used with any one of a variety of bispecific or multispecific antibody formats known in the art. Numerous formats have been developed in the art to address therapeutic opportunities afforded by molecules with multiple binding specificities. Several approaches have been described to prepare bispecific antibodies in which specific antibody light chains or fragment pair with specific antibody heavy chains or fragments.

For example, mutations in the C_{H}1/C_{L} interface that facilitate selective pairing of cognate Fab or HC and LC pairing are described in Dillon et al. (2017) MABS 9(2): 213-230 and WO2016/172485, the contents of which are incorporated herein by reference in their entirety.

Knob-into-hole is a heterodimerization technology for the C_{H}3 domain of an antibody. Previously, knobs-into-holes technology has been applied to the production of human full-length bispecific antibodies with a single common light chain (LC) (Merchant et al. “An efficient route to human bispecific IgG.” Nat Biotechnol. 1998; 16:677-81; Jackman et al. “Development of a two-part strategy to identify a therapeutic human bispecific antibody that inhibits IgE receptor signaling.” J Biol Chem. 2010;285:20850-9.) See also WO1996027011, which is herein incorporated by reference in its entirety for all purposes.

An antibody (such as bispecific antibody) generated using a method provided herein can be further modified to comprise other heterodimerization domain(s) having a strong preference for forming heterodimers over homodimers. Illustrative examples include but are not limited to, for example, WO2007147901 (Kjærgaard et al.—Novo Nordisk: describing ionic interactions); WO 2009089004 (Kalman et al.—Amgen: describing electrostatic steering effects); WO 2010/034605 (Christensen et al.—Genentech; describing coiled coils). See also, for example, Pack, P. & Plückthun, A., Biochemistry 31, 1579-1584 (1992) describing leucine zipper or Pack et al., Bio/Technology 11, 1271-1277 (1993) describing the helix-turn-helix motif. The phrase “heteromultimerization domain” and “heterodimerization domain” are used interchangeably herein. In certain embodiments, an antibody (such as bispecific antibody) produced using a method provided herein comprises one or more heterodimerization domains.

US Patent Publication No. 2009/0182127 (Novo Nordisk, Inc.) describes the generation of bi-specific antibodies by modifying amino acid residues at the Fc interface and at the C_{H}1:C_{L} interface of light-heavy chain pairs that reduce the ability of the light chain of one pair to interact with the heavy chain of the other pair.

Techniques for making multispecific antibodies include, but are not limited to, recombinant co-expression of two immunoglobulin heavy chain-light chain pairs having different specificities (see Milstein and Cuello, Nature 305: 537 (1983)) and “knob-in-hole” engineering (see, e.g., U.S. Pat. No. 5,731,168, and Atwell et al., J. Mol. Biol. 270:26-35 (1997)). Multi-specific antibodies may also be made by engineering electrostatic steering effects for making antibody Fc-heterodimeric molecules (see, e.g., WO 2009/089004); cross-linking two or more antibodies or fragments (see, e.g., U.S. Pat. No. 4,676,980, and Brennan et al., Science, 229: 81 (1985)); and using leucine zippers to produce bi-specific antibodies (see, e.g., Kostelny et al., J. Immunol., 148(5):1547-1553 (1992) and WO 2011/034605).

Multi-specific antibodies may also be provided in an asymmetric form with a domain crossover in one or more binding arms of the same antigen specificity, i.e. by exchanging the V_{H}/V_{L} domains (see e.g., WO 2009/080252 and WO 2015/150447), the C_{H}1/C) domains (see e.g., WO 2009/080253) or the complete Fab arms (see e.g., WO 2009/080251, WO 2016/016299, also see Schaefer et al, PNAS, 108 (2011) 1187-1191, and Klein at al., MAbs 8 (2016) 1010-20). In one aspect, the multispecific antibody comprises a cross-Fab fragment. The term “cross-Fab fragment” or “xFab fragment” or “crossover Fab fragment” refers to a Fab fragment, wherein either the variable regions or the constant regions of the heavy and light chain are exchanged. A cross-Fab fragment comprises a polypeptide chain composed of the light chain variable region (V_{L}) and the heavy chain constant region 1 (C_{H}1), and a polypeptide chain composed of the heavy chain variable region (V_{H}) and the light chain constant region (C_{L}). Asymmetrical Fab arms can also be engineered by introducing charged or non-charged amino acid mutations into domain interfaces to direct correct Fab pairing. See e.g., WO 2016/172485.

Reviews of various bispecific and multispecific antibody formats are provided in Klein et al., (2012) mAbs 4:6, 653-663 and Spiess et al. (2015) “Alternative molecular formats and therapeutic applications for bispecific antibodies.”Mol. Immunol. 67 (2015) 95-106.

In some embodiments, a modified antibody (e.g., a modified bispecific antibody) made by a method provided herein is reformatted into any of the multispecific antibody formats described above to further ensure correct heavy/light chain pairing.

### Production and Purification of Antibodies

Culturing Host Cells

In certain embodiments, an modified antibody (such as a modified bispecific or multispecific antibody) made according to a method provided herein can be produced by (a) introducing a set of polynucleotides encoding HC₁, HC₂, LC₁, and LC₂ into a host cell; and (b) culturing the host cell to produce the antibody (e.g., bispecific or multispecific antibody). In certain embodiments, the polynucleotides encoding LC₁ and LC₂ are introduced into the host cell at a predetermined ratio (e.g., a molar ratio or a weight ratio). In certain embodiments, polynucleotides encoding LC₁ and LC₂ are introduced into the host cell such that the ratio (e.g., a molar ratio or a weight ratio) of LC₁:LC₂ is about 1:1, about 1:1.5, about 1:2, about 1:2.5, about 1:3, about 1:3.5, about 1:4, about 1:4.5, about 1:5, about 1:5.5, about 1.5:1, about 2:1, about 2.5:1, about 3:1, about 3.5:1, about 4:1, about 4.5:1, about 5:1, or about 5.5:1, including any range in between these values. In certain embodiments, the ratio is a molar ratio. In certain embodiments the ratio is a weight ratio. In certain embodiments, the polynucleotides encoding HC₁ and HC₂ are introduced into the host cell at a predetermined ratio (e.g., a molar ratio or a weight ratio). In certain embodiments, polynucleotides encoding HC₁ and HC₂ are introduced into the host cell such that the ratio (e.g., a molar ratio or a weight ratio) of HC₁:HC₂ is about 1:1, about 1:1.5, about 1:2, about 1:2.5, about 1:3, about 1:3.5, about 1:4, about 1:4.5, about 1:5, about 1:5.5, about 1.5:1, about 2:1, about 2.5:1, about 3:1, about 3.5:1, about 4:1, about 4.5:1, about 5:1, or about 5.5:1, including any range in between these values. In certain embodiments, the ratio is molar ratio. In certain embodiments the ratio is a weight ratio. In certain embodiments, the polynucleotides encoding HC₁, HC₂, LC₁, and LC₂ are introduced into the host cell at a predetermined ratio (e.g., a molar ratio or a weight ratio). In certain embodiments, polynucleotides encoding HC₁, HC₂, LC₁, and LC₂ are introduced into the host cell such that the ratio (e.g., a molar ratio or a weight ratio) of HC₁+HC₂:LC₁, +LC₂ is about 5:1, about 5:2, about 5:3, about 5:4, about 1:1, about 4:5, about 3:5, about 2:5, or about 1:5, including any range in between these values. In certain embodiments, polynucleotides encoding LC₁, LC₂, HC₁, and HC₂ are introduced into the host cell such that the ratio (e.g., a molar ratio or a weight ratio) of LC₁+LC₂:HC₁, +HC₂ is about 1:1:1:1, about 2.8:1:1:1, about 1.4:1:1:1, about 1:1.4:1:1, about 1:2.8:1:1, about 1:1:2.8:1, about 1:1:1.4:1, about 1:1:1:2.8, or about 1: 1:1:1.4, including any range in between these values. In certain embodiments, the ratio is molar ratio. In certain embodiments the ratio is a weight ratio.

In certain embodiments, producing a modified antibody (such as a modified bispecific or multispecific antibody) made according to a method provided herein further comprises determining an optimal ratio of the polynucleotides for introduction into the cell. In certain embodiments, mass spectrometry is used to determine antibody yield (such as bispecific antibody yield), and optimal chain ratio is adjusted to maximize protein yield (such as bispecific antibody yield). In certain embodiments, producing an antibody (such as a bispecific or multispecific antibody) generated according to a method provided herein further comprises harvesting or recovering the antibody from the cell culture. In certain embodiments, producing an antibody (such as a bispecific or multispecific antibody) generated according to a method provided herein further comprises purifying the harvested or recovered antibody.

The host cells used to produce a modified antibody (such as modified bispecific antibody) made according to a method provided herein may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem.102:255 (1980), U.S. Pat. Nos. 4,767,704; 4,657,866; 4,927,762; 4,560,655; or 5,122,469; WO 90/03430; WO 87/00195; or U.S. Pat. No. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

Harvesting or Recovering and Purifying Antibodies

In a related aspect, producing a modified antibody (such as a modified bispecific antibody) made according to a method described herein comprises culturing a host cell described above under conditions that allow expression of the modified antibody and recovering (such as harvesting) the modified antibody. In certain embodiments, producing a modified antibody (such as a modified bispecific antibody) made according to a method described herein further comprises purifying the recovered modified antibody (such as a modified bispecific antibody) to obtain a preparation that is substantially homogeneous, e.g., for further assays and uses.

A modified antibody (such as a modified bispecific antibody) made according to a method described herein can be produced intracellularly, or directly secreted into the medium. If such modified antibody is produced intracellularly, as a first step, the particulate debris, either host cells or lysed fragments, are removed, for example, by centrifugation or ultrafiltration. Where the modified antibody (such as a modified bispecific antibody) made according to a method described herein is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

Standard protein purification methods known in the art can be employed to obtain substantially homogeneous preparations of a modified antibody (such as a modified bispecific antibody) made according to a method described herein from cells. The following procedures are exemplary of suitable purification procedures: fractionation on immunoaffinity or ion-exchange columns, ethanol precipitation, reverse phase HPLC, chromatography on silica or on a cation-exchange resin such as DEAE, chromatofocusing, SDS-PAGE, ammonium sulfate precipitation, and gel filtration using, for example, Sephadex G-75.

Additionally or alternatively, a modified antibody (such as a modified bispecific antibody) made using a method described herein can be purified using, for example, hydroxyapatite chromatography, gel electrophoresis, dialysis, and affinity chromatography, with affinity chromatography being the preferred purification technique.

In certain aspects, the preparation derived from the cell culture medium as described above is applied onto the Protein A immobilized solid phase to allow specific binding of the modified antibody (such as a modified bispecific antibody) to protein A. The solid phase is then washed to remove contaminants non-specifically bound to the solid phase. The modified antibody (such as a modified bispecific antibody) is recovered from the solid phase by elution into a solution containing a chaotropic agent or mild detergent. Exemplary chaotropic agents and mild detergents include, but are not limited to, Guanidine-HCl, urea, lithium perclorate, arginine, histidine, SDS (sodium dodecyl sulfate), Tween, Triton, and NP-40, all of which are commercially available.

The suitability of protein A as an affinity ligand depends on the species and isotype of any immunoglobulin Fc domain that is present in the antibody (such as bispecific antibody). Protein A can be used to purify antibodies that are based on human γ1, γ2, or γ4 heavy chains (Lindmark et al., J. Immunol. Meth. 62:1-13 (1983)). Protein G is recommended for all mouse isotypes and for human γ3 (Guss et al., EMBO J. 5:15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the modified antibody (such as a modified bispecific antibody) comprises a C_{H}3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody (such as bispecific antibody) to be recovered.

Following any preliminary purification step(s), the mixture comprising the modified antibody (such as a modified bispecific antibody) and contaminants may be subjected to low pH hydrophobic interaction chromatography using an elution buffer at a pH between about 2.5-4.5, preferably performed at low salt concentrations (e.g., from about 0-0.25M salt). The production of a modified antibody (such as a modified bispecific antibody) can alternatively or additionally (to any of the foregoing particular methods) comprise dialyzing a solution comprising a mixture of the polypeptides.

Libraries and Library Screens

Also provided herein are libraries of heavy chain/light chain pairs (or antigen binding fragments thereof) that exhibit preferential pairing.

For example, provided herein is a library comprising a plurality of antigen binding domain variants, each antigen binding domain variant comprising a different antibody heavy chain domain (V_{H}) and a different antibody light chain domain (V_{L}), wherein each V_{H} comprises different CDR-H1, CDR-H2, and CDR-H3 sequences, wherein each V_{L} comprises different CDR-L1, CDR-L2, and CDR-L3 sequences, and wherein at least one amino acid at position 94 in each V_{L}, or position 96 of each V_{L} is a charged residue selected from aspartic acid (D), arginine (R), glutamic acid (E), and lysine (K), wherein the amino acid numbering is according to Kabat. In some embodiments, both two amino acids at position 94 and position 96 of each V_{L} is a charged residue independently selected from D, R, E, and K. In some embodiments, the amino acid at position 94 of each V_{L} is D. In some embodiments, the amino acid at position 96 of each V_{L} is R. In some embodiments, the amino acid at position 94 of each V_{L} is D and the amino acid at position 96 of each V_{L} is R. In some embodiments, the amino acid at position 95 of each V_{H} is a charged residue selected from D, R, E, and K. In some embodiments, the amino acid at position 95 of each V_{H} is D. In some embodiments, the amino acid at position 94 of each V_{L} is D, the amino acid at position 96 of each V_{L} is R, and the amino acid at position 95 of each V_{H} is D.

Also provided herein is a library comprising a plurality of antigen binding domain variants, each antigen binding domain variant comprising a different antibody heavy chain domain (V_{H}) and a different antibody light chain domain (V_{L}), wherein each V_{H} comprises different CDR-H1, CDR-H2, and CDR-H3 sequences, wherein each V_{L} comprises different CDR-L1, CDR-L2, and CDR-L3 sequences, and wherein at least one amino acid at position 91 of each V_{L}, position 94 in each V_{L}, or position 96 of each V_{L} is an aromatic residue selected from tryptophan (W), phenylalanine (F), and tyrosine (Y), wherein the amino acid numbering is according to Kabat. In some embodiments, at least two amino acids at position 91, position 94, or position 96 (e.g., positions 91 and 94, positions 91 and 96, or positions 94 and 96) of each V_{L} is an aromatic residue selected from W, F, and Y. In some embodiments, the amino acid at position 91 of each V_{L} is Y. In some embodiments, the amino acid at position 94 of each V_{L} is Y. In some embodiments, the amino acid at position 96 of each V_{L} is W. In some embodiments, the amino acid at position 91 of each V_{L} is Y, and the amino acid at position 94 of each V_{L} is Y. In some embodiments, the amino acid at position 91 of each V_{L} is Y and the amino acid at position 96 of each V_{L} is W. In some embodiments, the amino acid at position 94 of each V_{L} is Y, and the amino acid at position 96 of each V_{L} is W. In some embodiments, the amino acid at position 91 of each V_{L} is Y, the amino acid at position 94 of each V_{L} is Y, and the amino acid at position 96 of each V_{L} is W. In some embodiments, the amino acid at position 95 of each V_{H} is a charged residue selected from aspartic acid (D), arginine (R), glutamic acid (E), and lysine (K), wherein the amino acid numbering is according to Kabat. In some embodiments, the amino acid at position 95 of each V_{H} is an aromatic residue selected from tryptophan (W), phenylalanine (F), and tyrosine (Y).

In certain embodiments, the library is a polypeptide library (such as a plurality of any of the polypeptides described herein). In certain embodiments, a polypeptide library provided herein is a polypeptide display library. Such polypeptide display libraries can be screened to select and/or evolve binding proteins with desired properties for a wide variety of utilities, including but not limited to therapeutic, prophylactic, veterinary, diagnostic, reagent, or material applications. In certain embodiments, the library is a nucleic acid library (such as a plurality of any of the nucleic acids described herein), wherein each nucleic acid (or a group of nucleic acids) encodes a different antigen domain binding variant described herein. In some embodiments, the library is a plurality of host cells (e.g., prokaryotic or eukaryotic host cells) each comprising (and, e.g., expressing) a different nucleic acid (or a group of nucleic acids), wherein each different nucleic acid (or a group of nucleic acids) encodes a different antigen domain binding variant described herein

In certain embodiments, a library provided herein comprises at least 2, 3, 4, 5, 10, 30, 100, 250, 500, 750, 1000, 2500, 5000, 7500, 10000, 25000, 50000, 75000, 100000, 250000, 500000, 750000, 1000000, 2500000, 5000000, 7500000, 10000000, or more than 10000000 different antigen binding domains, including any range in between these values. In certain embodiments, a library provided herein has a sequence diversity of about 2, about 5, about 10, about 50, about 100, about 250, about 500, about 750, about 10³, about 10⁴, about 10⁵, about 10⁶, about 10⁷, about 10⁸, about 10⁹, about 10¹⁰, about 10¹¹, about 10¹², about 10¹³, about 10¹⁴, or more than about 10¹⁴ (such as about 10¹⁵ or about 10¹⁶), including any range in between these values.

In certain embodiments, a library provided herein is generated via genetic engineering. A variety of methods for mutagenesis and subsequent library construction have been previously described (along with appropriate methods for screening or selection). Such mutagenesis methods include, but are not limited to, e.g., error-prone PCR, loop shuffling, or oligonucleotide-directed mutagenesis, random nucleotide insertion or other methods prior to recombination. Further details regarding these methods are described in, e.g., Abou-Nadler et al. (2010) Bioengineered Bugs 1, 337-340; Firth et al. (2005) Bioinformatics 21, 3314-3315; Cirino et al. (2003) Methods Mol Biol 231, 3-9; Pirakitikulr (2010) Protein Sci 19, 2336-2346; Steffens et al. (2007) J Biomol Tech 18, 147-149; and others. Accordingly, in certain embodiments, provided are multispecific antigen-binding protein libraries generated via genetic engineering techniques.

In certain embodiments, a library provided herein is generated via in vitro translation. Briefly, in vitro translation entails cloning the protein-coding sequence(s) into a vector containing a promoter, producing mRNA by transcribing the cloned sequence(s) with an RNA polymerase, and synthesizing the protein by translation of this mRNA in vitro, e.g., using a cell-free extract. A desired mutant protein can be generated simply by altering the cloned protein-coding sequence. Many mRNAs can be translated efficiently in wheat germ extracts or in rabbit reticulocyte lysates. Further details regarding in vitro translation are described in, e.g., Hope et al. (1985) Cell 43, 177-188; Hope et al. (1986) Cell 46, 885-894; Hope et al. (1987) EMBO J. 6, 2781-2784; Hope et al. (1988) Nature 333, 635-640; and Melton et al. (1984) Nucl. Acids Res.12, 7057-7070.

Accordingly, provided is a plurality of nucleic acid molecules encoding a polypeptide display library described herein. An expression vector operably linked to the plurality of nucleic acid molecules is also provided herein. Also provided is a method of making a library provided herein by providing a plurality of nucleic acids encoding a plurality of antigen binding domains described herein, and expressing the nucleic acids.

In certain embodiments, a library provided herein is generated via chemical synthesis. Methods of solid phase and liquid phase peptide synthesis are well known in the art and described in detail in, e.g., Methods of Molecular Biology, 35, Peptide Synthesis Protocols, (M. W. Pennington and B. M. Dunn Eds), Springer, 1994; Welsch et al. (2010) Curr Opin Chem Biol 14, 1-15; Methods of Enzymology, 289, Solid Phase Peptide Synthesis, (G. B. Fields Ed.), Academic Press, 1997; Chemical Approaches to the Synthesis of Peptides and Proteins, (P. Lloyd-Williams, F. Albericio, and E. Giralt Eds), CRC Press, 1997; Fmoc Solid Phase Peptide Synthesis, A Practical Approach, (W. C. Chan, P. D. White Eds), Oxford University Press, 2000; Solid Phase Synthesis, A Practical Guide, (S. F. Kates, F Albericio Eds), Marcel Dekker, 2000; P. Seneci, Solid-Phase Synthesis and Combinatorial Technologies, John Wiley & Sons, 2000; Synthesis of Peptides and Peptidomimetics (M. Goodman, Editor-in-chief, A. Felix, L. Moroder, C. Tmiolo Eds), Thieme, 2002; N. L. Benoiton, Chemistry of Peptide Synthesis, CRC Press, 2005; Methods in Molecular Biology, 298, Peptide Synthesis and Applications, (J. Howl Ed) Humana Press, 2005; and Amino Acids, Peptides and Proteins in Organic Chemistry, Volume 3, Building Blocks, Catalysts and Coupling Chemistry, (A. B. Hughs, Ed.) Wiley-VCH, 2011. Accordingly, in certain embodiments, provided is a multispecific antigen-binding protein library generated via chemical synthesis techniques.

In certain embodiments, a library provided herein is a display library. In certain embodiments, the display library is a phage display library, a phagemid display library, a virus display library, a bacterial display library, a yeast display library, a λgt11 library, a CIS display library, and in vitro compartmentalization library, or a ribosome display library. Methods of making and screening such display libraries are well known to those of skill in the art and described in, e.g., Molek et al. (2011) Molecules 16, 857-887; Boder et al., (1997) Nat Biotechnol 15, 553-557; Scott et al. (1990) Science 249, 386-390; Brisette et al. (2007) Methods Mol Biol 383, 203-213; Kenrick et al. (2010) Protein Eng Des Sel 23, 9-17; Freudl et al. (1986) J Mol Biol 188,491-494; Getz et al. (2012) Methods Enzymol 503, 75-97; Smith et al. (2014) Curr Drug Discov Technol 11, 48-55; Hanes, et al. (1997) Proc Natl Acad Sci USA 94,4937-4942; Lipovsek et al., (2004) J Imm Methods 290, 51-67; Ullman et al. (2011) Brief. Funct. Genomics, 10, 125-134; Odegrip et al. (2004) Proc Natl Acad Sci USA 101, 2806-2810; and Miller et al. (2006) Nat Methods 3, 561-570.

In certain embodiments, a library provided herein is an RNA-protein fusion library generated, for example, by the techniques described in Szostak et al., U.S. Pat. Nos. 6,258,558, 6,261,804, 5,643,768, and 5,658,754. In certain embodiments, a library provided herein is a DNA-protein library, as described, for example, in U.S. Pat. No. 6,416,950.

Methods of Screening

A library provided herein can be screened to identify an antigen binding variant with high affinity for a target (e.g., antigen) of interest. Accordingly, provided herein is a method of obtaining an antigen binding variant that binds a target of interest (e.g., a target of interest described elsewhere herein).

In certain embodiments, the method comprises a) contacting a library described herein under a condition that allows binding of a target of interest with an antigen binding domain variant in the library that specifically binds the target, (b) detecting the binding of the target with the antigen binding domain variant that specifically binds the target (e.g., detecting a complex comprising the target and the antigen binding domain variant that specifically binds the target), and (c) obtaining the antigen binding domain variant that specifically binds the target. In some embodiments, the method further comprises subjecting the antigen binding domain variant thus identified to at least one affinity maturation step, wherein the amino acid at position 91, position 94, and/or position 96 in the V_{L} of the antigen binding domain variant is not selected for randomization. In some embodiments, the amino acid at position 95 in the V_{H} is not selected for randomization.

In some embodiments, the method further comprises producing an antibody (such as a bispecific antibody or a multispecific antibody) that comprises the antigen binding domain variant that binds the target of interest (e.g., an affinity matured antigen binding domain variant that binds the target of interest).

In certain embodiments, provided is a complex comprising a target and an antigen binding domain variant that specifically binds the target. In certain embodiments, the method further comprises determining the nucleic acid sequence(s) of V_{H} and/or V_{L} of the antigen binding domain variant.

Affinity maturation is a process during which an antigen binding domain variant is subject to a scheme that selects for increased affinity for a target (e.g., target ligand or target antigen) (see Wu et al. (1998) Proc Natl Acad Sci USA. 95, 6037-42). In certain embodiments, an antigen binding domain variant that specifically binds a first target ligand is further randomized (i.e., at positions other than those noted above, namely, positions 91, 94, and/or 96 in the V_{L}, and, optionally, position 95 in the V_{H}) after identification from a library screen. For example, in certain embodiments, the method of obtaining an antigen binding domain variant that specifically binds a first target ligand further comprises (e) mutagenizing or randomizing the CDR-H1, CDR-H2, CDR-H3, CDR-L1, CDR-L2, and/or CDR-L3 of the an antigen binding domain variant identified previously to generate further antigen binding domain variants, (f) contacting the first target ligand with the further randomized antigen binding domain variants, (g) detecting the binding of the target to a further randomized antigen binding domain variant, and (h) obtaining a further randomized antigen binding domain variant that specifically binds the target. As noted above, positions 91, 94, and/or 96 in the V_{L} and, optionally, position 95 in the V_{H} in the antigen binding domain variant are not targeted for further randomization. The methods for mutagenizing CDR-H1, CDR-H2, CDR-H3, CDR-L1, CDR-L2, and/or CDR-L3 of the an antigen binding domain are known in the art, and may include, for example, random mutagenesis, CDR walking mutagenesis or sequential and parallel optimization, mutagenesis by structure-based rational design, site-specific mutagenesis, enzyme-based mutagenesis, chemical-based mutagenesis, and gene synthesis methods for synthetic antibody gene production. See, e.g., Yang et al., 1995, CDR Walking Mutagenesis for the Affinity Mutation of a Potent Human Anti-HIV-1 Antibody into the Picomolar Range, J. Mol. Biol. 254:392-40, and Lim et al., 2019, Review: Cognizance of Molecular Methods for the Generation of Mutagenic Phage Display Antibody Libraries for Affinity Maturation, Int. J. Mol. Sci, 20:1861, the contents of which are both incorporated by reference herein in their entireties.

In certain embodiments, the method further comprises (i) determining the nucleic acid sequence of the antigen binding domain variant that specifically binds the target.

In certain embodiments, the further randomized antigen binding domain variants comprise at least one or at least two randomized CDRs which were not previously randomized in the first library. Multiple rounds of randomization (i.e., other than at positions 91, 94, and/or 96 in the V_{L} and, optionally, position 95 in the V_{H}), screening and selection can be performed until antigen binding domain variant(s) having sufficient affinity for the target are obtained. Thus, in certain embodiments, steps (e)-(h) or steps (e)-(i) are repeated one, two, three, four, five, six, seven, eight, nine, ten, or more than ten times in order to identify antigen binding domain variant(s) that specifically binds a first target ligand. In some embodiments, antigen binding domain variant(s) that have undergone two or more rounds of randomization, screening and selection bind the target with affinities that are at least as high as those of antigen binding domain variant(s) that have undergone one round of randomization, screening, and selection.

A library of antigen binding domain variants described herein may be screened by any technique known in the art for evolving new or improved binding proteins that specifically bind a target ligand. In certain embodiments, the target ligand is immobilized on a solid support (such as a column resin or microtiter plate well), and the target ligand is contacted with a library of candidate multispecific antigen-binding proteins (such as any library described herein). Selection techniques can be, for example, phage display (Smith (1985) Science 228, 1315-1317), mRNA display (Wilson et al. (2001) Proc Natl Acad Sci USA 98: 3750-3755) bacterial display (Georgiou, et al. (1997) Nat Biotechnol 15:29-34.), yeast display (Boder and Wittrup (1997) Nat. Biotechnol. 15:553-5577) or ribosome display (Hanes and Plückthun (1997) Proc Natl Acad Sci USA 94:4937-4942 and WO2008/068637).

In certain embodiments, the library of antigen binding domain variants is a phage display library. In certain embodiments, provided is a phage particle displaying an antigen binding domain variant described herein. In certain embodiments, provided is a phage particle displaying an antigen binding domain variant described herein that is capable of binding to a target ligand.

Phage display is a technique by which a plurality of multispecific antigen-binding protein variants are displayed as fusion proteins to the coat protein on the surface of bacteriophage particles (Smith, G. P. (1985) Science, 228:1315-7; Scott, J. K. and Smith, G. P. (1990) Science 249: 386; Sergeeva, A., et al. (2006) Adv. Drug Deliv. Rev. 58:1622-54). The utility of phage display lies in the fact that large libraries of selectively randomized protein variants (or randomly cloned cDNAs) can be rapidly and efficiently sorted for those sequences that bind to a target molecule with high affinity

Display of peptides (Cwirla, S. E. et al. (1990) Proc. Natl. Acad. Sci. USA, 87:6378) or protein (Lowman, H. B. et al. (1991) Biochemistry, 30:10832; Clackson, T. et al. (1991) Nature, 352: 624; Marks, J. D. et al. (1991), J Mol. Biol., 222:581; Kang, A. S. et al. (1991) Proc. Natl. Acad. Sci. USA, 88:8363) libraries on phage have been used for screening millions of polypeptides or oligopeptides for ones with specific binding properties (Smith, G. P. (1991) Current Opin. Biotechnol., 2:668; Wu et al. (1998) Proc Natl Acad Sci USA. May 95, 6037-42). Polyvalent phage display methods have been used for displaying small random peptides and small proteins through fusions to either gene III or gene VIII of filamentous phage. (Wells and Lowman, Curr. Opin. Struct. Biol., 3:355-362 (1992), and references cited therein.) In a monovalent phage display, a protein or peptide library is fused to a gene III or a portion thereof, and expressed at low levels in the presence of wild type gene III protein so that phage particles display one copy or none of the fusion proteins. Avidity effects are reduced relative to polyvalent phage so that sorting is on the basis of intrinsic ligand affinity, and phagemid vectors are used, which simplify DNA manipulations. (Lowman and Wells, Methods: A companion to Methods in Enzymology, 3:205-0216 (1991).)

Sorting phage libraries of antigen binding domain variants entails the construction and propagation of a large number of variants, a procedure for affinity purification using the target ligand, and a means of evaluating the results of binding enrichments (see for example, U.S. Pat. Nos. 5,223,409, 5,403,484, 5,571,689, and 5,663,143).

Most phage display methods use filamentous phage (such as M13 phage). Lambdoid phage display systems (see WO1995/34683, U.S. Pat. No. 5,627,024), T4 phage display systems (Ren et al. (1998) Gene 215:439; Zhu et al. (1998) Cancer Research, 58:3209-3214; Jiang et al., (1997) Infection & Immunity, 65:4770-4777; Ren et al. (1997) Gene, 195:303-311; Ren (1996) Protein Sci., 5:1833; Efimov et al. (1995) Virus Genes, 10:173) and T7 phage display systems (Smith and Scott (1993) Methods in Enzymology, 217: 228-257; U.S. Pat. No. 5,766,905) are also known.

Many other improvements and variations of the basic phage display concept have now been developed. These improvements enhance the ability of display systems to screen peptide libraries for binding to selected target molecules and to display functional proteins with the potential of screening these proteins for desired properties. Combinatorial reaction devices for phage display reactions have been developed (WO 1998/14277) and phage display libraries have been used to analyze and control bimolecular interactions (WO 1998/20169; WO 1998/20159) and properties of constrained helical peptides (WO 1998/20036). WO 1997/35196 describes a method of isolating an affinity ligand in which a phage display library is contacted with one solution in which the ligand will bind to a target molecule and a second solution in which the affinity ligand will not bind to the target molecule, to selectively isolate binding ligands. WO 1997/46251 describes a method of biopanning a random phage display library with an affinity purified antibody and then isolating binding phage, followed by a micropanning process using microplate wells to isolate high affinity binding phage. Such method can be applied to the libraries of antigen binding domain variants disclosed herein. The use of Staphylococcus aureus protein A as an affinity tag has also been reported (Li et al. (1998) Mol Biotech. 9:187). WO 1997/47314 describes the use of substrate subtraction libraries to distinguish enzyme specificities using a combinatorial library which may be a phage display library. Additional methods of selecting specific binding proteins are described in U.S. Pat. Nos. 5,498,538, 5,432,018, and WO 1998/15833. Methods of generating peptide libraries and screening these libraries are also disclosed in U.S. Pat. Nos. 5,723,286, 5,432,018, 5,580,717, 5,427,908, 5,498,530, 5,770,434, 5,734,018, 5,698,426, 5,763,192, and 5,723,323.

Exemplary Antigens/Target Molecules

Examples of molecules that may be targeted by an antibody (e.g., bispecific or multispecific antibody) produced using a method provided herein include, but are not limited to, soluble serum proteins and their receptors and other membrane bound proteins (e.g., adhesins). In another embodiment, a multispecific antigen-binding protein provided herein is capable of binding one, two or more cytokines, cytokine-related proteins, and cytokine receptors selected from the group consisting of 8MPI, 8MP2, 8MP38 (GDFIO), 8MP4, 8MP6, 8MP8, CSFI (M-CSF), CSF2 (GM-CSF), CSF3 (G-CSF), EPO, FGF1 (ccFGF), FGF2 (βFGF), FGF3 (int-2), FGF4 (HST), FGF5, FGF6 (HST-2), FGF7 (KGF), FGF9, FGF1 0, FGF11, FGF12, FGF12B, FGF14, FGF16, FGF17, FGF19, FGF20, FGF21, FGF23, IGF1, IGF2, IFNA1, g1.

IFNA2, IFNA4, IFNA5, IFNA6, IFNA7, IFN81, IFNG, IFNWI, FEL1, FEL1 (EPSELON), FEL1 (ZETA), IL 1A, IL 1B, IL2, IL3, IL4, ILS, IL6, IL7, IL8, IL9, IL1 0, IL 11, IL 12A, IL 12B, IL 13, IL 14, IL 15, IL 16, IL 17, IL 17B, IL 18, IL 19, IL20, IL22, IL23, IL24, IL25, IL26, IL27, IL28A, IL28B, IL29, IL30, PDGFA, PDGFB, TGFA, TGFB1, TGFB2, TGFBb3, LTA (TNF-β), LTB, TNF (TNF-α), TNFSF4 (OX40 ligand), TNFSF5 (CD40 ligand), TNFSF6 (FasL), TNFSF7 (CD27 ligand), TNFSF8 (CD30 ligand), TNFSF9 (4-1 BB ligand), TNFSF10 (TRAIL), TNFSF11 (TRANCE), TNFSF12 (APO3L), TNFSF13 (April), TNFSF13B, TNFSF14 (HVEM-L), TNFSF15 (VEGI), TNFSF18, HGF (VEGFD), VEGF, VEFGA, VEGFB, VEGFC, IL1R1, IL1R2, IL1RL1, IL1RL2, IL2RA, IL2RB, IL2RG, IL3RA, IL4R, IL5RA, IL6R, IL7R, IL8RA, IL8RB, IL9R, IL10RA, IL10RB, IL 11RA, IL12RB1, IL12RB2, IL13RA1, IL13RA2, IL15RA, IL17R, IL18R1, IL20RA, IL21R, IL22R, IL1HY1, IL1RAP, IL1RAPL1, IL1RAPL2, IL1RN, IL6ST, IL18BP, IL18RAP, IL22RA2, AIF1, HGF, LEP (leptin), PTN, and THPO.

In another embodiment, a target molecule is a chemokine, chemokine receptor, or a chemokine-related protein selected from the group consisting of CCLI (1-309), CCL2 (MCP-1/MCAF), CCL3 (MIP-Iα) CCL4 (MIP-Iβ), CCLS (RANTES), CCL7 (MCP-3), CCL8 (mcp-2), CCL11 (eotaxin), CCL 13 (MCP-4), CCL 15 (MIP-Iδ), CCL 16 (HCC-4), CCL 17 (TARC), CCL 18 (PARC), CCL 19 (MDP-3b), CCL20 (MIP-3α) CCL21 (SLC/exodus-2), CCL22 (MDC/STC-1), CCL23 (MPIF-1), CCL24 (MPIF-2/eotaxin-2), CCL25 (TECK), CCL26 (eotaxin-3), CCL27 (CTACK/ILC), CCL28, CXCLI (GROI), CXCL2 (GR02), CXCL3 (GR03), CXCLS (ENA-78), CXCL6 (GCP-2), CXCL9 (MIG), CXCL 10 (IP 10), CXCL 11 (1-TAC), CXCL 12 (SDFI), CXCL 13, CXCL 14, CXCL 16, PF4 (CXCL4), PPBP (CXCL7), CX3CL 1 (SCYDI), SCYEI, XCLI (lymphotactin), XCL2 (SCM-Iβ), BLRI (MDR15), CCBP2 (D6/JAB61), CCRI (CKRI/HM145), CCR2 (mcp-IRB IRA), CCR3 (CKR3/CMKBR3), CCR4, CCRS (CMKBR5/ChemR13), CCR6 (CMKBR6/CKR-L3/STRL22/DRY6), CCRI (CKR7/EBII), CCR8 (CMKBR8/TER1/CKR- L1), CCR9 (GPR-9-6), CCRL1 (VSHK1), CCRL2 (L-CCR), XCR1 (GPRS/CCXCR1), CMKLR1, CMKOR1 (RDC1), CX3CR1 (V28), CXCR4, GPR2 (CCR10), GPR31, GPR81 (FKSG80), CXCR3 (GPR9/CKR-L2), CXCR6 (TYMSTR/STRL33/Bonzo), HM74, IL8RA (IL8Rcc), IL8RB (IL8Rβ), LTB4R (GPR16), TCP10, CKLFSF2, CKLFSF3, CKLFSF4, CKLFSFS, CKLFSF6, CKLFSF7, CKLFSF8, BDNF, C5R1, CSF3, GRCC10 (C10), EPO, FY (DARC), GDF5, HDF1, HDF1α, DL8, PRL, RGS3, RGS13, SDF2, SLIT2, TLR2, TLR4, TREM1, TREM2, and VHL.

In another embodiment an antibody (e.g., bispecific or multispecific antibody) produced using a method provided herein is capable of binding one or more targets selected from the group consisting of ABCF1; ACVR1; ACVR1B; ACVR2; ACVR2B; ACVRL1; ADORA2A; Aggrecan; AGR2; AICDA; AIF1; AIG1; AKAP1; AKAP2; AMH; AMHR2; ANGPTL; ANGPT2; ANGPTL3; ANGPTL4; ANPEP; APC; APOC1; AR; AZGP1 (zinc-a-glycoprotein); B7.1; B7.2; BAD; BAFF (BLys); BAG1; BAI1; BCL2; BCL6; BDNF; BLNK; BLRI (MDR15); BMP1; BMP2; BMP3B (GDF10); BMP4; BMP6; BMP8; BMPR1A; BMPR1B; BMPR2; BPAG1 (plectin); BRCA1; C19orf10 (IL27w); C3; C4A; C5; C5R1; CANT1; CASP1; CASP4; CAV1; CCBP2 (D6/JAB61); CCL1 (1-309); CCL11 (eotaxin); CCL13 (MCP-4); CCL15 (MIP1δ); CCL16 (HCC-4); CCL17 (TARC); CCL18 (PARC); CCL19 (MIP-3β); CCL2 (MCP-1); MCAF; CCL20 (MIP-3α) CCL21 (MTP-2); SLC; exodus-2; CCL22 (MDC/STC-1); CCL23 (MPIF-1); CCL24 (MPIF-2/eotaxin-2); CCL25 (TECK); CCL26 (eotaxin-3); CCL2? (CTACK/ILC); CCL28; CCL3 (MTP-Iα); CCL4 (MDP-Iβ); CCL5(RANTES); CCL7 (MCP-3); CCL8 (mcp-2); CCNA1; CCNA2; CCND1; CCNE1; CCNE2; CCR1 (CKRI/HM145); CCR2 (mcp-IRβ/RA); CCR3 (CKR/ CMKBR3); CCR4; CCR5 (CMKBR5/ChemR13); CCR6 (CMKBR6/CKR-L3/STRL22/DRY6); CCR7 (CKBR7/EBI1); CCR8 (CMKBR8/TER1/CKR-L1); CCR9 (GPR-9-6); CCRL1 (VSHK1); CCRL2 (L-CCR); CD164; CD19; CD1C; CD20; CD200; CD22; CD24; CD28; CD3; CD37; CD38; CD3E; CD3G; CD3Z; CD4; CD40; CD40L; CD44; CD45RB; CD52; CD69; CD72; CD74; CD79A; CD79B; CDS; CD80; CD81; CD83; CD86; CDH1 (E-cadherin); CDH10; CDH12; CDH13; CDH18; CDH19; CDH2O; CDH5; CDH7; CDH8; CDH9; CDK2; CDK3; CDK4; CDK5; CDK6; CDK7; CDK9; CDKN1A (p21/WAF1/Cip1); CDKN1B (p27/Kip1); CDKN1C; CDKN2A (P16INK4a); CDKN2B; CDKN2C; CDKN3; CEBPB; CER1; CHGA; CHGB; Chitinase; CHST10; CKLFSF2; CKLFSF3; CKLFSF4; CKLFSF5; CKLFSF6; CKLFSF7; CKLFSF8; CLDN3;CLDN7 (claudin-7); CLN3; CLU (clusterin); CMKLR1; CMKOR1 (RDC1); CNR1; COL 18A1; COL1A1; COL4A3; COL6A1; CR2; CRP; CSFI (M-CSF); CSF2 (GM-CSF); CSF3 (GCSF); CTLA4; CTNNB1 (b-catenin); CTSB (cathepsin B); CX3CL1 (SCYDI); CX3CR1 (V28); CXCL1 (GRO1); CXCL10 (IP-10); CXCL11 (I-TAC/IP-9); CXCL12 (SDF1); CXCL13; CXCL14; CXCL16; CXCL2 (GRO2); CXCL3 (GRO3); CXCL5 (ENA-78/LIX); CXCL6 (GCP-2); CXCL9 (MIG); CXCR3 (GPR9/CKR-L2); CXCR4; CXCR6 (TYMSTR/STRL33/Bonzo); CYB5; CYC1; CYSLTR1; DAB2IP; DES; DKFZp451J0118; DNCLI; DPP4; E2F1; ECGF1; EDG1; EFNA1; EFNA3; EFNB2; EGF; EGFR; ELAC2; ENG; ENO1; ENO2; ENO3; EPHB4; EPO; ERBB2 (Her-2); EREG; ERK8; ESR1; ESR2; F3 (TF); FADD; FasL; FASN; FCER1A; FCER2; FCGR3A; FGF; FGF1 (ccFGF); FGF10; FGF11; FGF12; FGF12B; FGF13; FGF14; FGF16; FGF17; FGF18; FGF19; FGF2 (bFGF); FGF20; FGF21; FGF22; FGF23; FGF3 (int-2); FGF4 (HST); FGF5; FGF6 (HST-2); FGF7 (KGF); FGF8; FGF9; FGFR3; FIGF (VEGFD); FEL1 (EPSILON); FIL1 (ZETA); FLJ12584; F1125530; FLRTI (fibronectin); FLT1; FOS; FOSL1 (FRA-1); FY (DARC); GABRP (GABAa); GAGEB1; GAGEC1; GALNAC4S-6ST; GATA3; GDF5; GFI1; GGT1; GM-CSF; GNASI; GNRHI; GPR2 (CCR10); GPR31; GPR44; GPR81 (FKSG80); GRCCIO (C10); GRP; GSN (Gelsolin); GSTP1; HAVCR2; HDAC4; HDAC5; HDAC7A; HDAC9; HGF; HIF1A; HOPI; histamine and histamine receptors; HLA-A; HLA-DRA; HM74; HMOXI ; HUMCYT2A; ICEBERG; ICOSL; 1D2; IFN-a; IFNA1; IFNA2; IFNA4; IFNA5; IFNA6; IFNA7; IFNB1; IFNgamma; DFNW1; IGBP1; IGF1; IGF1R; IGF2; IGFBP2; IGFBP3; IGFBP6; IL-1; IL10; IL10RA; IL10RB; IL11; IL11RA; IL-12; IL12A; IL12B; IL12RB1; IL12RB2; IL13; IL13RA1; IL13RA2; IL14; IL15; IL15RA; IL16; IL17; IL17B; IL17C; IL17R; IL18; IL18BP; IL18R1; IL18RAP; IL19; IL1IA; IL1B; ILIF10; IL1F5; IL1F6; IL1F7; IL1F8; IL1F9; IL1HY1; IL1R1; IL1R2; IL1RAP; IL1RAPL1; IL1RAPL2; IL1RL1; IL1RL2, ILIRN; IL2; IL20; IL20RA; IL21 R; IL22; IL22R; IL22RA2; IL23; IL24; IL25; IL26; IL27; IL28A; IL28B; IL29; IL2RA; IL2RB; IL2RG; IL3; IL30; IL3RA; IL4; IL4R; ILS; IL5RA; IL6; IL6R; IL6ST (glycoprotein 130); EL7; EL7R; EL8; IL8RA; DL8RB; IL8RB; DL9; DL9R; DLK; INHA; INHBA; INSL3; INSL4; IRAK1; ERAK2; ITGA1; ITGA2; ITGA3; ITGA6 (a6 integrin); ITGAV; ITGB3; ITGB4 (b4 integrin); JAG1; JAK1; JAK3; JUN; K6HF; KAI1; KDR; KITLG; KLF5 (GC Box BP); KLF6; KLKIO; KLK12; KLK13; KLK14; KLK15; KLK3; KLK4; KLK5; KLK6; KLK9; KRT1; KRT19 (Keratin 19); KRT2A; KHTHB6 (hair-specific type H keratin); LAMAS; LEP (leptin); Lingo-p75; Lingo-Troy; LPS; LTA (TNF-b); LTB; LTB4R (GPR16); LTB4R2; LTBR; MACMARCKS; MAG or OMgp; MAP2K7 (c-Jun); MDK; MIB1; midkine; MEF; MIP-2; MKI67; (Ki-67); MMP2; MMP9; MS4A1; MSMB; MT3 (metallothionectin-111); MTSS1; MUC1 (mucin); MYC; MY088; NCK2; neurocan; NFKB1; NFKB2; NGFB (NGF); NGFR; NgR-Lingo; NgR- Nogo66 (Nogo); NgR-p75; NgR-Troy; NME1 (NM23A); NOX5; NPPB; NR0B1; NROB2; NR1D1; NR1D2; NR1H2; NR1H3; NR1H4; NR112; NR113; NR2C1; NR2C2; NR2E1; NR2E3; NR2F1; NR2F2; NR2F6; NR3C1; NR3C2; NR4A1; NR4A2; NR4A3; NR5A1; NR5A2; NR6A1; NRP1; NRP2; NT5E; NTN4; ODZI; OPRD1; P2RX7; PAP; PART1; PATE; PAWR; PCA3; PCNA; POGFA; POGFB; PECAM1; PF4 (CXCL4); PGF; PGR; phosphacan; PIAS2; PIK3CG; PLAU (uPA); PLG; PLXDC1; PPBP (CXCL7); PPID; PRI; PRKCQ; PRKDI; PRL; PROC; PROK2; PSAP; PSCA; PTAFR; PTEN; PTGS2 (COX-2); PTN; RAC2 (p21 Rac2); RARB; RGSI; RGS13; RGS3; RNF110 (ZNF144); ROBO2; S100A2; SCGB1D2 (lipophilin B); SCGB2A1 (mammaglobin2); SCGB2A2 (mammaglobin 1); SCYEI (endothelial Monocyte-activating cytokine); SDF2; SERPINA1; SERPINA3; SERP1NB5 (maspin); SERPINE1(PAI-1); SERPDMF1; SHBG; SLA2; SLC2A2; SLC33A1; SLC43A1; SLIT2; SPPI; SPRR1B (Sprl); ST6GAL1; STABI; STAT6; STEAP; STEAP2; TB4R2; TBX21; TCPIO; TOGFI; TEK; TGFA; TGFBI; TGFB1II; TGFB2; TGFB3; TGFBI; TGFBRI; TGFBR2; TGFBR3; THIL; THBSI (thrombospondin-1); THBS2; THBS4; THPO; TIE (Tie-1); TMP3; tissue factor; TLR1; TLR2; TLR3; TLR4; TLR5; TLR6; TLR7; TLR8; TLR9; TLR10; TNF; TNF-a; TNFAEP2 (B94); TNFAIP3; TNFRSFIIA; TNFRSF1A; TNFRSF1B; TNFRSF21; TNFRSF5; TNFRSF6 (Fas); TNFRSF7; TNFRSF8; TNFRSF9; TNFSF10 (TRAIL); TNFSF11 (TRANCE); TNFSF12 (APO3L); TNFSF13 (April); TNFSF13B; TNFSF14 (HVEM-L); TNFSF15 (VEGI); TNFSF18; TNFSF4 (OX40 ligand); TNFSF5 (CD40 ligand); TNFSF6 (FasL); TNFSF7 (CD27 ligand); TNFSFS (CD30 ligand); TNFSF9 (4-1 BB ligand); TOLLIP; Toll-like receptors; TOP2A (topoisomerase Ea); TP53; TPM1; TPM2; TRADD; TRAF1; TRAF2; TRAF3; TRAF4; TRAF5; TRAF6; TREM1; TREM2; TRPC6; TSLP; TWEAK; VEGF; VEGFB; VEGFC; versican; VHL C5; VLA-4; XCL1 (lymphotactin); XCL2 (SCM-1b); XCRI(GPR5I CCXCRI); YY1; and ZFPM2.

Preferred molecular target molecules for antibodies (e.g., bispecific or multispecific antibodies) produced using a method provided herein include CD proteins such as CD3, CD4, CDS, CD16, CD19, CD20, CD34; CD64, CD200 members of the ErbB receptor family such as the EGF receptor, HER2, HER3 or HER4 receptor; cell adhesion molecules such as LFA-1, Mac1, p150.95, VLA-4, ICAM-1, VCAM, alpha4/beta7 integrin, and alphav/beta3 integrin including either alpha or beta subunits thereof (e.g., anti-CD11a, anti-CD18, or anti-CD11b antibodies); growth factors such as VEGF-A, VEGF-C; tissue factor (TF); alpha interferon (alphaIFN); TNFalpha, an interleukin, such as IL-1 beta, IL-3, IL-4, IL-5, IL-S, IL-9, IL-13, IL 17 AF, IL-1S, IL-13R alpha1, IL13R alpha2, IL-4R, IL-5R, IL-9R, IgE; blood group antigens; flk2/flt3 receptor; obesity (OB) receptor; mpl receptor; CTLA-4; RANKL, RANK, RSV F protein, protein C etc.

In one embodiment, an antibody (e.g., bispecific or multispecific antibody) produced using a method provided herein binds low density lipoprotein receptor-related protein (LRP)-1 or LRP-8 or transferrin receptor, and at least one target selected from the group consisting of 1) beta-secretase (BACE1 or BACE2), 2) alpha-secretase, 3) gamma-secretase, 4) tau-secretase, 5) amyloid precursor protein (APP), 6) death receptor 6 (DR6), 7) amyloid beta peptide, 8) alpha-synuclein, 9) Parkin, 10) Huntingtin, 11) p75 NTR, and 12) caspase-6.

In one embodiment, an antibody (e.g., bispecific or multispecific antibody) produced using a method provided herein binds to at least two target molecules selected from the group consisting of: IL-1 alpha and IL- 1 beta, IL-12 and IL-1S; IL-13 and IL-9; IL-13 and IL-4; IL-13 and IL-5; IL-5 and IL-4; IL-13 and IL-1beta; IL-13 and IL- 25; IL-13 and TARC; IL-13 and MDC; IL-13 and MEF; IL-13 and TGF--; IL-13 and LHR agonist; IL-12 and TWEAK, IL-13 and CL25; IL-13 and SPRR2a; IL-13 and SPRR2b; IL-13 and ADAMS, IL-13 and PED2, IL17A and IL 17F, CD3 and CD19, CD138 and CD20; CD138 and CD40; CD19 and CD20; CD20 and CD3; CD3S and CD13S; CD3S and CD20; CD3S and CD40; CD40 and CD20; CD-S and IL-6; CD20 and BR3, TNF alpha and TGF-beta, TNF alpha and IL-1 beta; TNF alpha and IL-2, TNF alpha and IL-3, TNF alpha and IL-4, TNF alpha and IL-5, TNF alpha and IL6, TNF alpha and IL8, TNF alpha and IL-9, TNF alpha and IL-10, TNF alpha and IL-11, TNF alpha and IL-12, TNF alpha and IL-13, TNF alpha and IL-14, TNF alpha and IL-15, TNF alpha and IL-16, TNF alpha and IL-17, TNF alpha and IL-18, TNF alpha and IL-19, TNF alpha and IL-20, TNF alpha and IL-23, TNF alpha and IFN alpha, TNF alpha and CD4, TNF alpha and VEGF, TNF alpha and MIF, TNF alpha and ICAM-1, TNF alpha and PGE4, TNF alpha and PEG2, TNF alpha and RANK ligand, TNF alpha and Te38, TNF alpha and BAFF,TNF alpha and CD22, TNF alpha and CTLA-4, TNF alpha and GP130, TNF a and IL-12p40, VEGF and HER2, VEGF-A and HER2, VEGF-A and PDGF, HER1 and HER2, VEGFA and ANG2,VEGF-A and VEGF-C, VEGF-C and VEGF-D, HER2 and DR5,VEGF and IL-8, VEGF and MET, VEGFR and MET receptor, EGFR and MET, VEGFR and EGFR, HER2 and CD64, HER2 and CD3, HER2 and CD16, HER2 and HER3; EGFR (HER1) and HER2, EGFR and HER3, EGFR and HER4, IL-14 and IL-13, IL-13 and CD40L, IL4 and CD40L, TNFR1 and IL-1 R, TNFR1 and IL-6R and TNFR1 and IL-18R, EpCAM and CD3, MAPG and CD28, EGFR and CD64, CSPGs and RGM A; CTLA-4 and BTN02; IGF1 and IGF2; IGF1/2 and Erb2B; MAG and RGM A; NgR and RGM A; NogoA and RGM A; OMGp and RGM A; POL-1 and CTLA-4; and RGM A and RGM B.

Soluble antigens or fragments thereof, optionally conjugated to other molecules, can be used as immunogens for generating antibodies. For transmembrane molecules, such as receptors, fragments of these (e.g., the extracellular domain of a receptor) can be used as the immunogen. Alternatively, cells expressing the transmembrane molecule can be used as the immunogen. Such cells can be derived from a natural source (e.g., cancer cell lines) or may be cells which have been transformed by recombinant techniques to express the transmembrane molecule. Other antigens and forms thereof useful for preparing antibodies will be apparent to those in the art.

### Activity Assays

An antibody (e.g., bispecific or multispecific antibody) produced using a method provided herein can be characterized for its physical/chemical properties and biological functions by various assays known in the art. Such assays include, but are not limited to, N-terminal sequencing, amino acid analysis, non-denaturing size exclusion high pressure liquid chromatography (HPLC), mass spectrometry, ion exchange chromatography and papain digestion.

In certain embodiments, the antibody (e.g., bispecific or multispecific antibody) produced using a method provided herein is analyzed for its biological activity. In some embodiments, the antibody (e.g., bispecific or multispecific antibody) produced using a method provided herein is tested for its antigen-binding activity. Antigen-binding assays that are known in the art and can be used herein include, without limitation, any direct or competitive binding assays using techniques such as western blots, radioimmunoassays, ELISA (enzyme linked immnosorbent assay), “sandwich” immunoassays, immunoprecipitation assays, fluorescent immunoassays, and protein A immunoassays.

The foregoing written description is considered to be sufficient to enable one skilled in the art to practice the invention. The following Examples are offered for illustrative purposes only, and are not intended to limit the scope of the present invention in any way. Indeed, various modifications in addition to those shown and described herein will become apparent to those skilled in the art from the foregoing description and fall within the scope of the appended claims.

### EXAMPLES

### Example 1

### Methods and Materials

Antibody Construct Design and Synthesis

All antibodies in the Examples below are numbered using the Kabat (Kabat et al. “Sequences of Proteins of Immunological Interest.” Bethesda, Md.: NIH, 1991) and EU (Edelman et al. “The covalent structure of an entire gammaG immunoglobulin molecule.” Proc Natl Acad Sci USA 1969; 63:78-85) numbering systems for variable and constant domains, respectively. Antibody constructs were generated by gene synthesis (GENEWIZ®) and wherever applicable, sub-cloned into the expression plasmid (pRK5) as described previously (Dillon et al. “Efficient production of bispecific IgG of different isotypes and species of origin in single mammalian cells.”MAbs 2017; 9:213-30). All antibody HC in this study were aglycosylated (N297G mutation) and with the carboxy-terminal lysine deleted (ΔK447) to reduce product heterogeneity and thereby facilitate accurate quantification of BsIgG by LCMS (Dillon et al., infra; Yin et al. “Precise quantification of mixtures of bispecific IgG produced in single host cells by liquid chromatography-Orbitrap high-resolution mass spectrometry.” Mabs 2016; 8:1467-76). The two component HC of all BsIgG in this study were engineered to contain either a ‘knob’ mutation (e.g., T366W) in the first listed antibody or ‘hole’ mutations (e.g., T366S:L368A:Y407V) in the second listed antibody to facilitate HC heterodimerization (Atwell et al. “Stable heterodimers from remodeling the domain interface of a homodimer using a phage display library. J Mol Biol 1997; 270:26-35).

For a few of the BsIgG in this study, FR mutations were judiciously made to provide sufficient mass difference between correctly paired and mispaired BsIgG species for more accurate quantitation by LCMS analysis. The mass difference needed for accurate quantification of bispecific IgG yield is ≤118 Da (Yin et al., infra). Specifically, the antibodies and mutations were anti-HER2 V_{L} R66G when combined with anti-CD3 or variants (in Table A), anti-IL-1β or anti-GFRα (Table B); anti-VEGFA V_{L} F83A when combined with anti-ANG2 or variants (in Table F); anti-CD3 V_{L} N34A:F83A when combined with anti-Factor D 25D7 v1 or anti-IL-33 or anti-HER2 (in Table G2); anti-RSPO3 V_{L} F83A, when combined with anti-CD3; anti-EGFR V_{L} F83A when combined with anti-SIRPα or anti-Factor D 20D12 v1; plus anti-IL-4 V_{L} N31A:F83A when combined with anti-GFRα1 (Table B or FIGS. IA-1F). The chosen residues had no detectable impact on BsIgG yield based upon comparison with parental antibodies.

Antibody Expression and Purification

All BsIgG were transiently expressed in HEK293-derived EXPI293F™ cells as described previously (Dillon et al., supra). Four plasmids corresponding to the two LC and two HC were co-transfected into EXPI293F™ cells (Thermo Fisher Scientific). The LC DNA was varied for each experiment and the highest bispecific yield with the optimal HC:LC ratio was reported as described previously (Dillon et al., supra). The ratio of the two HC was fixed at 1:1. The transfected cell culture (30 mL) was grown for 7 days at 37 ° C. with shaking. BsIgG from the filtered cell culture supernatants were purified in a high throughput fashion by Protein A affinity chromatography (TOYOPEARL® AF-rProtein A, Tosoh Bioscience). Impurities such as aggregates and half IgG₁ were removed by size exclusion chromatography using a ZENIX®-C SEC-300 column (10 mm×300 mm, 3 μm particle size, Sepax Technology). The IgG₁ concentration was calculated using an extinction coefficient A^{0.1%}₂₈₀ₙₘ f 1.5. Purification yield was estimated after protein A chromatography by multiplying the protein concentration with elution volume.

Analytical Characterization of BsIgG by SEC HPLC

BsIgG samples (20 μL) were chromatographed under isocratic conditions via size exclusion chromatography on a TSKGEL® SuperSW3000 column (4.6×150 mm, 4 μm) (Tosoh Bioscience) connected to an HPLC column (DIONEX™ UltiMate 3000, Thermo Fisher Scientific). The mobile phase was 200 mM potassium phosphate and 250 mM potassium chloride at pH 7.2 with a flow rate of 0.3 mL/min with absorbance measurement at a wavelength of 280 nm.

BsIgG Yield Determination by High Resolution LCMS

Quantification of BsIgG yield (intensity of correctly paired LC species over all three mispaired IgG₁ species) was performed via mass spectrometry (Thermo Fisher EXACTIVE™ Plus Extended Mass Range ORBITRAP™) as described previously, and assumes no response bias amongst the different mass peaks (see Yin et al., infra).

For denaturing mass spectrometry, samples (3 μg) were injected onto a reversed-phase liquid chromatography column (MABPAC™, Thermo Fisher Scientific, 2.1 mm×50 mm) heated to 80° C. using a Dionex ULTIMATE™ 3000 rapid separation liquid chromatography (RSLC) system. A binary gradient pump was used to deliver solvent A (99.88% water containing 0.1% formic acid and 0.02% trifluoroacetic acid) and solvent B (90% acetonitrile containing 9.88% water plus 0.1% formic acid and 0.02% trifluoroacetic acid) as a gradient of 20% to 65% solvent B over 4.5 min at 300 μL/min. The solvent was step-changed to 90% solvent B over 0.1 min and held at 90% for 6.4 min to clean the column. Finally, the solvent was step-changed to 20% solvent B over 0.1 min and held for 3.9 min for re-equilibration. Samples were analyzed online via electrospray ionization into the mass spectrometer using the following parameters for data acquisition: 3.90 kV spray voltage; 325° C. capillary temperature; 200 S-lens RF level; 15 sheath gas flow rate and 4 AUX gas flow rate in ESI source; 1,500 to 6,000 m/z scan range; desolvation, in-source CID 100 eV, CE 0; resolution of 17,500 at m/z 200; positive polarity; 10 microscans; 3E6 AGC target; fixed AGC mode; 0 averaging; 25 V source DC offset; 8 V injection flatapole DC; 7 V inter flatapole lens; 6 V bent flatapole DC; 0 V transfer multipole DC tune offset; 0 V C-trap entrance lens tune offset; and trapping gas pressure setting of 2.

For native mass spectrometry, samples (10 μg) were injected onto an Acquity UPLC™ BEH size exclusion chromatography column (Waters, 4.6 mm×150 mm) heated to 30° C. using a Dionex ULTIMATE™ 3000 RSLC system. Isocratic chromatography runs (10 min) utilized an aqueous mobile phase containing 50 mM ammonium acetate at pH 7.0 with a flow rate of 300 μL/min.

Samples were analyzed online via electrospray ionization into the mass spectrometer using the following parameters for data acquisition: 4.0 kV spray voltage; 320° C. capillary temperature; 200 S-lens RF level; 4 sheath gas flow rate and 0 AUX gas flow rate in ESI source; 300 to 20,000 m/z scan range; desolvation, in-source CID 100 eV, CE 0; resolution of 17,500 at m/z 200; positive polarity; 10 microscans; 1E6 AGC target; fixed AGC mode; 0 averaging; 25 V source DC offset; 8 V injection flatapole DC; 7 V inter flatapole lens; 6 V bent flatapole DC; 0 V transfer multipole DC tune offset; 0 V C-trap entrance lens tune offset; and trapping gas pressure setting of 2.

Acquired mass spectral data were analyzed using Protein Metrics Intact Mass™ software and Thermo Fisher BIOPHARMA FINDER™ 3.0 software. The signal intensity of the correctly paired LC species from the deconvolved spectrum of each sample was used for quantification relative to the three mispaired IgG₁ species. HC homodimers and half IgG were either undetectable or present in trace amounts and excluded from the calculations. The correctly LC paired BsIgG were estimated from the isobaric mixture of BsIgG and the double LC mispaired IgG₁ by using the algebraic formula described previously (see Yin et al., infra).

SDS-PAGE gel analysis of BsIgG

BsIgG purified by protein A and size exclusion chromatography were analyzed by SDS-PAGE. The samples were prepared in the presence and absence of DTT for analyzing the electrophoretic mobility in both reducing and non-reducing conditions, respectively. The samples mixed with sample dye were heated at 95 ° C. for 5 min with DTT or for 1 min without DTT and electrophoresed on 4-20% Tris-glycine gels (Bio-Rad) at 120 V. The gels were then stained with GELCODE™ blue protein stain (Thermo Fisher Scientific) and destained in water. Equal amount of protein (6 μg) was loaded for each sample.

Kinetic Binding Experiments

Kinetic binding experiments were performed using surface plasmon resonance on a BIAcore T200 instrument (GE Healthcare). Anti-Fab (GE Healthcare) was immobilized [˜12000 resonance units (RU)] on a CMS sensor chip. Parent and mutant Fabs were captured onto the immobilized surface and the binding of analytes were assessed. Sensorgrams with analyte concentrations of 0, 0.293, 1.17, 4.6875, 18.75, 75, 300 nM for HER2-ECD (in house) and VEGF-C (Cys156Ser) (R&D Systems, catalog number 752-VC); 0, 0.0195, 0.0781, 0.3125, 1.25, 5, 20 mM VEGF165 (R&D Systems, catalog number 293-VE) and IL-13 (in-house); 0, 0.0732, 0.293, 1.17, 4.6875, 18.75, 75 nM MET-R Fc (R&D Systems, catalog number 8614-MT), IL-1β (R&D Systems, catalog number 201-LB/CF), EGFR Fc (R&D Systems, catalog number 344-ER); 0, 0.976, 3.906, 15.625, 62.5, 250 nM biotinylated CD3 (in-house) were generated using an injection time of 3 minutes, a flow rate of 50 μl/min at a temperature of 25° C. The dissociation was monitored for 900 seconds after injection of analyte. The running buffer used was 10 mM HEPES, pH 7.4, 150 mM NaCl, 0.003% EDTA, 0.05% Tween (HBS-EP+, GE Healthcare). The chip surface was regenerated after each injection with 10 mM Glycine, pH 2.1. The sensorgrams were corrected using a double blank referencing (substation of zero-analyte concentration and the blank reference cell). Sensorgrams were then analyzed using a 1:1 Langmuir model by software provided by the manufacturer.

### Example 2

### Elucidating Heavy Chain/Light Chain Pairing Preferences to Facilitate the Assembly of Bispecific IgG in Single Cell

Introduction

In the study described here, high throughput production and high resolution LCMS analysis (Dillon et al. “Efficient production of bispecific IgG of different isotypes and species of origin in single mammalian cells.” MAbs 2017; 9:213-30; Yin et al. Precise quantification of mixtures of bispecific IgG produced in single host cells by liquid chromatography-Orbitrap high-resolution mass spectrometry.” MAbs 2016; 8:1467-76) were utilized to survey 99 different antibody pairs with knob-in-hole HC but without Fab mutations for the yield of BsIgG. One third of antibody pairs showed high (>65%) BsIgG yield, consistent with a strong inherent cognate HC/LC chain pairing preference. Installation of previously identified charge mutations at the two C_{H}1/C_{L} domain interfaces (Dillon et al. “Efficient production of bispecific IgG of different isotypes and species of origin in single mammalian cells.”MAbs 2017; 9:213-30) for such antibody pairs was used to enhance the production of BsIgG. Next, we investigated whether a cognate chain pairing preference in one or both arms was needed for high yield of BsIgG. Mutational analysis was used to identify specific residues in CDR H3 and L3 contributing to high BsIgG yield. The CDR H3 and L3 and specific residues identified were then inserted into other available, unrelated antibodies that show random HC/LC chain pairing to determine their effect upon BsIgG yield. Finally, mutational analysis was used to investigate the effect of the interchain disulfide bond upon yield of BsIgG.

Influence of Constituent Antibody Pairs on the Yield of BsIgG

Previously, high yields of BsIgG (>65%) with knob-in-hole heavy chain (HC) mutations but without Fab arm mutations were observed for two bispecifics, namely, anti-EGFR/MET and anti-IL-13/IL-4 (Dillon et al., infra). To investigate the strength and frequency of occurrence of cognate heavy chain/light chain (HC/LC) pairing preference, a large panel of antibody pairs (n =99) was used to generate BslgGs. For simplicity, all bispecifics in this study were constructed with human IgG₁ HC constant domains. Six antibodies binding to either IL-13, IL-4, MET, EGFR, HER2 or CD3 (Dillon et al., infra) were used to construct a matrix of all 15 possible BsIgG₁. Next, these six antibodies were permuted with 14 additional antibodies that were mainly κ LC isotype with three λLC isotype (anti-DRS, anti-α₅β₁, anti-RSPO2) (see Table A below;. In Table A, germline gene families were identified by comparing the LC and HC sequences with the human antibody germline gene repertoire using proprietary alignment tool. The closest match with the germline gene segment was reported. All antibodies used in this study were humanized antibodies except the three fully human antibodies (anti-CD33, anti-PDGF-C, anti-Flu B).

TABLE A
Germline gene family and LC isotype analysis of different antibodies
that were evaluated for LC/HC pairing preferences.

| | Antigen- | | Germline | | |
|---|---|---|---|---|---|
| Antibody/ | binding | LC | gene family | | |
| Clone | specificity | isotype | V_{L} | V_{H} | Ref. |
| Lebrikizumab | IL-13 | κ | KV4 | HV2 | Ultsch et al. |
| 19C11 | IL-4 | κ | KV1 | HV3 | Spiess et al. |
| Onartuzumab/ | MET | κ | KV1 | HV3 | Merchant |
| 5D5 | | | | | et al. |
| D1.5 | EGFR | κ | KV1 | HV3 | Schaefer et al. |
| Trastuzumab/ | HER2 | κ | KV1 | HV3 | Carter et al. |
| humAb4D5-8 | | | | | |
| humAbUCHT | CD3 | κ | KV1 | HV3 | Rodrigues |
| 1 v9 | | | | | et al. |
| 25D7 v1 | Factor D | κ | KV4 | HV2 | na |
| 5D6 | RSPO3 | κ | KV1 | HV4 | na |
| 10C12 | IL-33 | κ | KV3 | HV3 | na |
| 19D1 v4.1 | SIRPα | κ | KV1 | HV1 | na |
| 20D12 v1 | Factor D | κ | KV1 | HV1 | na |
| 8E11 v2 | LGR5 | κ | KV4 | HV1 | na |
| 2H12 v6.11 | IL-1β | κ | KV1 | HV3 | na |
| 7C9 v8 | GFRαl | κ | KV1 | HV3 | Bhakta et al. |
| Apomab | DR5 | λ | LV3 | HV3 | Adams et al. |
| 1A1 | RSPO2 | λ | LV2 | HV3 | na |
| na | α₅β₁ | λ | LV3 | HV3 | na |
| 46B8 | FluB | κ | KV2 | HV5 | na |
| 1E5 v3.1 | PDGF-C | κ | KV4 | HV1 | na |
| GM15.33 | CD33 | κ | KV2 | HV1 | na |

KV = κ variable; LV = λ variable, HV = heavy variable; na = not available.
1. Merchant M, Ma X, Maun HR, Zheng Z, Peng J, Romero M, Huang A, Yang NY, Nishimura M, Greve J, et al. Monovalent antibody design and mechanism of action of onartuzumab, a MET antagonist with anti-tumor activity as a therapeutic agent. Proc Natl Acad Sci U.S.A. 2013; 110: E2987-96.
2. Schaefer G, Haber L, Crocker LM, Shia S, Shao L, Dowbenko D, Totpal K, Wong A, Lee CV, Stawicki S, et al. A two-in-one antibody against HER3 and EGFR has superior inhibitory activity compared with monospecific antibodies. Cancer Cell 2011; 20: 472-86.
5. Ultsch M, Bevers J, Nakamura G, Vandlen R, Kelley RF, Wu LC, Eigenbrot C. Structural basis of signaling blockade by anti-IL-13 antibody lebrikizumab. J Mol Biol 2013; 425: 1330-9.
6. Spiess C, Bevers J, 3rd, Jackman J, Chiang N, Nakamura G, Dillon M, Liu H, Molina P, Elliott JM, Shatz W, et al. Development of a human IgG4 bispecific antibody for dual targeting of interleukin-4 (IL-4) and interleukin-13 (IL-13) cytokines. J Biol Chem 2013; 288: 26583-93.
8. Carter P, Presta L, Gorman CM, Ridgway JB, Henner D, Wong WL, Rowland AM, Kotts C, Carver ME, Shepard HM. Humanization of an anti-p185HER2 antibody for human cancer therapy. Proc Natl Acad Sci U.S.A. 1992; 89: 4285-9.
9. Rodrigues ML, Shalaby MR, Werther W, Presta L, Carter P. Engineering a humanized bispecific F(ab′)2 fragment for improved binding to T cells. Int J Cancer Suppl 1992; 7: 45-50.
10. Bhakta S, Crocker LM, Chen Y, Hazen M, Schutten MM, Li D, Kuijl C, Ohri R, Zhong F, Poon KA, et al. An anti-GDNF family receptor alpha 1 (GFRA1) antibody-drug conjugate for the treatment of hormone receptor-positive breast cancer. Mol Cancer Ther 2018; 17: 638-49.
11. Adams C, Totpal K, Lawrence D, Marsters S, Pitti R, Yee S, Ross S, Deforge L, Koeppen H, Sagolla M, et al. Structural and functional analysis of the interaction between the agonistic monoclonal antibody Apomab and the proapoptotic receptor DRS. Cell Death Differ 2008; 15: 751-61.

Next, antibody pairs shown in Table B below were co-expressed in HEK293-derived EXPI293FTM cells at optimized chain ratios, and the yield of BsIgG was determined with an improved version of a previously described method (see Dillon et al., Yin et al., infra). None of the antibody pairs contained Fab mutations described in Dillon et al. (infra). All bispecific antibody pairs comprised knob-in-hole mutations for heavy chain heterodimerization.

Following co-expression of antibody pairs and protein A chromatography, the purified IgG₁ pools were further purified by size exclusion chromatography (SEC) to remove any small quantities of aggregates and half IgG₁ present prior to quantitation by high resolution LCMS. The yield of correctly assembled BsIgG in isobaric (i.e., same molecular mass) mixtures that also contained LC-scrambled IgG₁ was estimated using a previously developed algebraic formula (see Yin et al., infra). Data shown in Table B are the yield of BsIgG from optimized LC DNA ratios. BsIgG yields >65% are indicated in bold. The HC of mAb-1 contained the ‘hole’ mutations (T366S:S368A:Y407V) and the HC for mAb-2 contained a ‘knob’ mutation (T366W) (Atwell et al. “Stable heterodimers from remodeling the domain interface of a homodimer using a phage display library.” J Mol Biol 1997; 270:26-35).

TABLE B
Half Antibody pairs used to investigate BsIgG yield

| | mAb-1 | | | | | |
|---|---|---|---|---|---|---|
| mAb-2 | IL-13 | MET | EGFR | CD3 | IL-4 | HER2 |
| IL-13 | NA | 87.6 | 87.0 | 75.2 | 70.3 | 66.6 |
| MET | 86.6 | NA | 72.3 | 60.7 | 53.1 | 59.9 |
| EGFR | 86.3 | 72.4 | NA | 23.9 | 45.4 | 22.0 |
| CD3 | 75.5 | 54.8 | 32.5 | NA | 25.0 | 22.7 |
| IL-4 | 68.7 | 58.0 | 44.1 | 26.9 | NA | 22.6 |
| HER2 | 64.6 | 65.4 | 21.6 | 24.1 | 25.0 | NA |
| DR5 | 90.4 | 95.1 | 53.3 | 53.4 | 53.8 | 34.7 |
| FluB | 87.7 | 69.5 | 52.3 | 32.0 | 60.8 | 72.7 |
| RSPO3 | 84.7 | 58.6 | 82.1 | 40.6 | 26.0 | 22.0 |
| Factor D 25D7 v1 | 83.6 | 73.1 | 69.3 | 83.1 | 35.5 | 68.7 |
| RSPO2 | 83.5 | 51.1 | 78.5 | 38.7 | 22.3 | 71.3 |
| IL-13 | 74.2 | 63.5 | 80.4 | 77.8 | 63.7 | 65.9 |
| GFRα1 | 73.9 | 40.6 | 77.5 | 79.6 | 33.5 | 68.0 |
| PDGF-C | 61.2 | 71.0 | 54.6 | 56.0 | 34.2 | 24.3 |
| CD33 | 49.8 | 58.8 | 49.6 | 36.4 | 56.5 | 51.5 |
| α₅β₁ | 45.9 | 62.2 | 31.0 | 41.4 | 48.4 | 72.6 |
| IL-33 | 45.6 | 21.4 | 30.9 | 20.4 | 42.4 | 46.6 |
| SIRPα | 41.7 | 31.0 | 22.6 | 60.6 | 47.9 | 31.8 |
| Factor D 20D12 v1 | 23.5 | 29.8 | 58.0 | 36.0 | 22.6 | 69.6 |
| LGR5 | 21.7 | 56.2 | 53.8 | 23.6 | 22.8 | 22.1 |

NA = not applicable; monospecific antibodies.

The yield of BsIgG₁ for the 99 unique antibody pairs varied over a very wide range: 22-95% (see Table B). Strikingly, non-random HC/LC pairing (>30% yield of BsIgG₁) was observed for the majority (>80%) of antibody pairs with high (>65%) and intermediate (30-65%) yield of BsIgG₁ seen for 33 and 48 antibody pairs, respectively. Near quantitative (>90%) formation of BsIgG₁ was measured for two antibody pairs (anti-MET/DR5 and anti-IL-13/DR5).

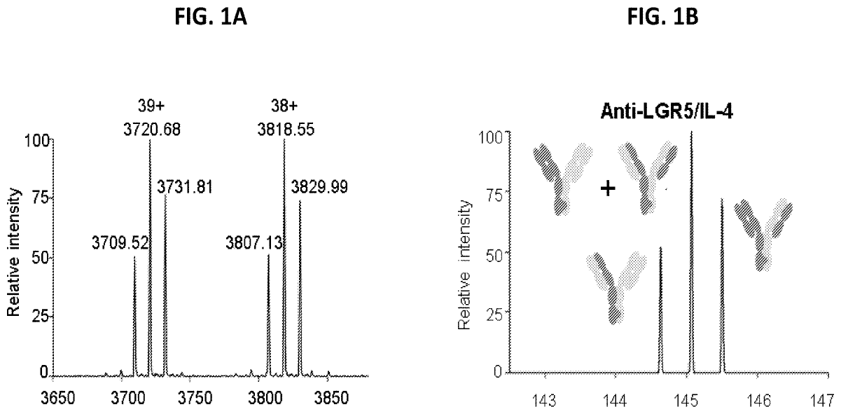

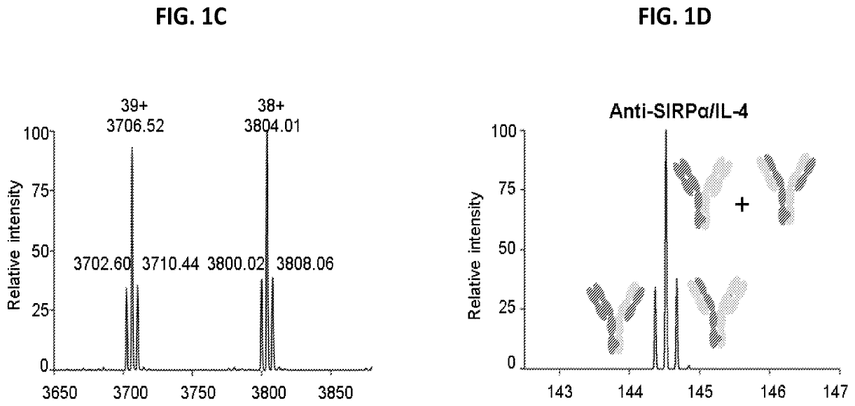

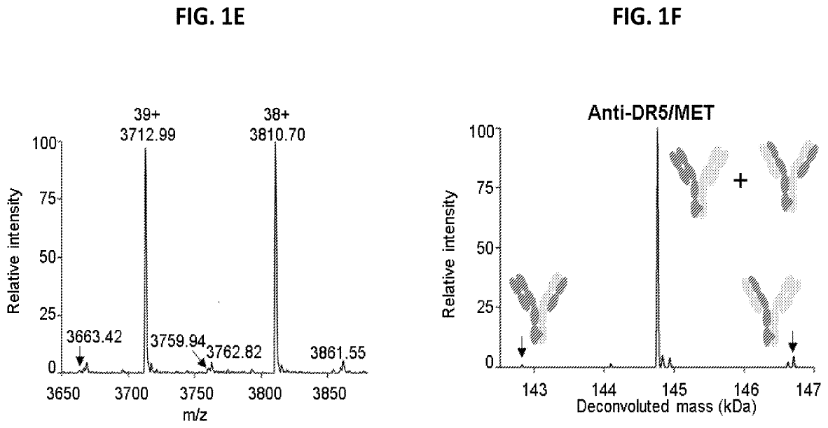

FIGS. 1A-1F show high resolution LCMS data for representative examples of low yield (<30%, e.g., anti-LGRS/IL-4, see FIGS. 1A and 1B) intermediate yield (30%-65%, e.g., anti-SIRPα/IL-4, see FIGS. 1C and 1D) and high yield (>65%, e.g., anti-MET/DR5, see FIGS. 1E and 1F) of BsIgG₁. Corresponding antibody pairs were transiently co-transfected into HEK293-derived EXPI293F™ cells. The IgG₁ species were purified by protein A chromatography and size exclusion chromatography before quantification of the BsIgG₁ yield by high resolution LCMS, as described in Dillon et al., infra and Yin et al., infra. Data shown in FIGS. 1A, 1C, and 1E are mass envelopes for charge states 38+ and 39+, and FIGS. 1B, 1D, and 1F show corresponding deconvoluted data and provide cartoons representing the different IgG₁ species present.

The BsIgG₁ yield for each antibody studied varied over a wide range depending upon its partner antibody. For example, the BsIgG₁ yield for the anti-MET antibody varied from as little as ˜21% when paired with anti-IL-33 to as much as ˜95% when paired with anti-DR5 (Table B). To investigate any influence of ‘knob’ and ‘hole’ mutations on the cognate HC/LC pairing preference, BsIgG₁ were produced with the HC containing the ‘knob’ mutation in mAb1 and ‘hole’ mutations in mAb2 or vice versa (Table B). The yield of BsIgG₁ was minimally influenced by which HC contained the ‘knob’ and ‘hole’ mutations in all cases (n=15) tested (Table B). The recovery of IgG species from 30 mL cultures by protein A chromatography varied over ˜5-fold (1.5 to 8.0 mg)

The results above indicated that high yield of BsIgG₁ without Fab mutations is a common phenomenon that depends on the constituent antibody pairs

Effect of C_{H}1/C_{L}Interface Charge Mutations on Yield of BsIgG1 for Antibody Pairs with a Cognate HC/LC Paring Preference

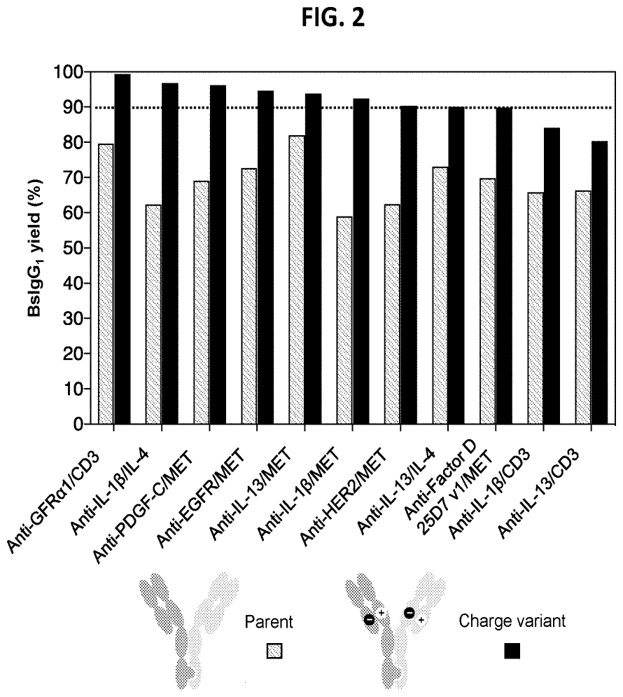

Previously, a combination of mutations at all four domain/domain interfaces (i.e., both V_{H}/V_{L} and both C_{H}1/C_{L}) in conjunction with knob-into-hole HC mutations was used for near quantitative assembly of BsIgG of different isotypes in single mammalian host cells (see Dillon et al., infra). Here, antibody pairs that give high yield of BsIgG₁ without any Fab mutations were identified (Table B). These antibody pairs differ in their variable domain sequences whereas the constant domains, namely IgG₁ C_{H}1 and k C_{L}, were identical in most cases. It was hypothesized that for such antibody pairs, mutations at the two C_{H}1/C_{L} interfaces alone might be sufficient to enhance the yield of correctly assembled bispecific to ˜100%. Eleven different antibody pairs were selected, and the yield of BsIgG₁ compared in the presence or absence of previously reported C_{H}1/C_{L} domain interface charge mutations (see Dillon et al., infra). Specifically, the ‘knob’ arms were engineered with CL V133E and C_{H}1 S183K mutations and the ‘hole’ arm with C_{L} V133K and C_{H}1 S183E mutations (see Dillon et al., infra). The charge mutations at the two C_{H}1/C_{L} interfaces increased the BsIgG₁ yield for all antibody pairs by ˜12-34% to ≥90% BsIgG₁ yield in the majority (9/11) of cases (FIG. 2). For the charge pair variants in FIG. 2, the first listed antibody in the pair contains the C_{L} V133E and C_{H}1 S183K mutations, and the second listed antibody contains the C_{L} V133K and C_{H}1 S183E mutations (see Dillon et al., infra). 90% yield of BsIgG₁ is indicated by the dotted horizontal line in FIG. 2. The the C_{L} V133E and C_{H}1 S183K mutations did not affect the antibodies' affinities for their target antigens (data not shown).

Effect of Cognate HC/LC Pairing Preference in One Arm of a BsIgG on Yield of the BsIgG

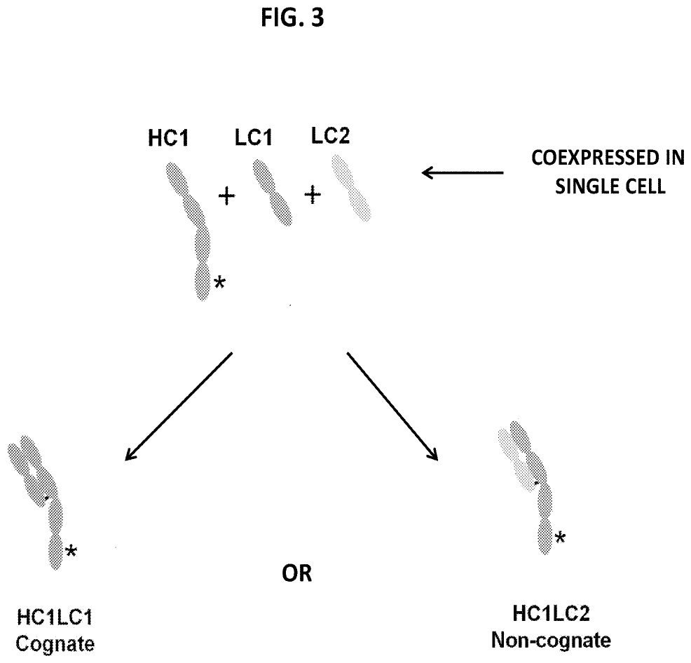

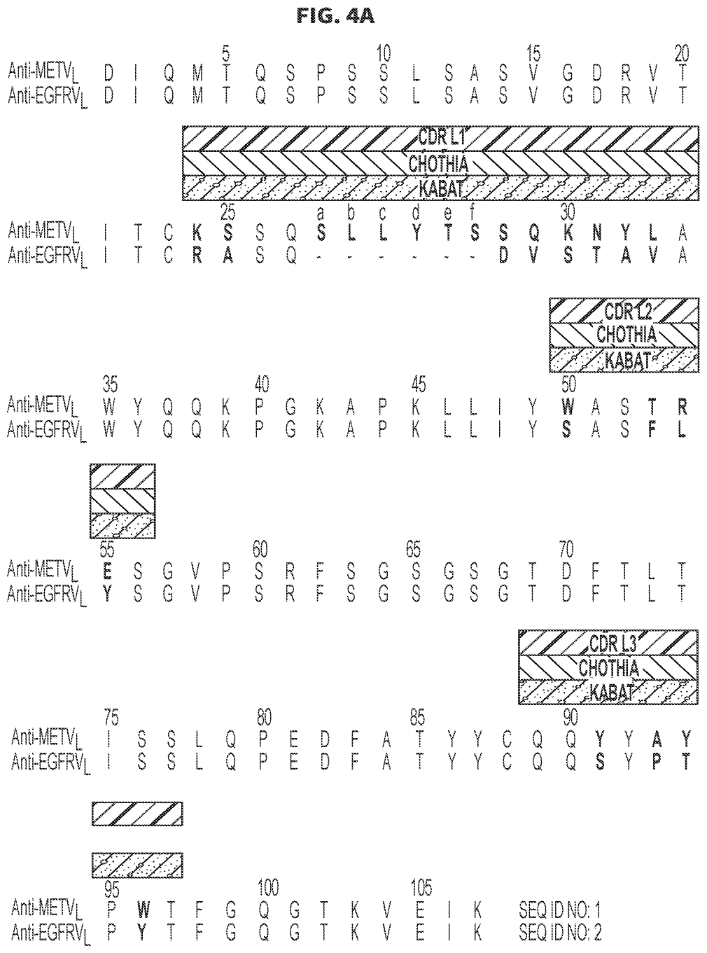

The mechanistic bases for high yields of BsIgG₁ observed for some antibody pairs were investigated. Two antibody pairs, namely anti-EGFR/MET and anti-IL-4/IL-13, were selected for this study based on their high yield of BsIgG₁ without Fab mutations (see Table B and Dillon et al., infra). A priori, either one or both Fab may exhibit a cognate HC/LC pairing preference contributing to the high yield of BsIgG₁. Three chain co-expression experiments were undertaken to distinguish between these possibilities. A single HC (HC1) with either ‘knob’ or ‘hole’ mutations was transiently co-expressed in Expi293F™ cells with its cognate LC (LC1) and a competing non-cognate LC (LC2) (FIG. 3). The asterisks in FIG. 3 denote the presence of either “knob” or “hole” mutations in the HC. (The HC of anti-EGFR, anti-IL13, and anti-HER2 contain a “knob” mutation (T366W), whereas the HC of anti-MET, anti-IL4, and anti-CD3 contain “hole” mutations (T366S : S368A : Y407V) (see Atwell et al. “Stable heterodimers from remodeling the domain interface of a homodimer using a phage display library. J Mol Biol 1997; 270:26-35).) The resultant half IgG species were purified from the corresponding cell culture supernatant by protein A affinity chromatography and the extent of cognate and non-cognate HC/LC pairing assessed by high resolution LCMS (Dillon et al. and Yin et al., infra). The percentage of cognate HC/LC pairing was calculated by quantifying the half IgG₁ species.

As shown in Table C below, the anti-MET HC shows a strong preference for its cognate LC (˜71%) over the non-cognate anti-EGFR LC, whereas the anti-EGFR HC shows only a slight preference for its cognate LC (˜56%) over the non-cognate anti-MET LC. The anti-IL-13 HC shows a strong preference for its cognate LC (81%) over the non-cognate anti-IL-4 LC, whereas the anti-IL-4 HC shows no preference (49%) for its cognate LC. These data are consistent with the notion that the high BsIgG₁ yield for anti-EGFR/MET results from the strong and weak cognate HC/LC pairing preference for the anti-MET and anti-EGFR antibodies, respectively. In contrast, the high BsIgG₁ yield for anti-IL-13/IL-4 apparently reflects a strong cognate HC/LC pairing preference for the anti-IL-13 antibody alone. Thus, a cognate HC/LC pairing preference in one or both arms can apparently be sufficient for high yield of BsIgG₁ in a single cell without the need for Fab mutations.

TABLE C
Quantification of Antibody Cognate Chain Preferences Following
Co-Expression.

| | | | HC/LC pairing (%) | |
|---|---|---|---|---|
| HC1 | LC1 | LC2 | Cognate | Non-cognate |
| MET | MET | EGFR | 70.6 | 29.4 |
| EGFR | MET | EGFR | 56.4 | 43.6 |
| IL-13 | IL-13 | IL-4 | 81.0 | 19.0 |
| IL-4 | IL-13 | IL-4 | 49.1 | 50.9 |
| HER2 | HER2 | CD3 | 51.0 | 49.0 |
| CD3 | HER2 | CD3 | 46.4 | 53.6 |

Anti-HER2/CD3, was selected as a control for this study based on its low yield of BsIgGᵢ (see Table B and Dillon et al., infra). The anti-HER2 HC shows no pairing preference for its cognate LC over the non-cognate anti-CD3 LC. Similarly, the anti-CD3 HC shows no pairing preference for its cognate LC over the non-cognate anti-HER2 LC (see Table C).

HC pairing with its cognate light chain (LC) or a non-cognate LC when co-expressed in a single host cell was also evaluated. Briefly, each HC was co-transfected into HEK293-derived EXPI293FTM cells with either its cognate LC or a non-cognate LC. The IgG1 and half IgG1 species were purified from the cell culture supernatant by protein A chromatography and analyzed by LC-MS. (Labrijn et al. “Efficient generation of stable bispecific IgG1 by controlled Fab-arm exchange.” Proc Natl Acad Sci USA 2013; 110:5145-50; Spiess C et al. “Bispecific antibodies with natural architecture produced by co-culture of bacteria expressing two distinct half-antibodies.” Nat Biotechnol 2013; 31:753-8). The percentage of cognate HC/LC pairing was calculated by quantifying half IgG1 species. Protein expression yield was estimated by multiplying the antibody concentration with the elution volume obtained from high-throughput protein A chromatography step. The HC of anti-EGFR, anti-IL-13 and anti-HER2 contain a ‘knob’ mutation (T366W) whereas the HC of anti-MET, anti-IL-4 and anti-CD3 contain ‘hole’ mutations (T366S:S368A:Y407V) (see Spiess et al. “Alternative molecular formats and therapeutic applications for bispecific antibodies.”Mol Immunol 2015; 67:95-106). In the absence of competition, HC can assemble efficiently with a non-cognate LC as judged by all six different mis-matched HC/LC pairs tested (see Table D below).

TABLE D
HC pairing with its cognate light chain (LC) or a non-cognate LC
when co-expressed in a single host cell

| | | Half IgG₁ | Expression yield |
|---|---|---|---|
| HC | LC | HC-LC pairing (%) | (mg) |
| MET | MET | 100.0 | 6.3 |
| MET | EGFR | 100.0 | 6.7 |
| EGFR | EGFR | 100.0 | 5.1 |
| EGFR | MET | 100.0 | 6.6 |
| IL-13 | IL-13 | 100.0 | 3.0 |
| IL-13 | IL-4 | 100 | 1.9 |
| IL-4 | IL-4 | 100 | 4.8 |
| IL-4 | IL-13 | 100 | 3.1 |
| HER2 | HER2 | 100 | 5.4 |
| HER2 | CD3 | 100 | 6.1 |
| CD3 | CD3 | 100 | 4.1 |
| CD3 | HER2 | 100 | 5.0 |

The Contribution of the Anti MET CDR L3 and CDR H3 to the Yield of Anti-EGFR/MET BsIgG₁

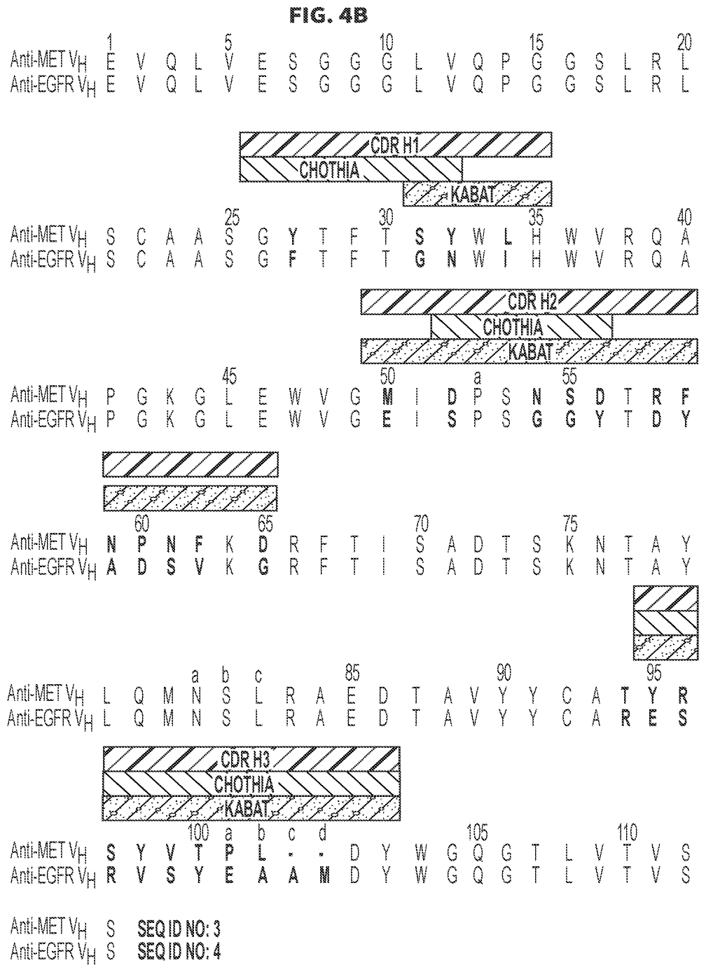

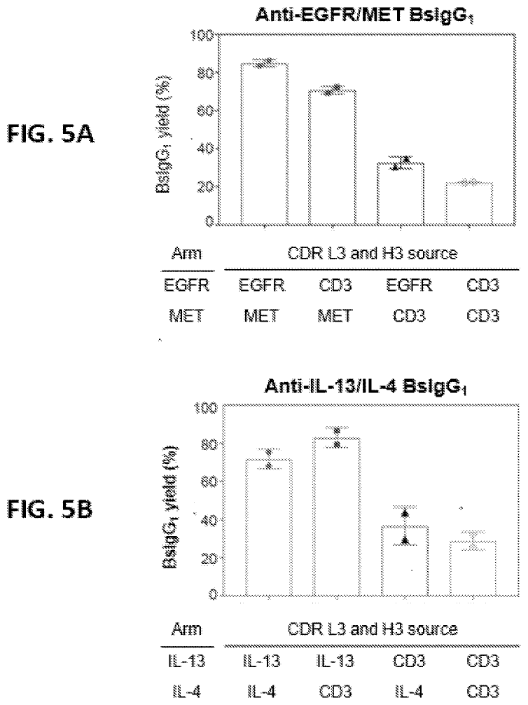

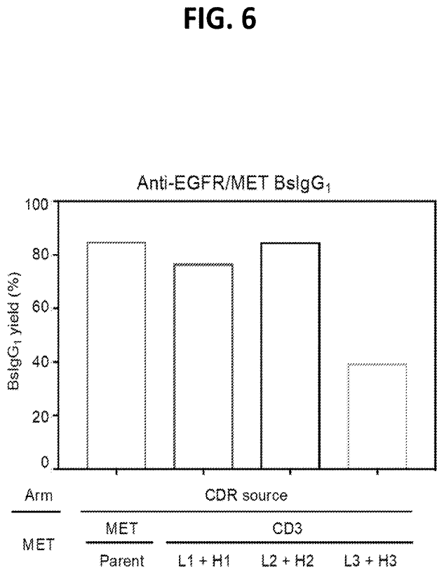

The sequence determinants in the anti-MET antibody that contribute to high bispecific yield of the anti-EGFR/MET BsIgG₁ were investigated. The amino acid sequence differences between the anti-EGFR and anti-MET antibodies are located entirely within the CDRs plus one additional framework region (FR) residue, V_{H} 94, immediately adjacent to CDR H3 (FIG. 4). The remaining FR, plus Cₖ and C_{H}1 constant domain sequences of these antibodies are identical (FIG. 4). CDR L3 and H3 are the CDRs that are most extensively involved at the V_{H}/V_{L} domain interface of the anti-MET antibody as evidenced by the X-ray crystallographic structure of the anti-MET Fab complexed with its antigen (Protein Data Bank (PDB) identification code 4K3J) (see Merchant et al. “Monovalent antibody design and mechanism of action of onartuzumab, a MET antagonist with anti-tumor activity as a therapeutic agent.” Proc Natl Acad Sci USA 2013; 110:E2987-96). These observations led to the hypothesis that CDR L3 and H3 of the anti-MET antibody may contribute to high bispecific yield for the anti-EGFR/MET BsIgG₁. Consistent with this idea, replacement of both CDR L3 and H3 of the anti-MET antibody with corresponding sequences from an anti-CD3 antibody led to substantial loss of bispecific yield (˜85% to 33%, FIG. 5A). In contrast, replacement of both CDR L3 and H3 of the anti-EGFR arm of the anti-EGFR/MET bispecific resulted in only a small reduction in BsIgG yield (˜85% to 75% FIG. 5A). Replacement of CDR L3 and H3 for both anti-EGFR and anti-MET arms resulted in random HC/LC pairing. These data support the notion that CDR L3 and H3 of anti-MET make major contributions to the high bispecific yield observed for the anti-EGFR/MET BsIgG₁, whereas CDR L3 and H3 of anti-EGFR make minor contributions. Replacement of CDR L1 and H1 or CDR L2 and H2 from the anti-MET antibody with corresponding anti-CD3 antibody sequences had little to no effect upon bispecific yield for the anti-EGFR/MET BsIgG (FIG. 6).

The Contributions of Residues Within the Anti-METCDR L3 and CDR H3 to the Yield of Anti-EGFR/MET BsIgG₁

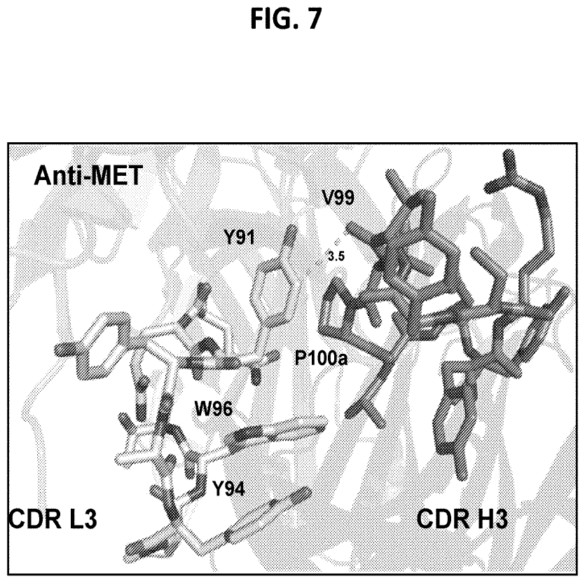

Next, the residues within CDRs L3 and H3 of anti-MET antibody that contribute to high bispecific yield of anti-EGFR/MET BsIgG₁ were investigated. The X-ray crystallographic structure of the anti-MET Fab (PDB accession code 4K3J) revealed contact residues between CDR L3 and H3 (FIG. 7) and was used to guide the selection of residues for mutational analysis. Alanine-scanning mutagenesis (Cunningham et al. “High-resolution epitope mapping of hGH-receptor interactions by alanine-scanning mutagenesis.” Science 1989; 244:1081-5) of anti-MET CDR L3 and H3 was used to map residues contributing to the high bispecific yield of anti-EGFR/MET BsIgG₁.

TABLE E1
Alanine Scanning Mutagenesis of CDR L3 and H3 Contact Residues for
an anti-MET antibody
Anti-EGFR/MET BsIgG₁

| Anti-MET variant | | |
|---|---|---|
| CDR L3 | CDR H3 | Yield (%) |
| Parent | Parent | 83.6 ± 3.5 |
| Y91A | Parent | 57.3 ± 1.0 |
| Y92A | Parent | 89.5 ± 0.2 |
| Y94A | Parent | 68.2 ± 4.9 |
| P95A | Parent | 85.8 ± 1.0 |
| W96A | Parent | 70.1 ± 0.9 |
| Y91A:Y94A | Parent | 22.6 ± 0.4 |
| Y91A:W96A | Parent | 35.1 ± 1.7 |
| Y94A:W96A | Parent | 56.0 ± 0.2 |
| Y91A:Y94A:W96A | Parent | 23.2 ± 0.2 |
| Parent | Y95A | 74.9 ± 0.9 |
| Parent | R96A | 78.3 ± 2.8 |
| Parent | S97A | 82.7 ± 3.9 |
| Parent | Y98A | 79.0 ± 0.1 |
| Parent | V99A | 79.8 ± 0.9 |
| Parent | T100A | 85.5 ± 0.7 |
| Parent | P100Aa | 64.7 ± 4.7 |
| Parent | V99A:P100aA | 72.8 ± 4.2 |

As shown in Table E1 above, the V_{L} Y91A mutation in CDR L3 gave the largest reduction in bispecific yield (84% to 57%) of any of the 12 single alanine mutants tested. As few as two alanine replacements in CDR L3, namely V_{L} Y91A : Y94A, abolished the high bispecific yield (84% to 23%). Thus, CDR L3 residues V_{L} Y91 and Y94 appear to make critical contributions to high bispecific yield for the anti-EGFR/MET BsIgG₁. The expression titers of all the mutants were comparable to the parent BsIgG₁ as estimated by the recovered yield from protein A chromatography (data not shown). The data shown in Table E1 represent the ±standard deviation for two independent experiments using optimized HC/LC DNA ratios (see Table B).

The affinities of the parental anti-MET Fab and a subset of the anti-MET Fab variants in Table E1 for MET were determined via surface plasmon resonance (SPR). The rates of association (kₒₙ), rates of dissociation (k_{off}) and binding affinities (K_{D}) are shown in Table E2 (n.d. indicates that binding was not detected). The P95A substitution in CDR L3 did not affect the binding of the anti-MET Fab variant to MET. Other single alanine substitutions in CDR L3 decreased affinity to varying degrees. Binding to antigen was not detected for anti-Met Fab variants having Y91A:Y94A or the Y91A:W96A double substitution in CDR L3.

TABLE E2
Parental anti-MET Fab and Fab variants

| | | kₒₙ | k_{off} | K_{D} |
|---|---|---|---|---|
| CDR L3 | CDR H3 | (×10⁴ M⁻¹s⁻¹) | (×10⁻⁴ s⁻¹) | (nM) |
| Parent | Parent | 17.9 | <0.1 | <0.05 |
| Y91A | Parent | 7.0 | 0.6 | 0.8 |
| Y92A | Parent | 17.2 | 1.9 | 1.1 |
| Y94A | Parent | 11.5 | 6.5 | 5.7 |
| P95A | Parent | 15.3 | <0.1 | <0.06 |
| W96A | Parent | 8.4 | 1.7 | 2.1 |
| Y91A:Y94A | Parent | n.d. | n.d. | n.d. |
| Y91A:W96A | Parent | n.d. | n.d. | n.d. |

The Contribution of the Anti-IL13CDR L3 and CDR H3 to the Yield of Anti-IL13/IL14 BsIgG₁

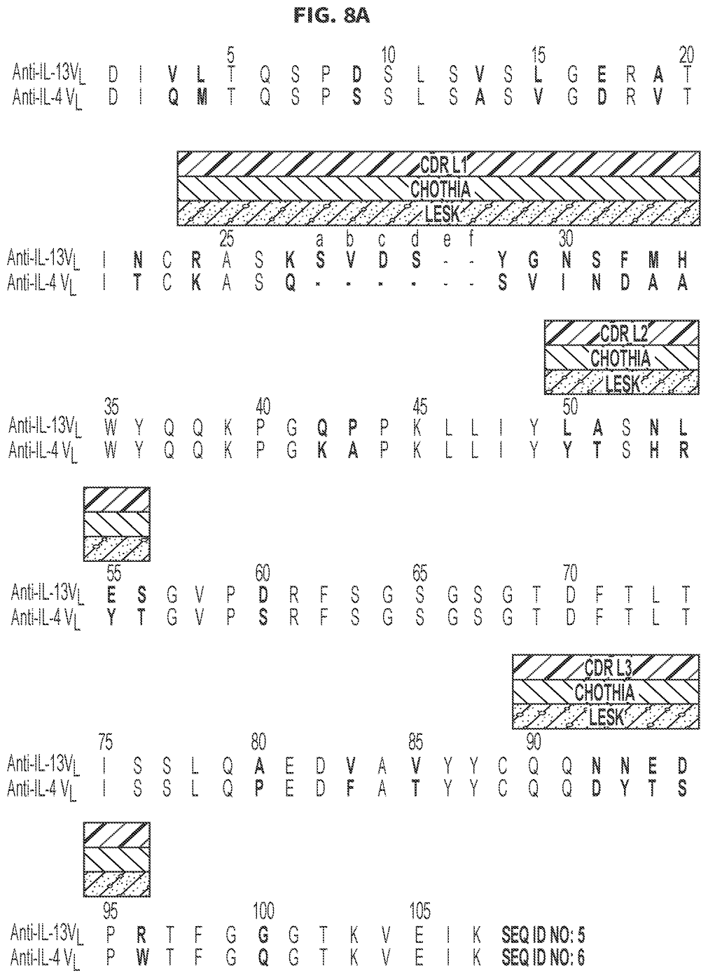

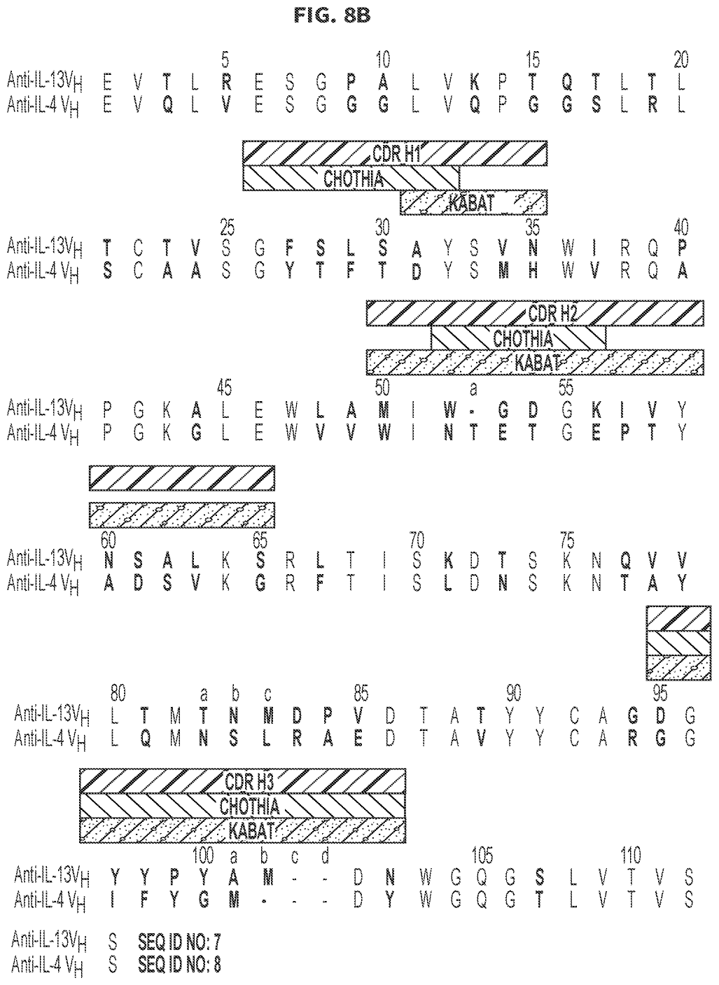

Given that specific residues in CDR L3 of the anti-MET antibody were found to be important for high bispecific yield for the anti-EGFR/MET BsIgG₁, it was postulated that similar principles may apply to the anti-IL-13 antibody in contributing to high bispecific yield of the anti-IL-13/IL-4 BsIgG₁. An analogous experimental strategy was used to investigate this possibility. One notable difference between these two antibody pairs is that the anti-IL-13 and anti-IL-4 antibodies differ in both their CDR and FR sequences (FIG. 8) whereas the anti-MET and anti-EGFR antibodies have identical FR sequences (except for V_{H} 94) and differ in their CDR sequences (FIG. 4).

Replacement of CDR L3 and H3 of the anti-IL-13 antibody with corresponding sequences from an anti-CD3 antibody led to substantial loss of bispecific yield of the anti-IL-13/IL-4 BsIgG₁ (˜72% to 37%, FIG. 5B). In contrast, a slight increase was observed when CDR L3 and H3 of the anti-IL-4 antibody were replaced in a similar manner (FIG. 5B). These results suggest that CDR L3 and H3 of the anti-IL-13 antibody contribute to high bispecific yield of the anti-IL-13/IL-4

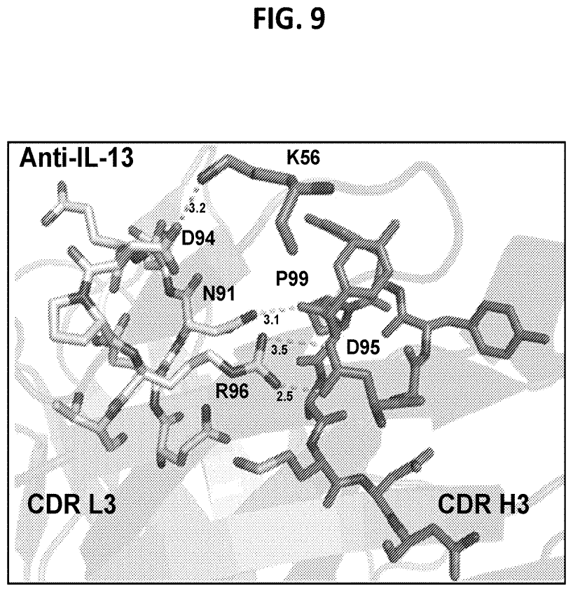

Alanine-scanning mutational analysis (Cunningham et al. infra) of anti-IL-13 CDR L3 and H3 was used to map residues contributing to the high bispecific yield of anti-IL-13/IL-4 BsIgG₁. The X-ray crystallographic structure of the anti-IL-13 Fab in complex with IL-13 (PDB accession code 4177, see Ultsch et al. “Structural basis of signaling blockade by anti-IL-13 antibody lebrikizumab.” J Mol Biol 2013; 425:1330-9) revealed the contact residues between CDR L3 and H3 (FIG. 9) and was used to select residues for mutational analysis (Table F1 below). The CDR L3 mutation V_{L} R96A gave the largest reduction in bispecific yield of any of the nine single alanine mutants tested for CDRs L3 and H3 and abolished the high bispecific yield (72% to 29%). As few as two alanine replacements in CDR H3, namely V_{H} D95A : P99A, also abolished the high bispecific yield (72% to 26%). The expression titers of all the mutants were comparable to the parent BsIgG₁ as estimated by the recovered yield from protein A chromatography (data not shown). The data shown in Table F1 represent the ±standard deviation for two independent experiments using optimized HC/LC DNA ratios (see Table B).

TABLE F1
Alanine Scanning Mutagenesis of CDR L3 and H3 Contact Residues for
an anti-IL13 antibody
Anti-IL-13/IL-4 BsIgG₁

| Anti-IL13 variant | | |
|---|---|---|
| CDR L3 | CDR H3 | Yield (%) |
| Parent | Parent | 71.8 ± 1.6 |
| N91A | Parent | 65.4 ± 2.1 |
| N92A | Parent | 69.7 ± 1.1 |
| D94A | Parent | 78.1 ± 3.3 |
| R96A | Parent | 28.7 ± 1.4 |
| N91A:D94A | Parent | 68.7 ± 3.5 |
| D94A:R96A | Parent | 24.8 ± 2.1 |
| N91A:D94A:R96A | Parent | 36.8 ± 0.1 |
| Parent | D95A | 55.9 ± 0.1 |
| Parent | Y97A | 77.0 ± 1.9 |
| Parent | Y98A | 63.7 ± 0.7 |
| Parent | P99A | 72.5 ± 1.3 |
| Parent | Y100A | 55.7 ± 2.8 |
| Parent | D95A:P99A | 26.1 ± 2.9 |

Thus, critical contributions to high bispecific yield can be made by CDR L3 and/or H3, as judged by both the anti-EGFR/MET and anti-IL-13/IL-4 BsIgG₁ studied here.

The affinities of the parental anti-IL-13 Fab and a subset of the anti-IL-13 Fab variants in Table F1 for IL-13 were determined via SPR. The rates of association (kₒₙ), rates of dissociation (k_{off}) and binding affinities (K_{D}) are shown in Table F2 (n.d. indicates that binding was not detected). Neither the N92A nor the D94A substitution in CDR L3 affected the binding of the anti-IL-13 Fab variant to IL-13. The R96A substitution in CDR L3 led to a ˜10-fold loss in binding affinity, as did the D94: R96A double substitution in CDR L3. Other single alanine substitutions in CDR H3 decreased affinity to varying degrees. Binding to antigen was not detected for the D95A:P99A double substitution in CDR H3.

Parental anti-IL-13 Fab and Fab variants

| | | kₒₙ | k_{off} | K_{D} |
|---|---|---|---|---|
| CDR L3 | CDR H3 | (×10⁴ M⁻¹s⁻¹) | (×10⁻⁴ s⁻¹) | (nM) |
| Parent | Parent | 117.1 | 0.5 | 0.05 |
| N92A | Parent | 103.0 | 0.3 | 0.03 |
| D94A | Parent | 124.3 | 0.3 | 0.02 |
| R96A | Parent | 82.5 | 4.4 | 0.5 |
| D94A:R96A | Parent | 52.8 | 3.7 | 0.7 |
| Parent | D95A | 88.2 | 11.1 | 1.3 |
| Parent | P99A | 150.4 | 26.9 | 1.8 |
| Parent | D95A:P99A | n.d. | n.d. | n.d. |

Effect of CDR L3 and CDR H3 on the Yield of BsIgG₁

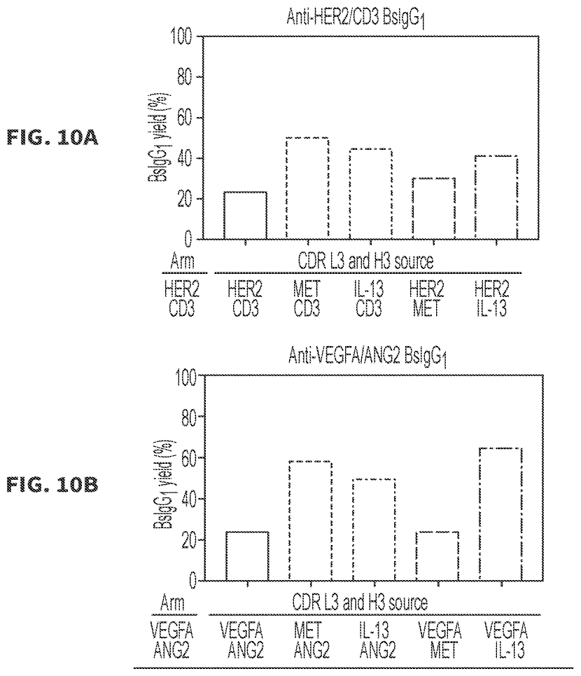

Next, a series of experiments was performed to determine whether CDR L3 and H3 from these antibodies could be sufficient for providing high bispecific yield for other antibody pairs. Two antibody pairs that have low bispecific yield, namely anti-HER2/CD3 (22-24%) and anti-VEGFA/ANG2 (24%) (see Table B and Dillon et al., infra) were selected, and the CDR L3 and H3 for one arm each of these two BsIgG₁ were replaced with corresponding CDR sequences from either the anti-MET or anti-IL-13 antibodies. A substantial increase in yield of BsIgGᵢ (from ˜24% up to 40-65%) was observed in three out of four CDR L3 and H3 recruitment cases for both anti-HER2/CD3 (FIG. 10A) and anti-VEGFA/ANG2 (FIG. 10B). The data presented in FIGS. 10A and 10B are from optimized LC DNA ratios. The data in FIGS. 10A and 10B indicate that recruitment of CDR L3 and H3 from antibodies with a cognate HC/LC pairing preference can enhance yield of BsIgG₁ with no pairing preference, but does not invariably do so.

The effect of the recruitment of a single critical residue from an anti-IL-13 antibody into other antibodies on BsIgG1 yield was investigated. See Table G1 below. Amino acid numbering is according to Kabat. The antibody containing the variable domain mutations is indicated in bold. Data shown is from optimized LC DNA ratios. Anti-VEGFC which has an aspartate residue at position 95 (D95) was not mutated.

TABLE G1
Recruitment of a Single Critical Residue from an anti-IL13 Antibody
into other Antibodies to Investigate Effect on BsIgG₁ Yield

| | | | BsIgG₁ yield |
|---|---|---|---|
| BsIgG₁ | CDR L3 | CDR H3 | (%) |
| Anti-HER2/CD3 | Parent | Parent | 24.0 |
| Anti-HER2/CD3 | T94D | Parent | 47.5 |
| Anti-HER2/CD3 | P96R | Parent | 40.1 |
| Anti-HER2/CD3 | Parent | W95D | 36.0 |
| Anti-VEGFA/ANG2 | Parent | Parent | 22.1 |
| Anti-VEGFA/ANG2 | V94D | Parent | 23.8 |
| Anti-VEGFA/ANG2 | W96R | Parent | 23.5 |
| Anti-VEGFA/ANG2 | Parent | Y95D | 22.7 |
| Anti-VEGFC/CD3 | Parent | Parent | 24.1 |
| Anti-VEGFC/CD3 | T94D | Parent (D95) | 44.0 |
| Anti-VEGFC/CD3 | P96R | Parent (D95) | 31.7 |

When two or more critical residues for pairing preference for anti-IL-13 were transplanted to the corresponding position in anti-HER2, anti-VEGFA or anti-VEGFC antibodies, some increase in bispecific yield was observed, albeit less than for the parental anti-IL-13/IL-4 BsIgG₁ (see Table G2 below). In Table G2, the antibody containing the variable domain mutations is indicated in bold, and the amino acid numbering is according to Kabat. The antibody containing the variable domain mutations is in bold underlined text. Data shown represent mean ±SD for two independent experiments using optimized LC DNA ratios. Anti-VEGFC, which has an aspartate residue at position 95 (D95), was not mutated.

TABLE G2
Recruitment of Critical Residues from an anti-IL13 Antibody
into other Antibodies to Investigate Effect on BsIgG₁ Yield

| | | | BsIgG₁ |
|---|---|---|---|
| | | | yield |
| BsIgG₁ | CDR L3 | CDR H3 | (%) |
| Anti-HER2/CD3 | Parent | Parent | 24.0 |
| Anti-HER2/CD3 | T94D:P96R | Parent | 31.8 |
| Anti-HER2/CD3 | Parent | W95D | 36.0 |
| Anti-HER2/CD3 | T94D:P96R | W95D | 47.4 |
| Anti-VEGFA/ANG2 | Parent | Parent | 22.1 |
| Anti-VEGFA/ANG2 | V94D:W96R | Parent | 52.5 |
| Anti-VEGFA/ANG2 | Parent | Y95D | 22.7 |
| Anti-VEGFA/ANG2 | V94D:W96R | Y95D | 59.1 |
| Anti-VEGFC/CD3 | Parent | Parent (D95) | 24.1 |
| Anti-VEGFC/CD3 | T94D:P96R | Parent (D95) | 50.4 |

Together, these results suggested that charged residues (such as D and R) at positions 94 and 96 of CDR L3 (Kabat numbering) and at position 95 of CDR H3 (Kabat numbering) can impart pairing preference for some but not all antibody pairs.

The affinities of the parental anti-HER2, anti-VEGFA, and anti-VEGFC Fabs and a subset of the anti-HER2, anti-VEGFA, and anti-VEGFC Fab variants in Tables G1 and G2 for their respective targets were determined via SPR. The rates of association (kₒₙ), rates of dissociation (k_{off}) and binding affinities (K_{D}) are shown in Table G3 (n.d. indicates that binding was not detected). Transferring critical residues from anti-IL13 to other antibodies led to loss of binding affinity. Notably, the T94D substitution in the CDR-L3 of anti-HER2 increased the BsIgG₁ yield of the anti-HER²/anti-CD3 BsAb from 24% to almost 50%, yet only decreased the affinity of anti-HER2 for HER2 by 20-fold. Similarly, the V94D:W96R double substitution in the CDR-L3 of VEGFA increased the BsIgG₁ yield of the anti-VEGFA/anti-ANG2 BsAb from about 22% to about 52%, yet only decreased the affinity of anti-VEGFA for VEGFA by about 20 fold.

TABLE G3

| | | | kₒₙ | k_{off} | K_{D} |
|---|---|---|---|---|---|
| Fab | CDR L3 | CDR H3 | (×10⁴ M⁻¹s⁻¹) | (×10⁻⁴ s¹) | (nM) |
| Anti- | Parent | Parent | 10.4 | 1.3 | 1.2 |
| HER2 | T94D | Parent | 6.9 | 16.8 | 24.4 |
| | P96R | Parent | 7.0 | 149.5 | 212.9 |
| | Parent | W95D | 8.0 | 29.4 | 36.5 |
| | T94D:P96R | Parent | n.d. | n.d. | n.d. |
| | T94D:P96R | W95D | n.d. | n.d. | n.d. |
| Anti- | Parent | Parent | 65.4 | <0.1 | <0.015 |
| VEGFA | V94D | Parent | 59.8 | <0.1 | <0.016 |
| | W96R | Parent | 13.3 | 9.1 | 6.8 |
| | Parent | Y95D | 92.6 | 6.0 | 0.6 |
| | V94D:W96R | Parent | 163.8 | 4.7 | 0.3 |
| | T94D:P96R | W95D | n.d. | n.d. | n.d. |
| Anti- | Parent | Parent | 17.1 | 14.1 | 8.2 |
| VEGFC | V94D | Parent | n.d. | n.d. | n.d. |
| | W96R | Parent | n.d. | n.d. | n.d. |
| | Parent | Y95D | n.d. | n.d. | n.d. |

In contrast to the results shown in Tables G1 and G2, when critical residues for pairing preference for anti-cMet were transplanted to the corresponding position in anti-HER2, anti-VEGFA or anti-VEGFC antibodies, little increase in bispecific yield was observed in most cases. See Table G4 below. In Table G4, the antibody containing the variable domain mutations is indicated in bold, and the amino acid numbering is according to Kabat.

TABLE G4
Recruitment of Critical Residues from an anti-cMet Antibody
into other Antibodies to Investigate Effect on BsIgG₁ Yield

| | | | BsIgG₁ yield |
|---|---|---|---|
| BsIgG₁ | CDR L3 | CDR H3 | (%) |
| Anti-HER2/CD3 | Parent | Parent | 24.0 |
| Anti-HER2/CD3 | H91Y | Parent | 23.6 |
| Anti-HER2/CD3 | T94Y | Parent | 31.0 |
| Anti-HER2/CD3 | P96W | Parent | 26.2 |
| Anti-HER2/CD3 | H91Y:T94Y | Parent | 24.2 |
| Anti-HER2/CD3 | H91Y:P96W | Parent | 23.4 |
| Anti-HER2/CD3 | T94Y:P96W | Parent | 22.7 |
| Anti-HER2/CD3 | H91Y:T94Y:P96W | Parent | 23.6 |
| Anti-VEGFA/ANG2 | Parent (Y91, W96) | Parent | 22.1 |
| Anti-VEGFA/ANG2 | (Y91)V94Y(W96) | Parent | 23.6 |
| Anti-VEGFC/CD3 | Parent | Parent | 23.9 |
| Anti-VEGFC/CD3 | S91Y | Parent | 22.6 |
| Anti-VEGFC/CD3 | T94Y | Parent | 33.6 |
| Anti-VEGFC/CD3 | P96W | Parent | 47.7 |
| Anti-VEGFC/CD3 | S91Y:T94Y | Parent | 22.4 |
| Anti-VEGFC/CD3 | S91Y:P96W | Parent | 59.0 |
| Anti-VEGFC/CD3 | T94Y: P96W | Parent | 36.4 |
| Anti-VEGFC/CD3 | S91Y:T94Y:P96W | Parent | 47.8 |
| Anti-HER2/EGFR | Parent | Parent | 21.4 |
| Anti-HER2/EGFR | H91Y:T94Y | Parent | 22.3 |
| Anti-HER2/EGFR | H91Y:P96W | Parent | 24.2 |
| Anti-HER2/EGFR | T94Y:P96W | Parent | 23.4 |
| Anti-HER2/EGFR | H91Y:T94Y:P96W | Parent | 33.6 |

The Contribution of Interchain Disulfide Bonds on Yield of BsIgG₁

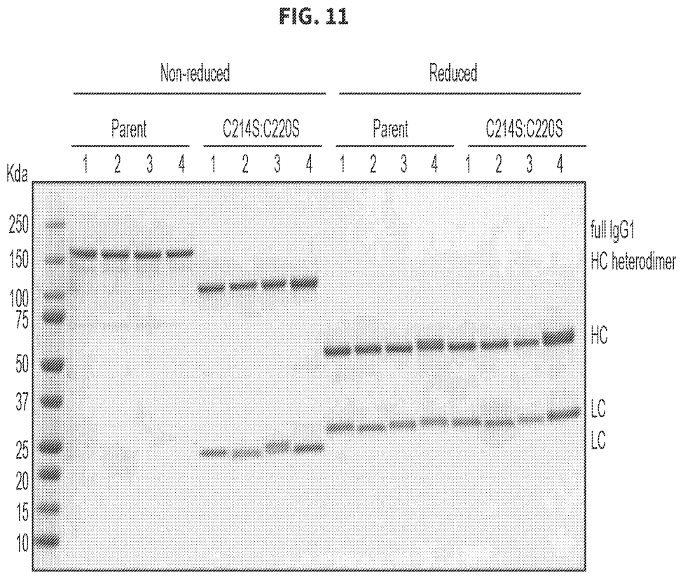

Previously, it was hypothesized that formation of the interchain disulfide bond between the HC and LC acts as a kinetic trap that prevents chain exchange (Dillon et al., infra). Experiments were performed to investigate whether the disulfide bond between HC and LC affects the bispecific yield for two BsIgG₁ with a pronounced cognate chain preference (anti-EGFR/MET and anti-IL-13/IL-4) and two controls with random HC/LC pairing (anti-HER2/CD3 and anti-VEGFA/VEGFC). Briefly, BsIgG₁ variants lacking the inter-chain disulfide bond were generated using cysteine to serine mutations: LC C214S and HC C220S. Removal of the inter-chain disulfide bond in the engineered variants was verified by SDS PAGE. Samples were electrophoresed under either reducing or non-reducing conditions, as indicated in FIG. 11. Four different BsIgG1 were analyzed: anti-HER2/CD3 (lanes 1); anti-VEGFA/VEGFC (lanes 2); anti-EGFR/MET (lanes 3); and anti-IL13/IL14 (lanes 4). As shown in Table H below, no clear evidence was found that the inter-chain disulfide bond affects BsIgG₁ yield for any of the four antibody pairs tested as judged by native mass spectrometry. The yield of BsIgG₁ of the parental and the disulfide bond engineered variants were similar. The data in Table H are the mean ±standard deviations for three biological replicates using optimized DNA light chain ratios.

TABLE H
Mutational Analysis to Determine the Effect of the Disulfide
Bond between HC and LC on BsIgG₁ yield.

| | BsIgG₁ yield (%) | |
|---|---|---|
| | Parent with HC/LC | Variant without HC/LC |
| BsIgG₁ | disulfide bond | disulfide bond |
| Anti-EGFR/MET | 81.1 ± 1.4 | 82.8 ± 2.6 |
| Anti-IL-13/IL-4 | 73.3 ± 4.5 | 75.1 ± 0.8 |
| Anti-HER2/CD3 | 24.5 ± 0.8 | 27.0 ± 2.4 |
| Anti-VEGFA/VEGFC | 28.8 ± 5.9 | 38.0 ± 6.0 |

In summary, this study demonstrates that a cognate HC/LC pairing preference in producing BsIgG in single cells is a common phenomenon that is highly dependent upon the specific antibody pair. Mechanistically, this chain pairing preference can be strongly influenced by residues in CDR H3 and L3. Practically, this pairing preference can be utilized to reduce the number of Fab mutations used to drive the production of BsIgG₁ and potentially BsIgG of other isotypes in single cells.

### Additional References

Brinkmann U, Kontermann R E. The making of bispecific antibodies. mAbs 2017; 9:182-212.

Carter P J, Lazar G A. Next generation antibody drugs: pursuit of the ‘high-hanging fruit’. Nat Rev Drug Discov 2018; 17:197-223.

Sanford M. Blinatumomab: first global approval. Drugs 2015; 75:321-7.

Oldenburg J, Mahlangu J N, Kim B, Schmitt C, Callaghan M U, Young G, Santagostino E, Kruse-Jarres R, Negrier C, Kessler C, et al. Emicizumab prophylaxis in hemophilia A with inhibitors. N Engl J Med 2017; 377:809-18.

Scott L J, Kim E S. Emicizumab-kxwh: First Global Approval. Drugs 2018; 78:269-74.

Suresh M R, Cuello A C, Milstein C. Bispecific monoclonal antibodies from hybrid hybridomas. Methods Enzymol 1986; 121:210-28.

Fischer N, Elson G, Magistrelli G, Dheilly E, Fouque N, Laurendon A, Gueneau F, Ravn U, Depoisier J F, Moine V, et al. Exploiting light chains for the scalable generation and platform purification of native human bispecific IgG. Nat Commun 2015; 6:6113.

Strop P, Ho W H, Boustany L M, Abdiche Y N, Lindquist K C, Farias S E, Rickert M, Appah C T, Pascua E, Radcliffe T, et al. Generating bispecific human IgG1 and IgG2 antibodies from any antibody pair. J Mol Biol 2012; 420:204-19.

Vaks L, Litvak-Greenfeld D, Dror S, Matatov G, Nahary L, Shapira S, Hakim R, Alroy I, Benhar I. Design principles for bispecific IgGs, opportunities and pitfalls of artificial disulfide bonds. Antibodies 2018; 7.

Schaefer W, Volger H R, Lorenz S, Imhof-Jung S, Regula J T, Klein C, Molhoj M. Heavy and light chain pairing of bivalent quadroma and knobs-into-holes antibodies analyzed by UHR-ESI-QTOF mass spectrometry. MAbs 2016; 8:49-55.

Bönisch M, Sellmann C, Maresch D, Halbig C, Becker S, Toleikis L, Hock B, Ruker F. Novel C_{H}1:CL interfaces that enhance correct light chain pairing in heterodimeric bispecific antibodies. Protein Eng Des Sel 2017; 30:685-96.

Kitazawa T, Igawa T, Sampei Z, Muto A, Kojima T, Soeda T, Yoshihashi K, Okuyama-Nishida Y, Saito H, Tsunoda H, et al. A bispecific antibody to factors IXa and X restores factor VIII hemostatic activity in a hemophilia A model. Nature Medicine 2012; 18:1570-4.

Sampei Z, Igawa T, Soeda T, Funaki M, Yoshihashi K, Kitazawa T, Muto A, Kojima T, Nakamura S, Hattori K. Non-antigen-contacting region of an asymmetric bispecific antibody to factors IXa/X significantly affects factor VIII-mimetic activity. MAbs 2015; 7:120-8.

Sampei Z, Igawa T, Soeda T, Okuyama-Nishida Y, Moriyama C, Wakabayashi T, Tanaka E, Muto A, Kojima T, Kitazawa T, et al. Identification and multidimensional optimization of an asymmetric bispecific IgG antibody mimicking the function of factor VIII cofactor activity. PLoS One 2013; 8:e57479.

Carter P J. Introduction to current and future protein therapeutics: a protein engineering perspective. Exp Cell Res 2011.

Wu H, Pfarr D S, Johnson S, Brewah Y A, Woods R M, Patel N K, White W I, Young J F, Kiener P A. Development of motavizumab, an ultra-potent antibody for the prevention of respiratory syncytial virus infection in the upper and lower respiratory tract. J Mol Biol 2007; 368:652-65.

Cooke H A, Arndt J, Quan C, Shapiro R I, Wen D, Foley S, Vecchi M M, Preyer M. EFab domain substitution as a solution to the light-chain pairing problem of bispecific antibodies. MAbs 2018; 10:1248-59.

Tiller K E, Li L, Kumar S, Julian M C, Garde S, Tessier P M. Arginine mutations in antibody complementarity-determining regions display context-dependent affinity/specificity trade-offs. J Biol Chem 2017; 292:16638-52.

Dashivets T, Stracke J, Dengl S, Knaupp A, Pollmann J, Buchner J, Schlothauer T. Oxidation in the complementarity-determining regions differentially influences the properties of therapeutic antibodies. MAbs 2016; 8:1525-35.

Lamberth K, Reedtz-Runge S L, Simon J, Klementyeva K, Pandey G S, Padkjaer S B, Pascal V, Leon I R, Gudme C N, Buus S, et al. Post hoc assessment of the immunogenicity of bioengineered factor VIIa demonstrates the use of preclinical tools. Sci Transl Med 2017; 9.

Harding F A, Stickler M M, Razo J, DuBridge R. The immunogenicity of humanized and fully human antibodies. mAbs 2014; 2:256-65.

Sekiguchi N, Kubo C, Takahashi A, Muraoka K, Takeiri A, Ito S, Yano M, Mimoto F, Maeda A, Iwayanagi Y, et al. MHC-associated peptide proteomics enabling highly sensitive detection of immunogenic sequences for the development of therapeutic antibodies with low immunogenicity. MAbs 2018; 10:1168-81.

Schachner L, Han G, Dillon M, Zhou J, McCarty L, Ellerman D, Yin Y, Spiess C, Lill JR, Carter P J, et al. Characterization of chain pairing variants of bispecific IgG expressed in a single host cell by high-resolution native and denaturing mass spectrometry. Anal Chem 2016; 88:12122-7.

### Example 3

### Affinity Maturation of Modified Antibodies Generated in Example 2

The exemplary antibodies in Table I, which were generated in Example 2, are subject to affinity maturation to improve their affinities for their respective target antigens.

TABLE I

| | | | Exemplary Candidates |
|---|---|---|---|
| | | | for Affinity Maturation |
| | | | (by ~20-40 fold to restore |
| Antibody | CDR L3* | CDR H3* | parental affinity) |
| Anti-HER2 | T94D | Parent | K_{D} of modified antibody is |
| | | | ~20x lower than that of |
| | | | unmodified parent** |
| | Parent | W95D | K_{D} of modified antibody is |
| | | | ~30x lower than that of |
| | | | unmodified parent** |
| Anti-VEGFA | V94D | Parent | K_{D} of modified antibody |
| | | | is comparable to that of |
| | | | unmodified parent (and |
| | | | optionally can be further |
| | | | affinity matured, if |
| | | | desired)** |
| | Parent | Y95D | K_{D} of modified antibody is |
| | | | ~38x lower than that of |
| | | | unmodified parent** |
| | V94D:W96R | Parent | K_{D} of modified antibody is |
| | | | ~20x lower than that of |
| | | | unmodified parent** |

*The amino acid numbering is according to Kabat.
**See Table G3.

Briefly, mutations are introduced into the CDRs of the antibodies in Table Ito generate one or more polypeptide libraries (e.g., phage display or cell surface display libraries) for each antibody. The amino acid substitution(s) that were introduced into the CDR-L3 and/or CDR-H3 of each antibody to improve bispecific yield (see Table I) remain fixed and are not randomized during library construction. Each library is then screened by panning or cell sorting, e.g., as described in Wark et al. (2006) Adv Drug Deliv Rev. 58: 657-670; Rajpal et al. (2005) Proc Natl Acad Sci USA. 102: 8466-8471, to identify antibody variants that bind target antigen (i.e., HER2, VEGFA, or VEGFC) with high affinity. Such variants are then isolated, and their affinities for their target antigen are determined, e.g., via surface plasmon resonance, and compared to the affinities of the antibodies shown in Table I and to the parental antibodies from which the antibodies in Table I were derived (see, e.g. Table G3). At least one round (such as at least any one of 2, 3, 4, 5, 6, 7, 8, 9, or 10 rounds) of affinity maturation is performed to identify high-affinty anti-HER2 variants, high-affinty anti-VEGFA variants, and high-affinty anti-VEGFC variants. The sequences of the antibody variants with high affinities for their respective target antigen are determined.

Next, the variants identified in the screens described above are analyzed further to assess their effects on bispecific antibody yield. Briefly, high-affinity anti-HER2, anti-VEGFA, and anti-VEGFC variants are reformatted as bispecific antibodies. Exemplary bispecific antibodies include, but are not limited to, e.g., anti-HER2/anti-CD3, anti-VEGFA/anti-ANG2, and anti-VEGFC/anti-CD3 (see Tables G1 and G2 above). The bispecific antibodies are expressed and purified, e.g., according to methods detailed in Example 1. The yield of correctly assembled bispecific antibody is assessed, e.g., via size exclusion chromatography, high resolution LCMS, and/or SDS-PAGE gel analysis, as detailed in Example 1. Control experiments using, e.g., bispecific antibodies shown in Tables G1 and G2, are performed in parallel The yield of bispecific antibodies comprising a high-affinity anti-HER2 antibody variant, a high-affinity anti-VEGFA variant, or an anti-VEGFC variant identified via library screen is compared to the yield of bispecific antibodies comprising an anti-HER2, an anti-VEGFA, or an anti-VEGFC antibody shown in Table I Additional modified antibodies that are subject to one or more affinity maturation steps and assayed further for improved affinity and BsAb yield, i.e., as described above, are shown in Table G3.

### Additional References

Merchant et al. (2013) Proc Natl Acad Sci USA. 110(32): E2987-96

Julian et al. (2017) Scientific Reports. 7: 45259

Tiller et al. (2017) Front. Immunol. 8: 986

Koenig et al. (2017) Proc Natl Acad Sci USA. 114(4): E486-E495

Yamashita et al. (2019) Structure. 27, 519-527

Payandeh et al. (2019) J Cell Biochem. 120: 940-950

Richter et al. (2019) mAbs. 11(1): 166-177

Cisneros et al. (2019) Mol. Syst. Des. Eng. 4: 737-746

The preceding Examples are offered for illustrative purposes only, and are not intended to limit the scope of the present invention in any way. Various modifications of the invention in addition to those shown and described herein will become apparent to those skilled in the art from the foregoing description and fall within the scope of the appended claims.

1. A method of improving preferential pairing of a heavy chain and a light chain of an antibody, comprising the step of substituting at least one amino acid at position 94 of a light chain variable domain (V_{L}) or position 96 of the V_{L}, from a non-charged residue to a charged residue selected from the group consisting of aspartic acid (D), arginine (R), glutamic acid (E), and lysine (K), wherein the amino acid numbering is according to Kabat.

2. The method of claim 1, comprising the step of substituting each of the amino acids at position 94 and position 96 from a non-charged residue to a charged residue.

3. The method of claim 1 or 2, wherein the amino acid at position 94 is substituted with D.

4. The method of any one of claims 1-3, wherein the amino acid at position 96 is substituted with R.

5. The method of any one of claims 1-4, wherein the amino acid at position 94 is substituted with D and the amino acid at position 96 is substituted with R.

6. The method of any one of claims 1-5, wherein the amino acid at position 95 of a heavy chain variable domain (V_{H}) is substituted from a non-charged residue to a charged residue selected from the group consisting of aspartic acid (D), arginine (R), glutamic acid (E), and lysine (K), wherein the amino acid numbering is according to Kabat.

7. The method of any one of claims 1-6, wherein the amino acid at position 94 of the V_{L} is substituted with D, the amino acid at position 96 of the V_{L} is substituted with R, and the amino acid at position 95 of the V_{H} is substituted with D.

8. The method of any one of claims 1-7, further comprising subjecting the antibody to at least one affinity maturation step, wherein the substituted amino acid at position 94 of the V_{L} is not randomized.

9. The method of claim 8, wherein the substituted amino acid at position 96 of the V_{L} is not randomized.

10. The method of claim 8 or 9, wherein the substituted amino acid at position 95 of the V_{H} is not randomized.

11. The method of any one of claims 1-10, wherein the antibody is an antibody fragment selected from the group consisting of: a Fab, a Fab′, an F(ab′)₂, a one-armed antibody, and scFv, or an Fv.

12. The method of claim any one of claims 1-11, wherein the antibody is a human, humanized, or chimeric antibody.

13. The method of any one of claims 1-12, wherein the antibody comprises a human IgG Fc region.

14. The method of claim 13, wherein the human IgG Fc region is a human IgG1, human IgG2, human IgG3, or human IgG4 Fc region.

15. The method of any one of claims 1-14, wherein the antibody is a monospecific antibody.

16. The method of any one of claims 1-14, wherein the antibody is a multispecific antibody.

17. The method of claim 16, wherein the multispecific antibody is a bispecific antibody.

18. The method of claim 14 wherein the bispecific antibody comprises a first C_{H}2 domain (C_{H}2₁), a first C_{H}3 domain (C_{H}3₁), a second C_{H}2 domain (C_{H}2₂), and a second C_{H}3 domain;
wherein C_{H}3₂ is altered so that within the C_{H}3₁/C_{H}3₂ interface, one or more amino acid residues are replaced with one or more amino acid residues having a larger side chain volume, thereby generating a protuberance on the surface of C_{H}3₂ that interacts with C_{H}3₁; and
wherein C_{H}3₁ is altered so that within the C_{H}3₁/C_{H}3₂ interface, one or more amino acid residues are replaced amino acid residues having a smaller side chain volume, thereby generating a cavity on the surface of C_{H}3₁ that interacts with C_{H}3₂.

19. The method of claim 14, wherein the bispecific antibody comprises a first C_{H}2 domain (C_{H}2₁), a first C_{H}3 domain (C_{H}3₁), a second C_{H}2 domain (C_{H}2₂), and a second C_{H}3 domain;
wherein C_{H}3₁ is altered so that within the C_{H}3₁/C_{H}3₂ interface, one or more amino acid residues are replaced with one or more amino acid residues having a larger side chain volume, thereby generating a protuberance on the surface of C_{H}3₁ that interacts with C_{H}3₂; and
wherein C_{H}3₂ is altered so that within the C_{H}3₁/C_{H}3₂ interface, one or more amino acid residues are replaced amino acid residues having a smaller side chain volume, thereby generating a cavity on the surface of C_{H}3₂ that interacts with C_{H}3₁.

20. The method of claim 15 or 16, wherein the protuberance is a knob mutation.

21. The method of claim 17, wherein the knob mutation comprises T366W, wherein amino acid numbering is according to the EU index.

22. The method of any one of claims 15-18, wherein the cavity is a hole mutation.

23. The method of claim 22, wherein the hole mutation comprises at least one, at least two, or all three of T366S, L368A, and Y407V, wherein amino acid numbering is according to the EU index.

24. An antibody produced by the method of any one of claims 1-23.


SEQUENCE LISTING (8 sequences)

SEQ ID NO: 1 - protein, 113 aa, Artificial Sequence [sequence omitted]

SEQ ID NO: 2 - protein, 107 aa, Artificial Sequence
  Synthetic Construct
DIQMTQSPSSLSASVGDRVTITCRASQDVSTAVAWYQQKPGKAPKLLIYSASFLYSGVPSRFSGSGSGTDFTLTISSLQPEDFATYYCQQSYPTPYTFGQGTKVEIK

SEQ ID NO: 3 - protein, 119 aa, Artificial Sequence [sequence omitted]

SEQ ID NO: 4 - protein, 121 aa, Artificial Sequence
  Synthetic Construct
EVQLVESGGGLVQPGGSLRLSCAASGFTFTGNWIHWVRQAPGKGLEWVGEISPSGGYTDYADSVKGRFTISADTSKNTAYLQMNSLRAEDTAVYYCARESRVSYEAAMDYWGQGTLVTVSS

SEQ ID NO: 5 - protein, 111 aa, Artificial Sequence
  Synthetic Construct
DIVLTQSPDSLSVSLGERATINCRASKSVDSYGNSFMHWYQQKPGQPPKLLIYLASNLESGVPDRFSGSGSGTDFTLTISSLQAEDVAVYYCQQNNEDPRTFGGGTKVEIK

SEQ ID NO: 6 - protein, 107 aa, Artificial Sequence
  Synthetic Construct
DIQMTQSPSSLSASVGDRVTITCKASQSVINDAAWYQQKPGKAPKLLIYYTSHRYTGVPSRFSGSGSGTDFTLTISSLQPEDFATYYCQQDYTSPWTFGQGTKVEIK

SEQ ID NO: 7 - protein, 118 aa, Artificial Sequence
  Synthetic Construct
EVTLRESGPALVKPTQTLTLTCTVSGFSLSAYSVNWIRQPPGKALEWLAMIWGDGKIVYNSALKSRLTISKDTSKNQVVLTMTNMDPVDTATYYCAGDGYYPYAMDNWGQGSLVTVSS

SEQ ID NO: 8 - protein, 118 aa, Artificial Sequence
  Synthetic Construct
EVQLVESGGGLVQPGGSLRLSCAASGYTFTDYSMHWVRQAPGKGLEWVVWINTETGEPTYADSVKGRFTISLDNSKNTAYLQMNSLRAEDTAVYYCARGGIFYGMDYWGQGTLVTVSS